(19) United States
(12) Patent Application Publication
(10) Pub. No.: US 2016/0318887 A1
(43) Pub. Date: Nov. 3, 2016

(54) AMIDOPHENOXYPROPANOLAMINES

(71) Applicant: MEDIZINISCHE UNIVERSITÄT WIEN, Wien (AT)

(72) Inventors: Hubert Gstach, Wien (AT); Peter Chiba, Wien (AT); Matthias Mastalir, Wien (AT)

(21) Appl. No.: 15/204,585

(22) Filed: Jul. 7, 2016

Related U.S. Application Data
(63) Continuation of application No. 14/403,916, filed on Nov. 25, 2014, now abandoned.

(30) Foreign Application Priority Data
Jun. 13, 2012 (EP) 12171729.2

(51) Int. Cl. C07D 295/088; C07C 235/66; C07D 229/02; C07D 211/14; C07C 235/60; C07C 235/64

(52) U.S. Cl. CPC C07D 295/088; C07C 235/60; C07C 235/64; C07D 229/02; C07D 211/14; C07C 235/66; C07C 2103/74; C07C 2101/02; C07C 2101/20; C07C 2101/18

(57) ABSTRACT

The use of compounds of formula

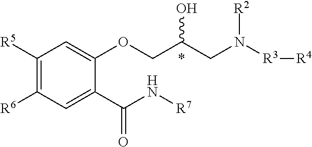

wherein R², R³, R⁴, R⁵, R⁶ and R⁷ have several meanings, for the treatment of disorders mediated by protozoan organisms, novel compounds of the above formula and intermediates for the preparation of such compounds, pharmaceutical compositions comprising such novel compounds, a method of treating disorders mediated by protozoan organisms comprising administering such compounds, optionally together with a second drug substance, to a subject in need thereof and the use of such compounds, whenever comprising a photoaffinity label, for the identification of the molecular target(s) of arylamino alcohol antimalarials.

### CROSS REFERENCE TO RELATED APPLICATIONS

This application is a continuation of U.S. patent application Ser. No. 14/403,916, filed Nov. 25, 2014, which is a 371 application of International Patent Application No. PCT/EP2013/061880, filed Jun. 10, 2013, which claims the benefit of European Patent Application No. EP 12171729.2, filed Jun. 13, 2012, the disclosures of which are incorporated herein in their entirety.

### BACKGROUND

The present invention relates to amidophenoxypropanolamines which were found to be active in the treatment of infections mediated, e.g. caused, by protozoan organisms, resulting in diseases such as malaria, Chagas disease, sleeping sickness, trichomoniasis, leishmaniasis, giardiasis, amebiasis, toxoplasmosis, trypanosomiasis (animal sleeping sickness), babesiosis, theileriosis, coccidiosis.

Numerous diseases are mediated, e.g. caused, by infections with protozoan organisms, such as apicomplexa, kinetoplastids, microsporidia, plasmodia. The latter are responsible for malaria the most prevalent disease in South Asia and sub-Saharan Africa.

Malaria is caused by four protozoan Plasmodium parasites that invade and destroy erythrocytes in affected individuals. P. falciparum is the most prevalent and deadly representative of the genus, particularly in sub-Saharan Africa. Still close to one million people, mostly children and pregnant women in developing countries, die of malaria each year although malaria represents a treatable and preventable disease (1).

WHO has put forward an agenda for the development of novel medicines with the ultimate goal of global eradication of the disease. In addition, treatment-guidelines have been issued, which emphasize the importance of artemisinin based combination therapies (ACT). The emergence of drug-resistance has been observed for all classes of drugs, including artemisinin derivatives (2). The latter has to be taken seriously, because a loss of activity of artemisinin drugs in ACTs would lead to rapid development of resistance against the partner drugs. Recent investigations have added proof to earlier reports, that P. falciparum is becoming resistant to the front line treatment of malaria. Resistant strains have started spreading in Southeast Asia recently (3-4). A spillover of artemisinin resistant P. falciparum into sub-Saharan Africa would cause a health disaster.

The most prescribed ACT for treatment of malaria is Coartem® with Lumefantrine® and Artemether® as drug components. Coartem® is included in the WHO Model List of Essential Medicines since 2002. Lumefantrine conforms to the arylamino alcohol group of antimalarials that includes quinine and mefloquine:

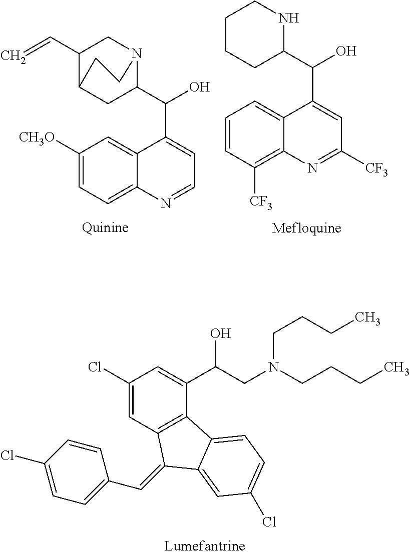

The latter two drugs were extensively used as monotherapies, which resulted in rapid decrease of efficacy in South East Asia (5). Lumefantrine has never been used as a monotherapy and development of resistance against the drug is retarded by combination with the rapidly acting artemisinin partner drug Artemether®. But in the light of spreading artemisinin resistance the most essential pillar in ACT of P. falciparum infections is endangered to be faced with development of resistance as seen for other compounds from the group of arylamino alcohols.

There is a serious risk for loss of the arylamino alcohol group of antimalarials on the whole. The current number of novel antimalarial drugs in the pipeline for late stage development is extremely low and dominated by combinations of old drugs. Additionally, one of the limiting factors in antimalarial drug research is the lack of understanding the mode of action of most drugs. Without knowledge about the molecular targets a rational approach in finding new drugs is hampered.

Because of the undisputed clinical usefulness of arylamino alcohol antimalarials in artemisinin combination regimens it could be desirable to expand this compound class by alternative novel scaffolds. Such a scaffold is represented by propafenone which is a marketed class 1c antiarrhythmicum. Propafenone (compound of formula A below) has been shown to have good antimalarial activity (6-8). The compound belongs chemically to the arylamino alcohol group and served as a promising starting point for extension of this group of antimalarials with different, potentially better pharmacological properties compared to quinine, mefloquine or lumefantrine, respectively. The antiarrhythmic effect of propafenone was tried to be engineered out by proper chemical modifications. Introduction of modified amino substituents into the propafenone scaffold achieved this goal (8). The propafenone scaffold has been extensively derivatized, including substitution of the methylene group in α-position to the ketone functionality by a nitrogen atom. The one atom modification transferred the scaffold of propafenone of formula A to the class of salicylamides of formula B as shown below and reduced the antiarrhythmic potency to a level which was no longer useful in therapy of cardiac disorders (9).

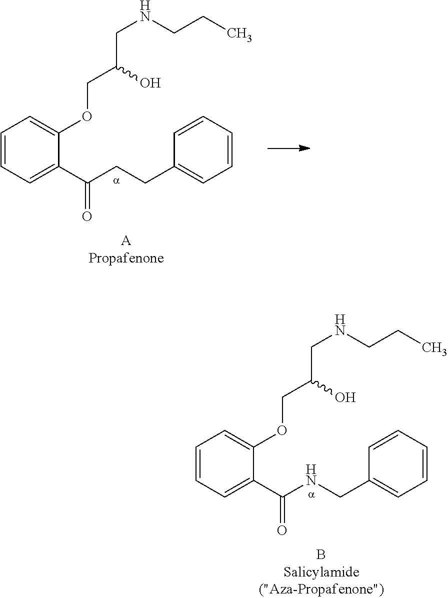 [SMILES: CCCNCC(O)COc1ccccc1C(=O)CCc1ccccc1 ; CCCNCC(O)COc1ccccc1C(=O)NCc1ccccc1]

This observation adds an alternative strategy for out-engineering the antiarrhythmic activity of the propafenone scaffold other than modification of the amine residue. But transformation of propafenone (A) to “aza-propafenone” (B) is deleterious to antimalarial activity. The one atom modification led to a seventy-fold decrease in IC₅₀-value for chloroquine (CQ) sensitive (3D7) strain of P falciparum compared to propafenone (A) (data not shown).

### SUMMARY

Now, surprisingly novel amidophenoxypropanolamines were found which show exceptional high activity against chloroquine (CQ) sensitive 3D7 (NF54) strain of P. falciparum and against CQ resistant K1 strain. Selected novel compounds of the present invention show in vivo antimalarial efficacy in a P. berghei rodent malaria model.

### DETAILED DESCRIPTION

In one aspect the present invention provides the use of a compound of formula

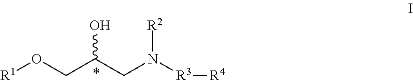

wherein
R¹ is a group of formula

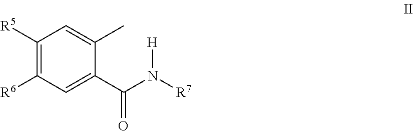

R² is hydrogen, (C₁₋₈)alkyl, or (C₃₋₆)cycloalkyl, wherein alkyl or cycloalkyl optionally are substituted by
(C₁₋₄)alkyl,
(C₁₋₄)alkoxy, or
phenyl, which phenyl optionally is substituted one or morefold, e.g. onefold by (C₁₋₆)alkoxy, e.g. (C₁₋₄)alkoxy,
   R³ is not present, or R³ is (C₁₋₈)alkylene, e.g. (C₁₋₆)alkylene, such as methylene, ethylene, propylene, butylene, isopentylene, which alkylene is unsubstituted, or substituted by (C₁₋₈)alkyl; preferably alkylene is unsubstituted, or
   R² and R³ together with the nitrogen atom to which they are attached form a heterocyclic ring, e.g. aliphatic, preferably comprising 6 or 7 ring members, such as 6 ring members, optionally comprising a further heteroatom, e.g. a further nitrogen atom, such as piperazinyl or piperidinyl,
   e.g. which heterocyclic ring optionally is substituted, e.g. R⁴ in that case is preferably benzhydryl, adamantanyl, benzyl or phenyl, wherein the phenyl group optionally is substituted by (C₁₋₄)alkyl,

### R⁴ is

hydrogen, if R³ is present,
(C₅₋₁₂)cycloalkyl, such as (C₈₋₁₂)cycloalkyl, e.g. cyclooctyl, cyclododecyl, adamantyl, such as adamantan-1-yl or adamantan-2-yl,
e.g. which cycloalkyl optionally is substituted, e.g. one or morefold, such as one or twofold, by (C₁₋₄)alkyl, e.g. methyl, or hydroxy,
(C₁₋₄)alkyl, such as methyl, optionally substituted by, e.g. one or more, such as one, phenyl, e.g. which phenyl optionally is substituted, e.g. by halo(C₁₋₄)alkyl, such as trifluoromethyl,
if R³ is present, R⁴ is
(C₆₋₁₂)aryl, e.g. phenyl, e.g. unsubstituted aryl or aryl one or morefold substituted, e.g. one or twofold, by
   (C₁₋₆)alkyl, (C₂₋₆)alkenyl, such as ethenyl, (C₂₋₆)alkynyl, (C₁₋₆)alkyloxy, such as methoxy, (C₂₋₆)alkenyl-(C₁₋₄)alkylene-oxy, HC≡C—(C₁₋₆)alkylene-oxy, such as propynyloxy, halogen, halogenated (C₁₋₄)alkyl, e.g. trifluoromethyl, (C₆₋₁₂)arylcarbonyl, e.g. phenylcarbonyl, or diazirinyl of formula

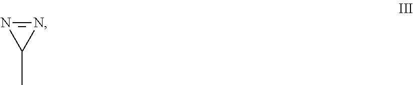

e.g. a group of formula

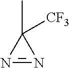

e.g. R⁴ is diazirinylphenyl of formula

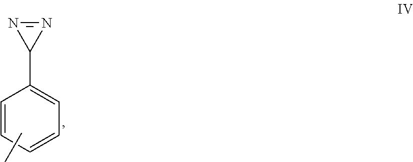

e.g. if R² and R³ together with the nitrogen atom to which they are attached form a heterocyclic ring, R⁴ is preferably (C₁₋₄)ₙ-alkylene-R^{7P}R^{8P}, wherein n is 0 or 1 and R^{7P} and R^{8P} are phenyl or hydrogen, wherein phenyl is unsubstituted or substituted by (C₁₋₄)alkyl, with the proviso that at least one of R^{7P} and R^{8P} is phenyl,
R⁵ and R⁶ independently of each other are hydrogen, halogen, e.g. Cl, Br, (C₁₋₄)alkyl, (C₂₋₄)alkenyl, (C₂₋₄)alkynyl, (C₁₋₄)alkoxy, (C₂₋₆)alkenyl-(C₁₋₄)alkylene-oxy, HC≡C—(C₁₋₆)alkylene-oxy, such as propynyloxy,
e.g. R₅ is hydrogen and R₆ is hydrogen, or is other than hydrogen and has the meaning as set out above, or
R⁵ and R⁶ together with the phenyl to which they are attached form an aromatic ring system, e.g. naphthalinyl,
R⁷ is (C₁₋₈)alkyl, such as methyl, ethyl, propyl, isopentyl, or (C₆₋₁₂)_{aryl}, e.g. phenyl, naphthalinyl, wherein alkyl is unsubstituted or substituted and aryl is substituted, e.g. one or morefold, e.g. one or twofold, by
halogen, such as bromo, fluoro.
(C₁₋₆)alkyl, e.g. tert-butyl, (C₂₋₄)alkenyl, (C₂₋₄)alkynyl,
halogenated (C₁₋₄)alkyl, e.g. CF₃,
(C₁₋₄)alkoxy, such as methoxy, (C₂₋₆)alkenyl-(C₁₋₄)alkylene-oxy, HC≡C—(C₁₋₆)alkylene-oxy,
(C₆₋₁₂)aryl, e.g. phenyl, naphthalinyl, which aryl, e.g. phenyl is unsubstituted or substituted, e.g. one or morefold, such as one or twofold by halogen, (C₁₋₄)alkyl, such as methyl, (C₂₋₆)alkenyl, such as allyl, (C₂₋₆)alkynyl, halogenated (C₁₋₄)alkyl, e.g. CF₃, (C₁₋₆)alkoxy, (C₂₋₆)alkenyl-(C₁₋₄)alkylenoxy, HC≡C—(C₁₋₆)alkylenoxy, phenylcarbonyl, or diazirinyl of formula III, e.g. a group of formula

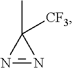

for use in the treatment of disorders, e.g. for the manufacture of a medicament for use in the treatment of disorders, mediated by protozoan organisms.

In a compound of formula I preferably
R² is hydrogen, methyl, propyl, e.g. n-propyl, butyl, e.g. n-butyl, pentyl, e.g. n-pentyl, isopentyl, methoxyphenyl-methyl, 2-phenylethyl, 3-phenyl-n-propyl or cyclopropyl,
R³ is not present, or R³ is methylene, ethylene, propylene, butylene or isopentylene, or R² and R³ together with the nitrogen atom to which they are attached form piperidinyl or piperazinyl; e.g. piperazin-1-yl or piperidin-1-yl, which is optionally substituted, e.g. in position 4 by R⁴, wherein R⁴ preferably is (C₁₋₄)ₙ-alkylene-R^{7P}R^{8P}, wherein n is 0 or 1 and R^{7P} and R^{8P} are phenyl or hydrogen, e.g. unsubstituted phenyl or phenyl substituted one or morefold by (C₁₋₄)alkyl, with the proviso that at least one of R^{7P} and R^{8P} is phenyl, e.g. piperidinyl, substituted by benzyl or phenyl, e.g. substituted phenyl, e.g. 2,3-dimethyl-phenyl,
e.g. piperazinyl substituted by benzhydryl or adamantanyl, e.g. adamantan-1-yl,
R⁴ is
   hydrogen, if R³ is present,
   (C₈₋₁₂)cycloalkyl, e.g. cyclooctyl, cyclododecyl, adamantyl, such as adamantan-1-yl or adamantan-2-yl, which (C₈₋₁₂)cycloalkyl optionally is substituted by (C₁₋₄)alkyl, e.g. methyl;
   methyl substituted by phenyl, e.g. substituted by substituted phenyl, e.g. trifluoromethyl-phenyl, such as 4-trifluoromethyl-phenyl, dimethylphenyl,
   ethenyl,
   admantanyl, e.g. adamantan-1-yl, adamantan-2-yl, e.g. which adamantanyl is substituted by hydroxy,
   cyclooctyl, cyclododecyl,
   unsubstituted phenyl, or phenyl substituted by
      (C₁₋₆)alkyl, phenylcarbonyl, e.g. 4-phenylcarbonyl, diazirinyl of formula III, e.g. a group of formula

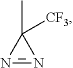

halogenated (C₁₋₄)alkyl, e.g. CF₃, (C₁₋₄)alkoxy, such as methoxy, HC≡C—(C₁₋₆)alkylenoxy, such as prop-2-ynyloxy,
e.g. phenyl, optionally substituted by methoxy, e.g. 4-methoxy, phenylcarbonyl, e.g. 4-phenylcarbonyl, a group of formula

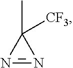

prop-2-ynyloxy,
R⁵ and R⁶ independently of each other are hydrogen, halogen, such as Cl, Br, or HC≡C—(C₁₋₆)alkylenoxy, such as prop-2-ynyloxy,
more preferably R⁵ is H and R⁶ is H, halogen, HC≡C—(C₁₋₆)alkylenoxy, such as prop-2-ynyloxy, or
R⁵ and R⁶ together with the phenyl to which they are attached form naphthalinyl,
R⁷ is
   (C₁₋₈)alkyl e.g. methyl, ethyl, propyl, pentyl, such as isopentyl, which alkyl is unsubstituted or alkyl substituted by
      phenyl, naphthalinyl, e.g. naphthalin-1-yl, (C₁₋₄)alkylphenyl, e.g. tert-butylphenyl, (C₁₋₄)alkoxyphenyl, e.g. methoxyphenyl, halogenated phenyl, e.g. fluorophenyl, bromophenyl, phenyl substituted by halogenated (C₁₋₄)alkyl, such as trifluoromethylphenyl, diazirinylphenyl, e.g. phenyl substituted with a group of formula

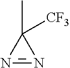

or (C₁₋₆)alkylphenyl, or
(C₆₋₁₂)aryl, e.g. naphthalinyl, or phenyl, wherein phenyl is substituted, e.g. one or twofold, by halogen, e.g. bromo, fluoro, or (C₁₋₆)alkyl, such as methyl, ethyl, propyl, isopentyl, butyl, e.g. tert-butyl, allyl, or halogenated (C₁₋₄)alkyl, e.g. CF₃.

In one particular aspect the present invention provides a compound of formula

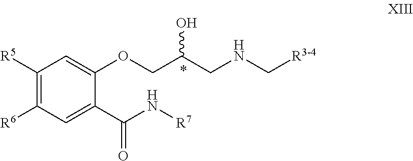

wherein
R³⁻⁴ is a diazirinylphenyl of formula IV, or 4-benzoylphenyl and R⁵, R⁶ and R⁷ are as defined above, e.g. useful as an intermediate in the preparation of compounds according to the present invention.

In still a further aspect the present invention provides a compound of formula I, which is a compound of formula

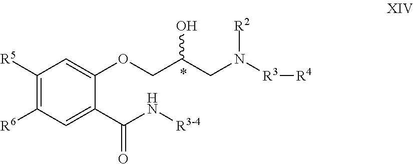

wherein
R³⁻⁴ is 4-benzoylphenyl or methyl, substituted by a diazirinylphenyl of formula

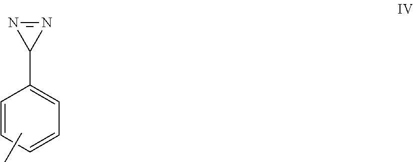

and R², R³, R⁴, R⁵ and R⁶ are as defined above, e.g. useful as an intermediate in the preparation of compounds according to the present invention.

In one preferred embodiment of the present invention in a compound of formula I R⁴ is (C₈₋₁₂)cycloalkyl, e.g. cyclooctyl, cyclododecyl, adamantyl, more preferably R⁴ is adamantyl, such as adamantan-1-yl or adamantan-2-yl, which (C₈₋₁₂)cycloalkyl optionally is substituted by (C₁₋₄)alkyl, e.g. methyl, hydroxy. Such compounds are novel and also form part of the present invention. Compounds of TABLE 1 wherein R⁴ is other than (C₈₋₁₂)cycloalkyl are novel in addition and also form part of the present invention.

In another aspect the present invention provides a compound of formula I, wherein R⁴ is (C₈₋₁₂)cycloalkyl, e.g. cyclooctyl, cyclododecyl, adamantyl, more preferably R⁴ is adamantyl, such as adamantan-1-yl or adamantan-2-yl, which (C₈₋₁₂)cycloalkyl optionally is substituted by (C₁₋₄)alkyl, e.g. methyl, hydroxy, such as (C₁₋₄)alkyl, and the other residues are as defined in a compound of formula I;

e.g. compounds of formula I wherein R⁴ is admantanyl of formulae I-1, I-2, I-3, I-4, I-5, I-6, 1-7, I-8, I-9, I-10, I-11, I-12, I-13, I-16, I-17, I-18, I-19, I-20, I-21, I-22, I-23, I-24, I-25, I-26, 1-27, I-28, I-29, I-30, I-31, I-32, I-33, 2 I-34, I-35, I-36, I-37, I-38, I-39, I-40, I-41, I-42, I-43, 1-55, I-62, I-64, I-66, I-70, I-71, I-72, I-73, I-74, I-75, I-76, I-86, I-87, I-91, I-92; and compounds of formula I wherein R⁴ is (C₅₋₁₂)cycloalkyl including adamantanyl, cyclooctyl and cyclododecyl; including the adamantanyl compounds indicated above and the compounds of formulae I-52 and I-53,
and for the case that in a compound of formula I as defined in claim 1 R⁴ is phenyl, additionally the compounds
2-{2-Hydroxy-3-[4-(3-trifluoromethyl-3H-diazirin-3-yl)-benzylamino]-propoxy}-N-(3-methyl-butyl)-5-prop-2-ynyloxy-benzamide of formula I-44,
N-(4-Fluoro-phenyl)-2-{2-hydroxy-3-[4-(3-trifluoromethyl-3H-diazirin-3-yl)-benzylamino]-propoxy}-5-prop-2-ynyloxy-benzamide of formula I-45,
2-[3-(4-Benzoyl-benzylamino)-2-hydroxy-propoxy]-N-benzyl-benzamide of formula I-49,
N-Benzyl-2-[2-hydroxy-3-(3-phenyl-propylamino)-propoxy]-benzamide of formula I-50,
2-(3-Benzylamino-2-hydroxy-propoxy)-N-(3-methyl-butyl)-benzamide of formula I-59,
2-[2-Hydroxy-3-(4-trifluoromethyl-benzylamino)-propoxy]-N-(3-methyl-butyl)-benzamide of formula I-60, and
N-Benzyl-2-{2-hydroxy-3-[(4-methoxy-3-prop-2-ynyloxy-benzyl)-propyl-amino]-propoxy}-benzamide of formula I-63,
   and for the case that in a compound of formula I as defined in claim 1 R² and R³ together with the nitrogen atom to which they are attached form a heterocyclic ring, additionally the compounds of formula

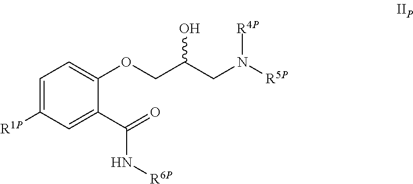

wherein
R^{1P} is hydrogen or halogen, preferably hydrogen,
R^{4P} and R^{5P} together with the nitrogen atom to which they are attached form piperidinyl or piperazinyl, which piperidinyl or piperazinyl optionally is substituted, e.g. in position 4, e.g. substituted by (C₁₋₄)ₙ-alkylene-R^{7P}R^{8P}, wherein n is 0 or 1 and R^{7P} and R^{8P} are phenyl or hydrogen, with the proviso that at least one of R^{7P} and R^{8P} is phenyl, e.g. piperidinyl or piperazinyl optionally are substituted by phenyl, benzyl or benzhydryl, e.g. wherein phenyl optionally is substituted by (C₁₋₄)alkyl, e.g. methyl, and
R^{6P} has the meaning of R^{3P} in formula Iₚ below, preferably R^{6P} is phenyl substituted by
halo(C₁₋₄)alkyl, e.g. R^{6P} is trifluoromethylphenyl, such as 3-trifluoromethylphenyl,
halogen, e.g. fluoro, e.g. one or two, R^{6P} is 2-fluorophenyl, 4-fluorophenyl, 2,4-di-fluorophenyl,
(C₁₋₄)alkyl, such as methyl, e.g. R^{6P} is 4-methylphenyl,
   including compounds of formulae I-46 to I-48, I-51, I-67 to I-69, I-77 to I-85, and I-88 to I-90, namely
N-(4-Fluoro-phenyl)-2-[2-hydroxy-3-(4-phenyl-piperidin-1-yl)-propoxy]-benzamide of formula I-46,
2-[3-(4-Benzyl-piperidin-1-yl)-2-hydroxy-propoxy]-N-(4-fluoro-phenyl)-benzamide of formula I-47,
2-[3-(4-Benzhydryl-piperazin-1-yl)-2-hydroxy-propoxy]-N-(4-fluoro-phenyl)-benzamide of formula I-48,
2-{3-[4-(2,3-Dimethyl-phenyl)-piperazin-1-yl]-2-hydroxy-propoxy}-N-(4-methoxy-benzyl)-benzamide of formula I-51,
2-[2-Hydroxy-3-(4-phenyl-piperidin-1-yl)-propoxy]-N-(3-trifluoromethyl-phenyl)-benzamide of formula I-67,
2-[3-(4-Benzyl-piperidin-1-yl)-2-hydroxy-propoxy]-N-(3-trifluoromethyl-phenyl)-benzamide of formula I-68,
2-[3-(4-Benzhydryl-piperazin-1-yl)-2-hydroxy-prop-oxy]-N-(3-trifluoromethyl-phenyl)-benzamide of formula I-69,
N-(2-Fluoro-phenyl)-2-[2-hydroxy-3-(4-phenyl-piperidin-1-yl)-propoxy]-benzamide of formula I-77,
2-[3-(4-Benzyl-piperidin-1-yl)-2-hydroxy-propoxy]-N-(2-fluoro-phenyl)-benzamide of formula I-78,
2-[3-(4-Benzhydryl-piperazin-1-yl)-2-hydroxy-propoxy]-N-(2-fluoro-phenyl)-benzamide benzamide of formula I-79,
2-[2-Hydroxy-3-(4-phenyl-piperidin-1-yl)-propoxy]-N-p-tolyl-benzamide of formula I-80,
2-[3-(4-Benzyl-piperidin-1-yl)-2-hydroxy-propoxy]-N-p-tolyl-benzamide of formula I-81,
2-[3-(4-Benzhydryl-piperazin-1-yl)-2-hydroxy-propoxy]-N-p-tolyl-benzamide of formula I-82,
5-Chloro-2-[2-hydroxy-3-(4-phenyl-piperidin-1-yl)-propoxy]-N-(3-trifluoromethyl-phenyl)-benzamide of formula I-83,
2-[3-(4-Benzyl-piperidin-1-yl)-2-hydroxy-propoxy]-5-chloro-N-(3-trifluoromethyl-phenyl)-benzamide of formula I-84,
2-[3-(4-Benzhydryl-piperazin-1-yl)-2-hydroxy-propoxy]-5-chloro-N-(3-trifluoromethyl-phenyl)-benzamide of formula I-85,
5-Bromo-N-(4-fluoro-phenyl)-2-[2-hydroxy-3-(4-phenyl-piperidin-1-yl)-propoxy]-benzamide of formula I-88,
2-[3-(4-Benzyl-piperidin-1-yl)-2-hydroxy-propoxy]-5-bromo-N-(4-fluoro-phenyl)-benzamide of formula I-89, and
2-[3-(4-Benzhydryl-piperazin-1-yl)-2-hydroxy-propoxy]-5-bromo-N-(4-fluoro-phenyl)-benzamide of formula I-90,
and for the case that in a compound of formula I as defined in claim 1 R³ is present and R⁴ is hydrogen, additionally the compounds
2-(2-Hydroxy-3-propylamino-propoxy)-N-p-tolyl-benzamide of formula I-54,
2-(3-Butylamino-2-hydroxy-propoxy)-N-(3-methyl-butyl)-benzamide of formula I-56,
2-(3-Dibutylamino-2-hydroxy-propoxy)-N-(3-methyl-butyl)-benzamide of formula I-57,
2-[2-Hydroxy-3-(3-methyl-butylamino)-propoxy]-N-(3-methyl-butyl)-benzamide of formula I-58,
2-(3-Dibutylamino-2-hydroxy-propoxy)-N-(4-fluoro-phenyl)-benzamide of formula I-61, and
2-(3-Allylamino-2-hydroxy-propoxy)-N-(2-allyl-phenyl)-benzamide of formula I-65.

In a still further aspect the present invention provides a compound of formula I, as defined above, selected from the group consisting of

2-[3-(Adamantan-1-ylamino)-2-hydroxy-propoxy]-N-benzyl-benzamide of formula I-1,
2-[3-(Adamantan-1-ylamino)-2-hydroxy-propoxy]-N-(2-methoxy-benzyl)-benzamide of formula I-2,
2-[3-(Adamantan-1-ylamino)-2-hydroxy-propoxy]-N-(4-fluoro-phenyl)-benzamide of formula I-3,
2-[3-(3,5-Dimethyl-adamantan-1-ylamino)-2-hydroxy-propoxy]-N-(4-fluoro-phenyl)-benzamide of formula I-4,
2-[3-(4-Adamantan-1-yl-piperazin-1-yl)-2-hydroxy-propoxy]-N-(4-fluoro-phenyl)-benzamide of formula I-5,
2-[3-(Adamantan-2-ylamino)-2-hydroxy-propoxy]-N-benzyl-benzamide of formula I-6,
2-[3-(Adamantan-2-ylamino)-2-hydroxy-propoxy]-N-(2-methoxy-benzyl)-benzamide of formula I-7,
2-[3-(Adamantan-2-ylamino)-2-hydroxy-propoxy]-N-(4-methoxy-benzyl)-benzamide of formula I-8,
2-[3-(Adamantan-2-ylamino)-2-hydroxy-propoxy]-N-phenethyl-benzamide of formula I-9,
2-[3-(Adamantan-2-ylamino)-2-hydroxy-propoxy]-5-chloro-N-phenethyl-benzamide of formula I-10,
2-[3-(Adamantan-2-ylamino)-2-hydroxy-propoxy]-N-(3-phenyl-propyl)-benzamide of formula I-11,
2-[3-(Adamantan-2-ylamino)-2-hydroxy-propoxy]-N-propyl-benzamide of formula I-12,
2-[3-(Adamantan-2-yl-methyl-amino)-2-hydroxy-propoxy]-N-benzyl-benzamide of formula I-13,
2-[3-(Adamantan-2-yl-methyl-amino)-2-hydroxy-propoxy]-N-(4-trifluoromethyl-benzyl)-benzamide of formula I-16,
2-[3-(Adamantan-2-yl-propyl-amino)-2-hydroxy-propoxy]-N-benzyl-benzamide of formula I-17,
2-[3-(Adamantan-2-yl-propyl-amino)-2-hydroxy-propoxy]-N-(2-methoxy-benzyl)-benzamide of formula I-18,
2-[3-(Adamantan-2-yl-pentyl-amino)-2-hydroxy-propoxy]-N-benzyl-benzamide of formula I-19,
2-[3-(Adamantan-2-yl-cyclopropyl-amino)-2-hydroxy-propoxy]-N-benzyl-benzamide of formula I-20,
2-{3-[Adamantan-2-yl-(2-methoxy-benzyl)-amino]-2-hydroxy-propoxy}-N-benzyl-benzamide of formula I-21,
2-[3-(Adamantan-2-yl-phenethyl-amino)-2-hydroxy-propoxy]-N-benzyl-benzamide of formula I-22,
2-{3-[Adamantan-2-yl-(3-phenyl-propyl)-amino]-2-hydroxy-propoxy}-N-benzyl-benzamide of formula I-23,
2-{3-[(Adamantan-1-ylmethyl)-amino]-2-hydroxy-propoxy}-N-(3-methyl-butyl)-benzamide of formula I-24,
2-[3-(1-Adamantan-1-yl-ethylamino)-2-hydroxy-propoxy]-N-(3-methyl-butyl)-benzamide of formula I-25,
2-{3-[(Adamantan-1-ylmethyl)-amino]-2-hydroxy-propoxy}-N-(3-methyl-butyl)-5-prop-2-ynyloxy-benzamide of formula I-26,
2-{3-[(Adamantan-1-ylmethyl)-amino]-2-hydroxy-propoxy}-N-benzyl-benzamide of formula I-27,
2-[3-(1-Adamantan-1-yl-ethylamino)-2-hydroxy-propoxy]-N-benzyl-benzamide of formula I-28,
2-{3-[(Adamantan-1-ylmethyl)-amino]-2-hydroxy-propoxy}-N-(2-methoxy-benzyl)-benzamide of formula I-29,
2-{3-[(Adamantan-1-ylmethyl)-amino]-2-hydroxy-propoxy}-N-(4-trifluoromethyl-benzyl)-benzamide of formula I-30,
2-{3-[(Adamantan-1-ylmethyl)-amino]-2-hydroxy-propoxy}-N-phenethyl-benzamide of formula I-31,
2-{3-[(Adamantan-1-ylmethyl)-amino]-2-hydroxy-propoxy}-N-(3-phenyl-propyl)-benzamide of formula I-32,
2-{3-[(Adamantan-1-ylmethyl)-amino]-2-hydroxy-propoxy}-5-chloro-N-phenethyl-benzamide of formula I-33,
2-{3-[(Adamantan-1-ylmethyl)-amino]-2-hydroxy-propoxy}-5-chloro-N-naphthalen-1-ylmethyl-benzamide of formula I-34,
2-[3-(1-Adamantan-1-yl-ethylamino)-2-hydroxy-propoxy]-5-chloro-N-naphthalen-1-ylmethyl-benzamide of formula I-35,
2-[3-(Adamantan-1-ylmethyl-methyl-amino)-2-hydroxy-propoxy]-N-benzyl-benzamide of formula I-36,
2-[3-(Adamantan-1-ylmethyl-methyl-amino)-2-hydroxy-propoxy]-N-(4-fluoro-phenyl)-benzamide of formula I-37,
2-{3-[(Adamantan-1-ylmethyl)-amino]-2-hydroxy-propoxy}-N-(2-fluoro-phenyl)-benzamide of formula I-38,
2-{3-[(Adamantan-1-ylmethyl)-amino]-2-hydroxy-propoxy}-N-(2,4-difluoro-phenyl)-benzamide of formula I-39,
2-{3-[(Adamantan-1-ylmethyl)-amino]-2-hydroxy-propoxy}-N-p-tolyl-benzamide of formula I-40,
2-[3-(1-Adamantan-1-yl-ethylamino)-2-hydroxy-propoxy]-N-p-tolyl-benzamide of formula I-41,
2-{3-[(Adamantan-1-ylmethyl)-amino]-2-hydroxy-propoxy}-N-(3-trifluoromethyl-phenyl)-benzamide of formula I-42,
2-[3-(Adamantan-2-yl-methyl-amino)-2-hydroxy-propoxy]-N-[4-(3-trifluoromethyl-3H-diazirin-3-yl)-benzyl]-benzamide of formula I-43,
2-{2-Hydroxy-3-[4-(3-trifluoromethyl-3H-diazirin-3-yl)-benzylamino]-propoxy}-N-(3-methyl-butyl)-5-prop-2-ynyloxy-benzamide of formula I-44,
N-(4-Fluoro-phenyl)-2-{2-hydroxy-3-[4-(3-trifluoromethyl-3H-diazirin-3-yl)-benzylamino]-propoxy}-5-prop-2-ynyloxy-benzamide of formula I-45,
N-(4-Fluoro-phenyl)-2-[2-hydroxy-3-(4-phenyl-piperidin-1-yl)-propoxy]-benzamide of formula I-46,
2-[3-(4-Benzyl-piperidin-1-yl)-2-hydroxy-propoxy]-N-(4-fluoro-phenyl)-benzamide of formula I-47,
2-[3-(4-Benzhydryl-piperazin-1-yl)-2-hydroxy-propoxy]-N-(4-fluoro-phenyl)-benzamide of formula I-48,
2-[3-(4-Benzoyl-benzylamino)-2-hydroxy-propoxy]-N-benzyl-benzamide of formula I-49,
N-Benzyl-2-[2-hydroxy-3-(3-phenyl-propylamino)-propoxy]-benzamide of formula I-50,
2-{3-[4-(2,3-Dimethyl-phenyl)-piperazin-1-yl]-2-hydroxy-propoxy}-N-(4-methoxy-benzyl)-benzamide of formula I-51,
N-Benzyl-2-(3-cyclododecylamino-2-hydroxy-propoxy)-benzamide of formula I-52,
N-Benzyl-2-(3-cyclooctylamino-2-hydroxy-propoxy)-benzamide of formula I-53,
2-(2-Hydroxy-3-propylamino-propoxy)-N-p-tolyl-benzamide of formula I-54,
2-{3-[(Adamantan-1-ylmethyl)-amino]-2-hydroxy-propoxy}-N-(4-fluoro-phenyl)-benzamide of formula I-55,
2-(3-Butylamino-2-hydroxy-propoxy)-N-(3-methyl-butyl)-benzamide of formula I-56,
2-(3-Dibutylamino-2-hydroxy-propoxy)-N-(3-methyl-butyl)-benzamide of formula I-57,
2-[2-Hydroxy-3-(3-methyl-butylamino)-propoxy]-N-(3-methyl-butyl)-benzamide of formula I-58,
2-(3-Benzylamino-2-hydroxy-propoxy)-N-(3-methyl-butyl)-benzamide of formula I-59,
2-[2-Hydroxy-3-(4-trifluoromethyl-benzylamino)-propoxy]-N-(3-methyl-butyl)-benzamide of formula I-60,
2-(3-Dibutylamino-2-hydroxy-propoxy)-N-(4-fluoro-phenyl)-benzamide of formula I-61,
2-[3-(2-Adamantan-1-yl-ethylamino)-2-hydroxy-propoxy]-N-benzyl-benzamide of formula I-62,
N-Benzyl-2-{2-hydroxy-3-[(4-methoxy-3-prop-2-ynyloxy-benzyl)-propyl-amino]-propoxy}-benzamide of formula I-63,
2-[3-(Adamantan-2-yl-methyl-amino)-2-hydroxy-propoxy]-N-(4-tert-butyl-benzyl)-benzamide of formula I-64,
2-(3-Allylamino-2-hydroxy-propoxy)-N-(2-allyl-phenyl)-benzamide of formula I-65,
2-{3-[(Adamantan-1-ylmethyl)-amino]-2-hydroxy-pro-poxy}-5-chloro-N-(4-fluoro-phenyl)-benzamide of formula I-66,
2-[2-Hydroxy-3-(4-phenyl-piperidin-1-yl)-propoxy]-N-(3-trifluoromethyl-phenyl)-benzamide of formula I-67,
2-[3-(4-Benzyl-piperidin-1-yl)-2-hydroxy-propoxy]-N-(3-trifluoromethyl-phenyl)-benzamide of formula I-68,
2-[3-(4-Benzhydryl-piperazin-1-yl)-2-hydroxy-prop-oxy]-N-(3-trifluoromethyl-phenyl)-benzamide of formula I-69,
2-[2-Hydroxy-3-(3-hydroxy-adamantan-1-ylamino)-propoxy]-N-(3-trifluoromethyl-phenyl)-benzamide of formula I-70,
2-{3-[(Adamantan-1-ylmethyl)-amino]-2-hydroxy-propoxy}-N-naphthalen-2-yl-benzamide of formula I-71,
2-{3-[(Adamantan-1-ylmethyl)-amino]-2-hydroxy-propoxy}-5-chloro-N-(3-trifluoromethyl-phenyl)-benzamide of formula I-72,
2-{3-[(Adamantan-1-ylmethyl)-amino]-2-hydroxy-propoxy}-N-(2-allyl-phenyl)-benzamide of formula I-73,
2-{3-[(Adamantan-1-ylmethyl)-amino]-2-hydroxy-propoxy}-N-(4-bromo-phenyl)-benzamide of formula I-74,
2-[3-(Adamantan-1-ylamino)-2-hydroxy-propoxy]-N-(4-bromo-phenyl)-benzamide of formula I-75,
N-(4-Bromo-phenyl)-2-[2-hydroxy-3-(3-hydroxy-adamantan-1-ylamino)-propoxy]-benzamide of formula I-76,
N-(2-Fluoro-phenyl)-2-[2-hydroxy-3-(4-phenyl-piperidin-1-yl)-propoxy]-benzamide of formula I-77,
2-[3-(4-Benzyl-piperidin-1-yl)-2-hydroxy-propoxy]-N-(2-fluoro-phenyl)-benzamide of formula I-78,
2-[3-(4-Benzhydryl-piperazin-1-yl)-2-hydroxy-propoxy]-N-(2-fluoro-phenyl)-benzamide of formula I-79,
2-[2-Hydroxy-3-(4-phenyl-piperidin-1-yl)-propoxy]-N-p-tolyl-benzamide of formula I-80,
2-[3-(4-Benzyl-piperidin-1-yl)-2-hydroxy-propoxy]-N-p-tolyl-benzamide of formula I-81,
2-[3-(4-Benzhydryl-piperazin-1-yl)-2-hydroxy-propoxy]-N-p-tolyl-benzamide of formula I-82,
5-Chloro-2-[2-hydroxy-3-(4-phenyl-piperidin-1-yl)-propoxy]-N-(3-trifluoromethyl-phenyl)-benzamide of formula I-83,
2-[3-(4-Benzyl-piperidin-1-yl)-2-hydroxy-propoxy]-5-chloro-N-(3-trifluoromethyl-phenyl)-benzamide of formula I-84,
2-[3-(4-Benzhydryl-piperazin-1-yl)-2-hydroxy-propoxy]-5-chloro-N-(3-trifluoromethyl-phenyl)-benzamide of formula I-85,
3-{3-[(Adamantan-1-ylmethyl)-amino]-2-hydroxy-propoxy}-naphthalene-2-carboxylic acid (4-fluoro-phenyl)-amide of formula I-86,
3-[3-(Adamantan-1-ylamino)-2-hydroxy-propoxy]-naphthalene-2-carboxylic acid (4-fluoro-phenyl)-amide of formula I-87,
5-Bromo-N-(4-fluoro-phenyl)-2-[2-hydroxy-3-(4-phenyl-piperidin-1-yl)-propoxy]-benzamide of formula I-88,
2-[3-(4-Benzyl-piperidin-1-yl)-2-hydroxy-propoxy]-5-bromo-N-(4-fluoro-phenyl)-benzamide of formula I-89,
2-[3-(4-Benzhydryl-piperazin-1-yl)-2-hydroxy-propoxy]-5-bromo-N-(4-fluoro-phenyl)-benzamide of formula I-90,
2-{3-[(Adamantan-1-ylmethyl)-amino]-2-hydroxy-propoxy}-5-bromo-N-(4-fluoro-phenyl)-benzamide of formula I-91, and
2-[3-(Adamantan-1-ylamino)-2-hydroxy-propoxy]-5-bromo-N-(4-fluoro-phenyl)-benzamide of formula I-92.

In a preferred embodiment the present invention provides a compound of formula

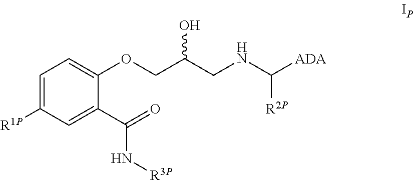

wherein ADA is adamantyl, e.g. adamant-1-yl or adamant-2-yl, which adamantyl optionally is substituted by (C₁₋₄)alkyl, or hydroxy,
R^{1P} is hydrogen or halogen, e.g. chloro, bromo, such as chloro,

### R^{2P} is

hydrogen,
(C₁₋₈)alkyl, such as (C₁₋₆)alkyl, e.g. unsubstituted alkyl, such as methyl, propyl, pentyl, or alkyl substituted by
   phenyl, e.g. unsubstituted phenyl, or substituted phenyl, e.g. phenyl substituted by (C₁₋₄)alkoxy, e.g. methoxy,
(C₃₋₆)cycloalkyl, such as cyclopropyl, and

### R^{3P} is

(C₆₋₁₂)aryl, e.g. phenyl or naphthalinyl, which aryl is unsubstituted or substituted, including e.g. aryl substituted by one or more, e.g. one or two
   halogen, e.g. fluoro, bromo,
   (C₁₋₄)alkyl, e.g. methyl,
   (C₂₋₄)alkenyl, e.g. allyl,
   halo(C₁₋₄)alkyl, e.g. CF₃, or
   (C₁₋₄)alkoxy, such as methoxy,
e.g. in particular phenyl is optionally substituted and naphthalinyl is unsubstituted,
or
   (C₁₋₁₂)alkyl, e.g. which alkyl is unsubstituted, e.g. in which case alkyl is preferably (C₃₋₈)alkyl, e.g. propyl, isopentyl, or which alkyl is substituted by (C₆₋₁₂)aryl, e.g. phenyl or naphthalinyl, in which case alkyl is preferably (C₁₋₆)alkyl, e.g. methyl, ethyl, propyl, wherein aryl is unsubstituted or substituted, e.g. substituted by a group of formula

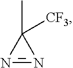

or substituted as substituted aryl in the meaning of R^{3P},
e.g. compounds of formulae I-1 to I-43, I-55, I-62, I-64, I-66, I-70 to I-76, I-86, I-87, I-91 and I-92.

In another preferred embodiment the present invention provides a compound of formula I, which is a compound of formula

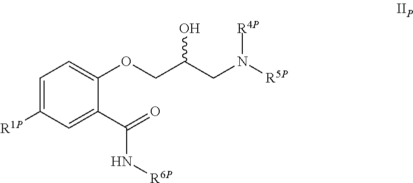

wherein
R^{1P} is hydrogen or halogen, preferably hydrogen,
R^{4P} and R^{5P} together with the nitrogen atom to which they are attached form piperidinyl or piperazinyl, which piperidinyl or piperazinyl is substituted, e.g. in position 4, e.g. substituted by (C₁₋₄)ₙ-alkylene-R^{7P}R^{8P}, wherein n is 0 or 1 and R^{7P} and R^{8P} are phenyl or hydrogen, with the proviso that at least one of R^{7P} and R^{8P} is phenyl, e.g. piperidinyl or piperazinyl optionally are substituted by phenyl, benzyl or benzhydryl, e.g. wherein phenyl optionally is substituted by (C₁₋₄)alkyl, e.g. methyl, and
R^{6P} has the meaning of R^{3P}, preferably R^{6P} is phenyl substituted by
halo(C₁₋₄)alkylphenyl, e.g. trifluoromethylphenyl, such as 2-trifluoromethylphenyl,
halogen, e.g. fluoro, e.g. 2-fluorophenyl,
(C₁₋₄)alkyl, such as methyl, including 4-methylphenyl,
   including compounds of formulae I-46 to I-48, I-67 to I-69, I-77 to I-85, and I-88 to I-90.

In another aspect a compound of the present invention is selected from the compounds of formulae I-39, I-40, I-41, I-42, I-55, I-66, I-67, I-68, I-69, I-71, I-72, I-74, I-84 and I-85.

Novel compounds provided by the present invention are herein also designated as “compound(s) of (according to) the present invention.” Active compounds of the present invention include the compounds of the present invention.

In one particular embodiment of the present invention in a compound of formula I R⁶ preferably is other than H.

In a further aspect the present invention provides the compound of formula I-1 to I-13 and I-16 to I-92 as set out in TABLE 1 in the example part, which compounds are compounds of formula I. The compounds of formula INT-14 and INT-15 in TABLE I are intermediates for the preparation of compounds of formula I. Characterization data of compounds of formula I-1 to I-13 and I-16 to I-92 and INT-14 and INT-15 are also set out in TABLE 1.

In a compound of formula I each single group of substituents defined may be a preferred group of substituents, e.g. independently of each other group of substituents or single substituents defined.

If not specifically otherwise defined herein

any group (substituent) defined herein may comprise 1 to 18 carbon atoms, for example
alkyl—including the part “alk” in other groups like alkoxy—includes (C₁₋₁₂)alkyl, e.g. (C₁₋₈)alkyl, such as (C₁₋₄)alkyl;
alkenyl—including the part “alkenyl” in other groups like alkenylalkylenoxy—includes (C₂₋₁₂)alkenyl, e.g. (C₂₋₆)alkenyl, such as (C₂₋₄)alkenyl;
alkynyl includes (C₂₋₁₂)alkynyl, e.g. (C₂₋₆)alkynyl, such as (C₂₋₄)alkynyl;
cycloalkyl includes (C₃₋₁₂)cycloalkyl, e.g. (C₃₋₆)cycloalkyl, e.g. (C₈₋₁₂)cycloalkyl,
aryl includes (C₆₋₁₈)aryl, e.g. phenyl, naphthyl, phenanthrenyl, e.g. phenyl, naphthyl,
acyl includes (C₁₋₁₂)acyl, e.g. including alkylcarbonyl, alkenylcarbonyl, alkynylcarbonyl, cycloalkylcarbonyl, arylcarbonyl and heterocyclylcarbonyl,
heterocyclyl e.g. includes
   aliphatic heterocyclyl and aromatic heterocyclyl,
   4 to 8 membered heterocyclyl,
   heterocyclyl optionally anellated with another ring (system), e.g. anellated with aryl, e.g. anellated with a heterocyclic ring (system);
   heterocyclyl having 1 to 4 heteroatoms selected from S, O, N;
amine includes unsubstituted amine and amine substituted by alkyl, cycloalkyl, aryl, heterocyclyl;
halogen includes fluoro, chloro, bromo.

Any group (compound) defined herein may be unsubstituted or substituted, e.g. onefold or morefold, e.g. onefold, twofold.

Compounds provided by the present invention and compounds which are useful according to the present invention are hereinafter designated also as “active compound(s) of (according to) the present invention”. An active compound of the present invention includes a compound in any form, e.g. in free form and in the form of cocrystals, such as in the form of a salt, in the form of a solvate and in the form of a salt and a solvate.

In another aspect the present invention provides an active compound of the present invention in the form of a salt.

Such salts include preferably pharmaceutically acceptable salts, although pharmaceutically unacceptable salts are included, e.g. for preparation/isolation/purification purposes.

A salt of an active compound of the present invention includes a metal salt or an acid addition salt.

In another aspect the present invention provides an active compound of the present invention in the form of a salt.

Such salts include preferably pharmaceutically acceptable salts, although pharmaceutically unacceptable salts are included, e.g. for preparation/isolation/purification purposes.

A salt of an active compound of the present invention includes a metal salt or an acid addition salt.

An active compound of the present invention in free form may be converted into a corresponding compound in the form of a salt; and vice versa. A compound of the present invention in free form or in the form of a salt and in the form of a solvate may be converted into a corresponding compound in free form or in the form of a salt in non-solvated form; and vice versa.

An active compound of the present invention and optionally an intermediate in its preparation, may exist in the form of isomers and mixtures thereof; e.g. optical isomers, diastereoisomers, cis/trans isomers. An active compound of the present invention may e.g. contain asymmetric carbon atoms and may thus exist in the form of enantiomers or diastereoisomers and mixtures thereof, e.g. racemates. A compound of the present invention may be present in the (R)-, (S)- or (R,S)-configuration preferably in the (R)- or (S)-configuration regarding each of the substituents at such asymmetric carbon atoms in an active compound of the present invention. For example, an active compound of the present invention may be present in the (R)-, (S)- or (R,S)-configuration preferably in the (R)- or (S)-configuration regarding the hydroxy group in a compound of formula I which is attached to an asymmetric carbon atom.

Isomeric mixtures may be separated as appropriate, e.g. according, e.g. analogously, to a method as conventional, to obtain pure isomers. The present invention includes an active compound of the present invention in any isomeric form and in any isomeric mixture.

The present invention also includes tautomers of an active compound of the present invention, where tautomers can exist.

The synthesis of an active compound of formula I of the present invention may be carried out according to the following REACTION SCHEME 1 or REACTION SCHEME 2:

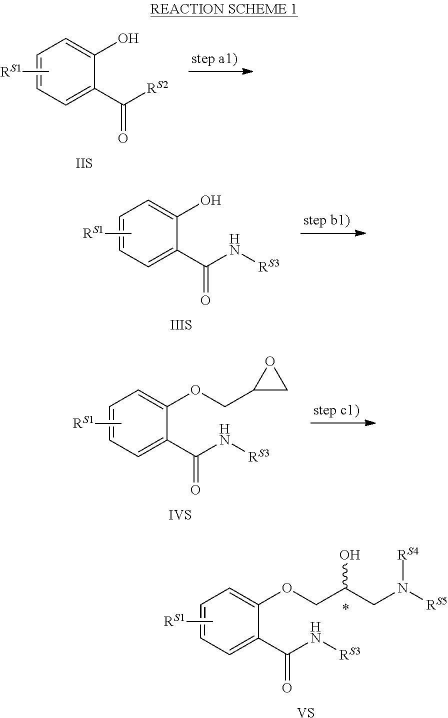

In REACTION SCHEME 1 the salicylates of formula IIS, e.g. methyl- or phenylesters which are known compounds, may serve as starting materials. From salicylates of formula IIS salicylic acid amides of formula HIS are accessible by numerous methods described in chemical literature. The aminoalcohol motif in a compound of formula VS may be established by O-alkylation of 2-hydroxybenzamides of formula HIS with epichlorohydrin to give a compound of formula IVS. Subsequent nucleophilic ring opening of the oxirane in a compound of formula IVS with an amine nucleophile affords a compound of formula VS. The principles of the reaction steps a1) to c1) are well known in chemistry.

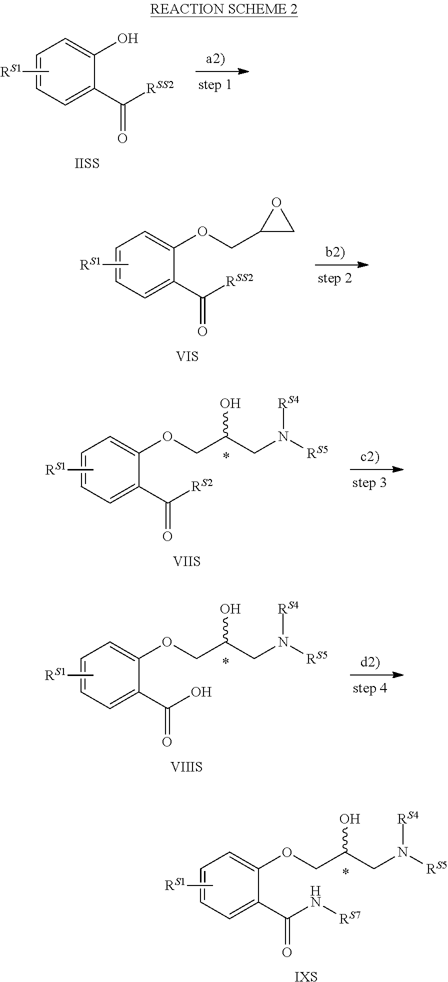

In REACTION SCHEME 2 the salicylates of formula IISS, e.g. methyl- or ethylesters which are known compounds, may serve as starting materials. From the salicylates of formula IISS the oxiranes of formula VIS may be prepared in step a2) by reaction with epichlorohydrin. The oxiranes of formula VIS are subjected to nucleophilic ring opening by reaction with an amine in step b2) and the aminoalcohols of formula VIIS are obtained. Saponification of esters of formula VIIS in step c2) affords carboxylic acids of formula VIIIS, which are reacted with an amine in step d2) to obtain a compound of formula IXS. The principles of reaction steps a2) to d2) are well known in chemistry.

In another aspect the present invention provides a process for the production of a compound of formula I, e.g. including an active compound of the present invention and a compound of the present invention, wherein R¹, R², R³, R⁴, R⁵, R⁶ and R⁷ are as defined above, comprising the steps of

either
a1) reacting a compound of formula

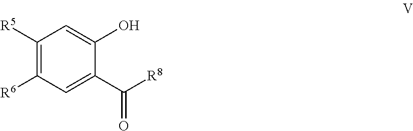

wherein R⁵ and R⁶ are as defined above and R⁸ is methoxy or phenoxy, with an amine of formula

H₂N—R⁷ VI
wherein R⁷ is as defined above to obtain a compound of formula

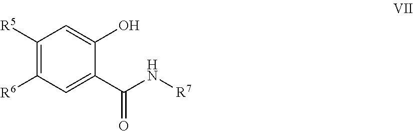

wherein R⁵, R⁶ and R⁷ are as defined above,
b1) reacting a compound of formula VII with epichlorohydrin to give a compound of formula

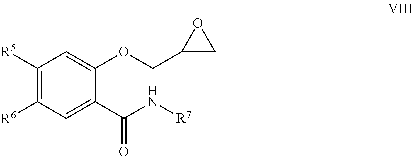

wherein R⁵, R⁶ and R⁷ are as defined above,
c1) ring opening of the oxirane ring in a compound of formula VIII with an amine of formula

NH(R²)(R³—R⁴) IX
wherein R², R³ and R⁴ are as defined above,
   or
a2) reacting a compound of formula

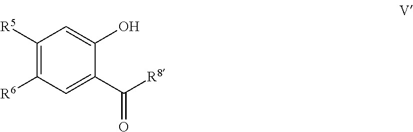

wherein R⁵ and R⁶ are as defined above and R^{8′} is methoxy or ethoxy, with epichlorohydrin to give a compound of formula

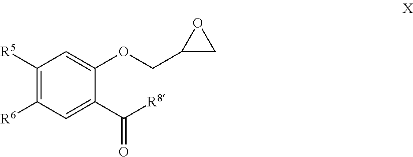

wherein R⁵, R⁶ and R^{8′} are as defined above,
b2) ring opening of the oxirane ring in a compound of formula X with an amine of formula

NH(R²)(R³—R⁴) IX
wherein R², R³ and R⁴ are as defined above, to obtain a compound of formula

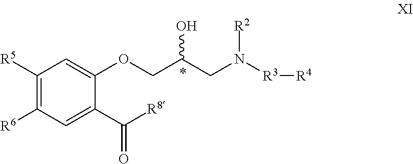

wherein R², R³, R⁴ R⁵, R⁶ and R^{8′} are as defined above,
c2) saponifying a compound of formula XI, wherein R², R³, R⁴ R⁵, R⁶ and R^{8′} are as defined above to obtain a compound of formula

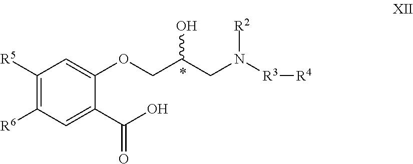

wherein R², R³, R⁴, R⁵ and R⁶ are as defined above; and
d2) reacting a compound of formula XII, wherein R², R³, R⁴, R⁵ and R⁶ are as defined above with an amine of formula

H₂N—R⁷ VI
wherein R⁷ is as defined above, and
   isolating a compound of formula I, wherein R¹, R², R³, R⁴, R⁵, R⁶ and R⁷ are as defined above from the reaction mixture.

A compound of formula I thus obtained may be converted into another compound of formula I. A compound of formula I obtained in free form may be converted into a salt of a compound of formula I, or, vice versa, a compound of formula I in the form of a salt may be converted into a compound of formula I in free form.

In an intermediate of formula V, V′, VI, VII, VIII, IX, X, XI or XII (starting materials), functional groups, if present, optionally may be in protected form or in the form of a salt, if a salt-forming group is present. Protecting groups, optionally present, may be removed at an appropriate stage, e.g. according, e.g. analogously, to a method as conventional.

Epichlorohydrin in step b1) or a2) may be used as a racemic (R,S) mixture or in enantiomerically pure form, either as (S)-2-chloromethyl-oxirane or (R)-2-chloromethyl-oxirane. Such reagents are commercially available. (R,S)-epichlorohydrin has been used for synthesis of the compounds of the present invention. The stereochemistry on the hydroxy substituted carbon (C* in REACTION SCHEME 1) of the aminoalcohol substructure can be controlled by employing either (R)- or (S)-epichlorohydrin in the O-alkylation step. All final racemic compounds of formula I can therefore be synthesized as single (R)- or (S)-enantiomers regarding the hydroxy group following the same synthetic procedures.

Secondary salicylic acid amides of formula VII are preferred starting materials for the synthesis of a compound of formula VIII. Salicylates of formula V or V′ which are substituted in 5-position, preferably by halogen or alkoxy, are preferred for the synthesis of amides of formula VIII.

The oxirane intermediates of formula VIII obtainable by O-alkylation of the secondary salicylic acid amides of formula VII, or the oxirane intermediates of formula X, obtainable by O-alkylation of a compound of formula V may be subjected to nucleophilic ring opening with primary or secondary amines Preferred amines for epoxide ring opening are amine building blocks substituted with adamantyl residues.

Ring opening of the oxirane ring in a compound of formula VIII or X, respectively may be carried out with non-adamantyl substituted amines, e.g. to obtain compounds of formula I (44-54, 56-61, 63).

Ring opening of the oxirane ring in a compound of formula VIII or X, respectively may be carried out with non-adamantyl substituted amines, e.g. to obtain compounds of formula I of formulae I-44 to I-54 (including I-52 and I-53), I-56 to I-61, I-63, I-65, I-67 to I-68, I-77 to I-85).

In the synthesis according to REACTION SCHEME 1 in step a1) and according to REACTION SCHEME 2 in step d2) amines are used as reagents. Preferred amines for amide synthesis are primary aromatic amines preferably additionally substituted on the aromatic ring by electron withdrawing groups such as fluorine or trifluoromethyl groups.

The above reactions in step a1) and in step d2), respectively, are amidation reactions of carboxylic acid derivatives and may be carried out as appropriate, e.g. analogously to a method as conventional.

The above reactions in step b1) and step a2), respectively, are alkylation reactions of alcohol derivatives and may be carried out as appropriate, e.g. analogously to a method as conventional.

The above reactions in step c1) and in step b2) are oxirane ring opening reactions with an amine and may be carried out as appropriate, e.g. analogously to a method as conventional. The above reaction step c2) is a saponification of a carboxylic acid ester and may be carried out as appropriate, e.g. analogously to a method as conventional.

Intermediates (starting materials) of V, V′, VI, VII, VIII, IX, X, XI or XII are known or may be prepared according, e.g. analogously, to a method as conventional or as specified herein.

E.g. the compound 2-oxiranylmethoxy-benzoic acid methyl ester, e.g. useful for the preparation of a compound of formula I-43 according to REACTION SCHEME 2, is a known compound (CAS 22589-46-4).

For example, a compound of formula V′, wherein R⁵ and/or R⁶ is alkoxy, alkenyl-alkylenoxy, HC≡C-alkylenoxy, respectively, may be prepared from a compound of formula

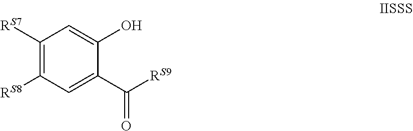

wherein R^{S9} is methoxy or ethoxy and R^{S7} and/or R^{S8} independently of each other are hydroxy by alkylation with a corresponding alkyl-, alkenyl- or alkynyl-halogenide. Compounds of formula IISSS wherein R^{S9}, R^{S7} and R^{S8} are as defined above are known.

Certain compounds of formula VIII, wherein R⁵, R⁶ and R⁷ are as defined above are novel, e.g. and of formula XI, wherein R², R³, R⁴, R⁵, R⁶ and R^{8′} are as defined above, are novel which novel compounds also form part of the present invention.

In another aspect the present invention provides a compound selected from
5-Chloro-2-hydroxy-N-naphthalen-1-ylmethyl-benzamide,
N-(4-Fluoro-phenyl)-2-hydroxy-5-prop-2-ynyloxy-benzamide,
N-(2-Methoxy-benzyl)-2-oxiranylmethoxy-benzamide,
N-(4-Methoxy-benzyl)-2-oxiranylmethoxy-benzamide,
2-Oxiranylmethoxy-N-phenethyl-benzamide,
5-Chloro-2-oxiranylmethoxy-N-phenethyl-benzamide,
2-(Oxiran-2-ylmethoxy)-N-(3-phenylpropyl)benzamide,
N-(3-Methyl-butyl)-2-oxiranylmethoxy-benzamide,
2-Oxiranylmethoxy-N-(4-trifluoromethyl-benzyl)-benzamide,
5-Chloro-N-naphthalen-1-ylmethyl-2-oxiranylmethoxy-benzamide,
N-(4-Fluoro-phenyl)-2-oxiranylmethoxy-benzamide,
N-(2-Fluoro-phenyl)-2-oxiranylmethoxy-benzamide,
N-(2,4-Difluoro-phenyl)-2-oxiranylmethoxy-benzamide,
2-Oxiranylmethoxy-N-(3-trifluoromethyl-phenyl)-benzamide, and
N-(4-Fluoro-phenyl)-2-oxiranylmethoxy-5-prop-2-ynyloxy-benzamide,
N-(2-Allyl-phenyl)-2-oxiranylmethoxy-benzamide,
5-Chloro-N-(4-fluoro-phenyl)-2-oxiranylmethoxy-benzamide,
5-Chloro-2-oxiranylmethoxy-N-(3-trifluoromethyl-phenyl)-benzamide,
N-Naphthalen-2-yl-2-oxiranylmethoxy-benzamide,
N-(2-Allyl-phenyl)-2-hydroxy-benzamide,
2-[3-(Adamantan-2-yl-methyl-amino)-2-hydroxy-propoxy]-benzoic acid methyl ester (INT-14),
2-[3-(Adamantan-2-yl-methyl-amino)-2-hydroxy-propoxy]-benzoic acid (INT-15).
   e.g.
2-[3-(Adamantan-2-yl-methyl-amino)-2-hydroxy-propoxy]-benzoic acid methyl ester (INT-14),
2-[3-(Adamantan-2-yl-methyl-amino)-2-hydroxy-propoxy]-benzoic acid (INT-15).

Another valuable intermediate in the preparation of a compound of formula I is a compound of formula

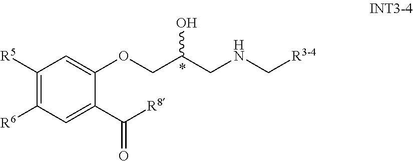

wherein R³⁻⁴ is a diazirinylphenyl of formula IV, or benzoylphenyl, such as 4-benzoylphenyl and R^{8′} is as defined above; which intermediate of formula INT3-4 also forms part of the present invention.

Compounds of formula VIII and XI, wherein the residues are defined as above, are useful intermediates for the preparation of a compound of formula I.

Any compound described herein, e.g. a compound of the present invention and intermediates of formula V, V′, VI, VII, VIII, IX, X, XI or XII may be prepared as appropriate, e.g. according, e.g. analogously, to a method as conventional, e.g. or as specified herein.

The compounds of the present invention, e.g. including a compound of formula I, exhibit pharmacological activity and are therefore useful as pharmaceuticals. E.g., the compounds of the present invention are found to inhibit growth of protozoan organisms, such as apicomplexa, kinetoplastids, microsporidia, plasmodia. Numerous diseases, such as malaria, Chagas disease, sleeping sickness, trichomoniasis, leishmaniasis, giardiasis, amebiasis, toxoplasmosis, trypanosomiasis (animal sleeping sickness), babesiosis, theileriosis, coccidiosis are mediated, e.g. caused, by infection with protozoan organisms. Plasmodium organisms are responsible for malaria the most prevalent disease in South Asia and sub-Saharan Africa.

### Activity Testing

### Assessment of Antimalarial Potency (IC₅₀)

Highly sensitive HRP2-based ELISA and [³H]-hypoxanthine incorporation assay were used as markers for inhibition of parasite growth as previously described (12, 14). A solvent control and a chloroquine (CQ) control were included in the HRP2-based ELISA. CQ, lumefantrine and artesunate were included as controls in the [³H]-hypoxanthine assay series.

The assay protocol of the HRP2-based ELISA is described in more detail in the following paragraphs.

The P. falciparum clones were maintained in continuous culture as described previously (10). Briefly, 3D7 (CQ-sensitive), NF54, and K1 (CQ-resistant) P. falciparum clones were obtained from MR4/American Type Culture Collection, Manassas, Va., USA under the accession numbers MR4-102 (3D7), MRA-1000 (NF54), and MR4-159 (K1). P. falciparum cultures were maintained in 25 cm² flasks. Parasites were cultured in RPMI 1640 (Sigma Aldrich, Austria) medium containing 10% human serum at a hematocrit of 5% (blood group 0 negative) at 37° C. under an atmosphere of 5% CO₂, 5% O₂ and 90% N₂. The medium was changed every 24 to 48 hours. The culture was diluted and fresh erythrocytes were added whenever the parasite density reached >1%.

### Determination of Parasitemia

A drop of blood (approx. 10 μL; sterile Pasteur pipette) was placed onto a clean slide and a thin smear was thus prepared. The smear was thoroughly dried and fixed in 100% MeOH solution. Giemsa stain (1:10) was prepared in water (10 mL) and dispensed over the fixed smear. The slide was stained for 15 minutes, after which it was thoroughly washed with distilled water. The slide was mounted under an oil immersion 100× objective and subsequently parasitemia was checked [(number of parasites/number of RBCs)×100=% parasitemia].

### In Vitro Assay

Stock solutions of compounds of formula I were prepared in DMSO (10 mg/mL). Stock concentrations of chloroquine and lumefantrine always remained 1 mg/mL and were prepared in 70% ethanol. Coating of culture plates with compounds of formula I and controls: 96 wells culture plates were coated as described previously (11). Working standards were prepared by diluting the stock solutions in RPMI1640 to obtain the desired final concentrations. Serial three-fold dilutions (seven concentrations and one drug-free control well) of the drugs (25 μL/well) were dispensed in duplicate into standard 96-well micro culture plates. Chloroquine was included as a reference drug in all experiments.

### Synchronization

After reaching a parasitemia of 5% or higher, 5 mL of cell medium mixture were centrifuged at 700 g for 5 minutes at room temperature (RT). Packed red cells were resuspended in 3 mL of 5% D-sorbitol (Sigma Aldrich, Austria) in water at RT and immediately centrifuged again at 700 g for 5 minutes at RT followed by three washes with 3 mL of RPMI1640 medium. riments.

### Addition of Parasites to the Drug/Compound Coated 96 Well Plates

Samples were diluted with RPMI1640 medium containing 0.5% Albumax II (GIBCO, Invitrogen, Vienna, Austria) by adding uninfected red blood cells to 1.5% hematocrit and 0.05% parasitemia. 150 μL of this cell medium mixture was then added to each well of 96-well plates precoated with test compounds and incubated at 37° C. for 72 hours in a gas mixture containing 5% CO₂, 5% O₂, and 90% N₂. After 72 hours the plates were frozen at −20° C. until the histidine rich protein II assay (23).

### HRP II Enzyme Linked Immunosorbant Assay (ELISA)

A highly sensitive HRPII ELISA was used based on two commercially available monoclonal antibodies (Immunology Consultants Laboratory, Inc., Newberg, Oreg.) directed against P. falciparum-specific HRPII: MPFM-55A, an immunoglobulin M antibody served as the primary capture antibody and HRP-conjugated MPFG-55P (Immunology Consultants Laboratory, Inc., Newberg, Oreg.) as secondary antibody. The ELISA was employed to assess growth inhibition as a measure of drug susceptibility as previously described. Optical density was measured at 450 nm using a standard ELISA plate reader (12-13).

### Determination of IC₅₀ Values

Hyperbolic concentration response curves were fitted to the data points by nonlinear least squares using the solver add-in of the Excel software.

Summary of In Vitro Antimalarial Potency (IC₅₀ [nM]) Assessed with Compounds of Formula I

Indicated in TABLE 2 are the IC₅₀ [nM] values for growth inhibition of the 3D7 (NF54) and K1 strains of P. falciparum for the compounds of formula I indicated with their formula numbers in TABLE 2. Unmarked IC₅₀ values for K1 and 3D7 strains of P. falciparum have been determined by HRP II enzyme linked immunosorbant assay (ELISA) as described above. Whereas IC₅₀ values marked with an asterisk “*” have been assessed for K1 as well as for NF54 strains of P. falciparum using [³H]-hypoxanthine as a marker for inhibition of parasite growth (14). Correlation of the formula numbers and chemical structures are set out in TABLE 1 of the example part.

TABLE 2

| Compound | I-1 | I-2 | I-3 | I-4 | I-5 | I-6 | I-7 | I-8 | I-9 | I-10 |
|---|---|---|---|---|---|---|---|---|---|---|
| 3D7/NF54* | 2460 | 680 | 51* | 38* | 59* | 121 | 198 | 140 | 264 | 96* |
| K1 | 507 | 435 | 11* | 8* | 12* | 29 | 99 | 13 | 29 | 26* |
| Compound | I-11 | I-12 | I-13 | I-16 | I-17 | I-18 | I-19 | I-20 | I-21 | I-22 |
| 3D7 | 186 | 611 | 457 | 131 | 1189 | 1567 | 153 | 1261 | 4122 | 368 |
| K1 | 45 | 92 | 168 | 54 | 209 | 349 | 40 | 100 | 613 | 251 |
| Compound | I-23 | I-24 | I-25 | I-26 | I-27 | I-28 | I-29 | I-30 | I-31 | I-32 |
| 3D7/NF54* | 226 | 134* | 74* | 472 | 177 | 309 | 97 | 57* | 101* | 148* |
| K1 | 34 | 36* | 18* | 8 | 50 | 90 | 5 | 18* | 30* | 52* |
| Compound | I-33 | I-34 | I-35 | I-36 | I-37 | I-38 | I-39 | I-40 | I-41 | I-42 |
| 3D7/NF54* | 27* | 20* | 14* | 670* | 63* | 24* | 9* | 6* | 70 | 2* |
| K1 | 8* | 5* | 4* | 182* | 16* | 8* | 3* | 2* | 12 | 0.5* |
| Compound | I-43 | I-44 | I-45 | I-46 | I-47 | I-48 | I-49 | I-50 | I-51 | I-52 |
| 3D7/NF54* | 119* | 1314 | 1863* | 26* | 16* | 14* | 9304 | 1746 | 2020 | 561 |
| K1 | 28* | 466 | 542* | 9* | 7* | 4* | 3228 | 403 | 134 | 52 |
| Compound | I-53 | I-54 | I-55 | I-56 | I-57 | I-58 | I-59 | I-60 | I-61 | I-62 |
| 3D7/NF54* | 197 | 276 | 6* | 2751* | 333* | 1693* | 1295 | 1362* | 18* | 704 |
| K1 | 29 | 85 | 2* | 827* | 76* | 467* | n.d. | 371* | 6* | 30 |
| Compound | I-63 | I-64 | I-65 | I-66 | I-67 | I-68 | I-69 | I-70 | I-71 | I-72 |
| 3D7/NF54* | 9488 | 1485 | 2554* | 2.4* | 8.8* | 8.6* | 5.5* | 1503* | 6.2* | 4.2* |
| K1 | 982 | 397 | 488* | 0.6* | 2.2* | 2.3* | 1.3* | 921* | 1.8* | 1.0* |
| Compound | I-73 | I-74 | I-75 | I-76 | I-77 | I-78 | I-79 | I-80 | I-81 | I-82 |
| 3D7/NF54* | 73* | 4.8* | 28* | n.d. | 256* | 205* | 258* | 47* | 36* | 24* |
| K1 | 21* | 1.2* | 7.1* | n.d. | 105* | 74* | 83* | 11* | 8.6* | 5.9* |
| Compound | I-83 | I-84 | I-85 | I-86 | I-87 | I-88 | I-89 | I-90 | I-91 | I-92 |
| 3D7/NF54* | 16* | 3.8* | 5.7* | n.d. | n.d. | n.d. | n.d. | n.d. | n.d. | n.d. |
| K1 | 3.5* | 1.0* | 1.2* | n.d. | n.d. | n.d. | n.d. | n.d. | n.d. | n.d. |

n.d.: not determined

In Vivo Assay

Primary biological assessment of in vivo antimalarial efficacy of the compounds of the present invention was assessed using the P. berghei rodent malaria 4-day suppressive test as recently described (14) (parasite: P. berghei, ANKA strain, GFP MRA-865; mice: NMRI mice, SPF, females, 25±2 g. free from Eperythrozoon coccoides and Haemobartonella muris). The experimental groups were treated with a single dose of compounds of the present invention (30 mg/kg) by the i.p. route. The compounds were prepared at an appropriate concentration, as a solution or suspension containing 7% Tween80/3% ethanol. Day 1 to 3 (24 h, 48 h and 72 h post-infection), the experimental groups of mice were treated again with the same dose and by the same route as on day 0. Day 4 (24 h after the last treatment, i.e. 96 h post-infection), blood smears from all animals are prepared and stained with Giemsa. Parasitemia was determined as described elsewhere (14). The difference between the mean value of the control group (taken as 100%) and those of the experimental groups was calculated and expressed as percent reduction (=activity).

Summary of in vivo antimalarial potency assessed with compounds of formula I.

Indicated in TABLE 3 below are the percent reduction values for growth inhibition of the Plasmodium berghei parasite for the compounds of formula I indicated with their formula numbers of TABLE 2. Correlation of the formula numbers and chemical structures are set out in TABLE 1 of the example part.

TABLE 3

| | | mg/kg | | | % of | |
|---|---|---|---|---|---|---|
| cage | compound | 4x | route | avg. Day 4 | control | % activity |
| 1 | I-40 | 30 | i.p. | 24.90 | 41.76 | 58.24 |
| 2 | I-55 | 30 | i.p. | 19.47 | 32.65 | 67.35 |
| 3 | I-39 | 30 | i.p. | 30.40 | 50.99 | 49.01 |
| 4 | I-42 | 30 | i.p. | 1.33 | 2.24 | 97.76 |

| | | |
|---|---|---|
| Co | Control Day 4 | 59.62 |

The compounds of the present invention show activity in the above ACTIVITY TESTING and are therefore indicated for the treatment of disorders (diseases) mediated, e.g. caused, by (infection of) protozoan organisms, such as apicomplexa, kinetoplastids, microsporidia or plasmodia. Infections mediated, e.g. caused, by protozoan organisms may result in disorders or diseases, such as malaria, Chagas disease, sleeping sickness, trichomoniasis, leishmaniasis, giardiasis, amebiasis, toxoplasmosis, trypanosomiasis (animal sleeping sickness), babesiosis, theileriosis, coccidiosis.

In another aspect the present invention provides
a compound of the present invention for use as a pharmaceutical,
the use of a compound of the present invention as a pharmaceutical,
   e.g. for the treatment of disorders mediated by protozoan organisms, such as apicomplexa, kinetoplastids, microsporidia, plasmodia.

For pharmaceutical use one or more compounds of the present invention may be used, e.g. one, or a combination of two or more compounds of the present invention, preferably one compound of the present invention is used.

A compound of the present invention may be used as a pharmaceutical in the form of a pharmaceutical composition.

In another aspect the present invention provides a pharmaceutical composition comprising a compound of the present invention in association with at least one pharmaceutically acceptable excipient, e.g. appropriate carrier and/or diluent, e.g. including fillers, binders, disintegrants, flow conditioners, lubricants, sugars or sweeteners, fragrances, preservatives, stabilizers, wetting agents and/or emulsifiers, solubilizers, salts for regulating osmotic pressure and/or buffers.

In another aspect the present invention provides
a pharmaceutical composition of the present invention for use of treating disorders which are mediated by protozoan organisms;
the use of a pharmaceutical composition of the present invention for treating disorders which are mediated by protozoan organisms.

In a further aspect the present invention provides a method of treating disorders which are mediated by protozoan organisms, e.g. including disorders as specified above, which treatment comprises administering to a subject in need of such treatment a therapeutically effective amount of an active compound of the present invention; e.g. in the form of a pharmaceutical composition.

Treatment of disorders (diseases) as used herein includes prophylaxis (prevention). For such treatment, the appropriate dosage will, of course, vary depending upon, for example, the chemical nature and the pharmacokinetic data of an active compound of the present invention used, the individual host, e.g. the body weight, the age and the individual condition of a subject in need of such treatment, the mode of administration and the nature and severity of the conditions being treated. However, in general, for satisfactory results in larger mammals, for example humans, an indicated daily dosage includes a range
from about 0.0001 g to about 1.5 g, such as 0.001 g to 1.5 g;
from about 0.001 mg/kg body weight to about 20 mg/kg body weight, such as 0.01 mg/kg body weight to 20 mg/kg body weight,
   for example administered in divided doses up to four times a day. Usually, children may receive half of the adult dose.

The compound of formula I-42 is a preferred compound of the present invention. It has, for example been determined that the IC₅₀ [nM] of a compound of formula I-42 against CQ sensitive NF54 is of 2 and against CQ resistant K1 is of 0.5.

It is indicated that for the treatment of diseases (disorders) mediated by protozoan organisms, such as malaria, an active compound of the present invention may be administered to larger mammals, for example humans, by similar modes of administration at similar dosages than conventionally used with approved antimalarials, e.g. in combination with an artimisinin derivative, e.g. Artemether®, or in combination with a drug having a similar mode of action and pharmacokinetics as artemisinin derivatives.

An active compound of the present invention may be administered by any conventional route, for example enterally, e.g. including nasal, buccal, rectal, oral administration; parenterally, e.g. including intravenous, intraarterial, intramuscular, intracardiac, subcutaneous, transdermal (diffusion through the intact skin), transmucosal (diffusion through a mucous membrane), inhalational administration; e.g. in form of coated or uncoated tablets, capsules, (injectable) solutions, solid solutions, suspensions, dispersions, solid dispersions; e.g. in the form of ampoules, vials, in the form of inhaler powder, drops, sprays, or in the form of suppositories.

An active compound of the present invention may be administered in the form of a pharmaceutically acceptable salt, or in free form; optionally in the form of a solvate. A compound of the present invention in the form of a salt and/or in the form of a solvate exhibits the same order of activity as a compound of the present invention in free form.

An active compound of the present invention may be used for any method or use as described herein alone or in combination with one or more, at least one, other drug substance.

In another aspect the present invention provides
A combination of a compound of the present invention with at least one second drug substance;
A pharmaceutical combination comprising a compound of the present invention in combination with at least one second drug substance;
A pharmaceutical composition comprising a compound of the present invention in combination with at least one second drug substance and one or more pharmaceutically acceptable excipient(s);
A compound of the present invention in combination with at least one second drug substance, e.g. in the form of a pharmaceutical combination or composition, for use in any method as defined herein, e.g.
A combination, a pharmaceutical combination or a pharmaceutical composition, comprising a compound of the present invention and at least one second drug substance for use as a pharmaceutical;
The use as a pharmaceutical of a compound of the present invention in combination with at least one second drug substance, e.g. in the form of a pharmaceutical combination or composition;
The use of a compound of the present invention for the manufacture of a medicament for use in combination with a second drug substance;
A method for treating disorders mediated by protozoan organisms in a subject in need thereof, comprising co-administering, concomitantly or in sequence, a therapeutically effective amount of a compound of the present invention and at least one second drug substance, e.g. in the form of a pharmaceutical combination or composition;
A compound of the present invention in combination with at least one second drug substance, e.g. in the form of a pharmaceutical combination or composition, for use in the preparation of a medicament for use in disorders mediated by protozoan organisms.

Combinations include fixed combinations, in which a compound of the present invention and at least one second drug substance are in the same formulation; kits, in which a compound of the present invention and at least one second drug substance in separate formulations are provided in the same package, e.g. with instruction for co-administration; and free combinations in which a compound of the present invention and at least one second drug substance are packaged separately, but instruction for concomitant or sequential administration are given.

In another aspect the present invention provides
A pharmaceutical package comprising a first drug substance which is a compound of the present invention and at least one second drug substance, beside instructions for combined administration;
A pharmaceutical package comprising a compound of the present invention beside instructions for combined administration with at least one second drug substance;
A pharmaceutical package comprising at least one second drug substance beside instructions for combined administration with a compound of the present invention.

Treatment with combinations according to the present invention may provide improvements compared with single treatment.

In another aspect the present invention provides
A pharmaceutical combination comprising an amount of a compound of the present invention and an amount of a second drug substance, wherein the amounts are appropriate to produce a synergistic therapeutic effect;
A method for improving the therapeutic utility of a compound of the present invention comprising co-administering, e.g. concomitantly or in sequence, of a therapeutically effective amount of a compound of the present invention and a second drug substance.
A method for improving the therapeutic utility of a second drug substance comprising co-administering, e.g. concomitantly or in sequence, of a therapeutically effective amount of a compound of the present invention and a second drug substance.

A combination of the present invention and a second drug substance as a combination partner may be administered by any conventional route, for example as set out above for a compound of the present invention. A second drug may be administered in dosages as appropriate, e.g. in dosage ranges which are similar to those used for single treatment, or, e.g. in case of synergy, even below conventional dosage ranges.

Pharmaceutical compositions according to the present invention may be manufactured according, e.g. analogously, to a method as conventional, e.g. by mixing, granulating, coating, dissolving or lyophilizing processes. Unit dosage forms may contain, for example, from about 0.1 mg to about 1500 mg, such as 1 mg to about 1000 mg.

By the term “second drug substance” is meant a chemotherapeutic drug, especially any chemotherapeutic agent other than an active compound of the present invention, such as a compound of formula I.

For example, a second drug substance as used herein includes drugs or drug combinations useful for the treatment of diseases mediated by protozoan organisms, e.g. antimalarials.

The identification of the molecular target(s) of arylamino alcohol antimalarials would significantly contribute to a deeper understanding of resistance mechanisms against this compound class. Furthermore, knowledge about the target(s) of a whole compound class would greatly assist the discovery of novel antimalarial drugs.

Certain compounds of the present invention fulfill all requirements to serve as starting points for the introduction of target identification tools at tolerant positions of the scaffold while keeping high antimalarial potency. Such tools are photoactive functional groups in combination with chemical baits. The former ones establish a covalent bond between the drug molecule and molecular target(s) upon irradiation with light, the latter ones allow ligand-directed capture, enrichment and subsequent purification of crosslinking products prior to concluding analysis.

Photoactive groups can be incorporated into molecules of formula I of the present invention by use of amine building blocks bearing such photoactive functionalities. The synthesis protocols for such tool compounds of formula I remain unchanged with respect to the general methods provided herewith.

Salicylamide based aminoalcohols of formula I of the present invention may incorporate photoactive groups either in the amide- or in the amine part of the molecule. Surprisingly, both positions in a compound of formula I of the present invention were found to be tolerant for such modifications, whereby the antimalarial activity is retained, e.g. exemplified with compounds I-43 and I-44 of the present invention (TABLE 1), respectively. The antimalarial potency of arylamino alcohols bearing photoaffinity labels (IC₅₀ against strains K1 and 3D7/NF54; values marked with asterisk: K1/NF54, [³H]-hypoxanthine incorporation assay; values without label: K1/3D7, HRP2-based ELISA) is set out in TABLE 4 below:

TABLE 4

| | | |
|---|---|---|
| K1 [IC₅₀] | 28* | 466 |
| 3D7/NF54* | 119* | 1314 |
| [IC₅₀] | | |
| | 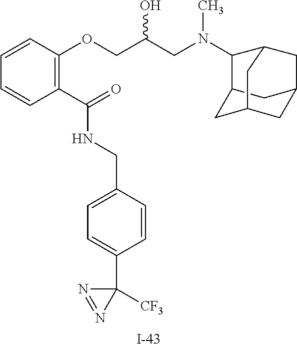 | 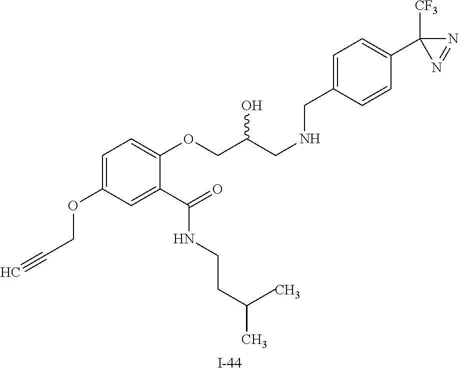 |

Photoaffinity labels may be derived e.g. from groups of benzophenones or diazirines (e.g. compounds I-49 and I-43 of the present invention), provided that the reagents for introduction of such photophors bear an appropriate amine functionality. Diazirines have been selected as the preferred photoactive group due to high cross-linking efficiency of carbenes.

Furthermore, biologically active salicylamides already equipped with a photoactivatable group (e.g. compound I-43 of the present invention) tolerate further incorporation of an alkyne moiety as bait, and retain antimalarial activity (e.g. compound I-44 of the present invention).

The cross-linking product of such a drug to the unknown target (TARGET in REACTION SCHEME 3 below) can be captured by a prey-tool. As depicted in REACTION SCHEME 3, the prey-tool offers an azide functionality for copper catalyzed cycloaddition onto the alkyne still present in the cross-linking product (REACTION SCHEME 3). The copper catalyzed cycloaddition of alkynes and azides (CuAAC) is well known (15). CuAAC can be performed in water under physiological conditions.

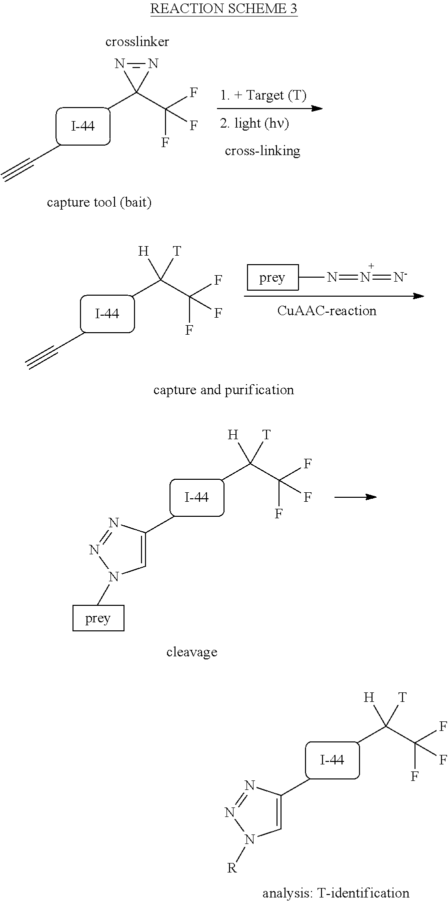

In REACTION SCHEME 3 above the expression “I-44” indicates a compound of formula I-44 of the present invention. T indicates the target.

In another aspect the present invention provides the use of a compound of the present invention, e.g. of formula I, which compound comprises a photoaffinity label, e.g. a diazirinyl group, or a benzophenone group, for the identification of the molecular target(s) of arylamino alcohol containing drugs, e.g. antimalarial compounds.

In a further aspect the present invention provides the use of a compound of formula

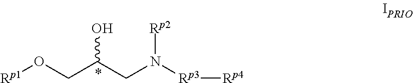

wherein
R^{p1} is a group of formula

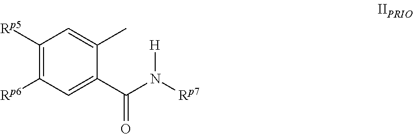

R^{p2} is H, (C₁₋₈)alkyl, or (C₃₋₆)cycloalkyl, wherein alkyl or cycloalkyl optionally are substituted by
(C₁₋₄)alkyl,
(C₁₋₄)alkoxy, or
phenyl, which phenyl optionally is substituted one or morefold by (C₁₋₆)alkoxy, R^{p3} is not present or is (C₁₋₈)alkylene, which alkylene is unsubstituted, or substituted by (C₁₋₈)alkyl; or
   R^{p2} and R^{p3} together with the nitrogen atom to which they are attached form a heterocyclic ring, optionally comprising a further heteroatom,

### R^{p4} is

H, e.g. if R³ is present,
(C₅₋₁₂)cycloalkyl, which cycloalkyl optionally is substituted by (C₁₋₄)alkyl;
(C₁₋₄)alkyl, optionally substituted by phenyl,
if R^{p3} is present, (C₆₋₁₂)aryl, which aryl optionally is substituted by (C₁₋₆)alkyl, (C₂₋₆)alkenyl, (C₂₋₆)alkynyl, (C₁₋₆)alkyloxy, (C₂₋₆)alkenyl-(C₁₋₄)alkylenoxy, HC≡C—(C₁₋₆)alkylenoxy, halogen, halogenated (C₁₋₄)alkyl, phenylcarbonyl, or diazirinyl of formula

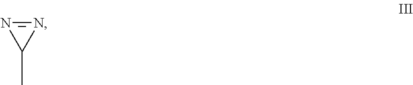

R^{p5} and R^{p6} independently of each other are H, halogen, (C₁₋₄)alkyl, (C₂₋₄)alkenyl, (C₂₋₄)alkynyl, (C₁₋₄)alkoxy, (C₂₋₆)alkenyl-(C₁₋₄)alkylenoxy, HC≡C—(C₁₋₆)alkylenoxy, and

### R^{p7} is

(C₁₋₈)alkyl or (C₆₋₁₂)aryl, wherein alkyl or aryl is unsubstituted or substituted, in particular aryl is substituted, wherein substituted alkyl or aryl are substituted by
halogen,
(C₁₋₄)alkyl, (C₂₋₄)alkenyl, (C₂₋₄)alkynyl,
halogenated (C₁₋₄)alkyl,
(C₁₋₄)alkoxy, (C₂₋₆)alkenyl-(C₁₋₄)alkylenoxy, HC≡C—(C₁₋₆)alkylenoxy, or
(C₆₋₁₂)aryl, which aryl optionally is substituted by halogen, (C₁₋₄)alkyl, (C₂₋₄)alkenyl, (C₂₋₄)alkynyl, halogenated (C₁₋₄)alkyl, e.g. CF₃, (C₁₋₄)alkoxy, (C₂₋₆)alkenyl-(C₁₋₄)alkylenoxy, HC≡C—(C₁₋₆)alkylenoxy, phenylcarbonyl, or diazirinyl of formula III,
   for use in the treatment of disorders mediated by protozoan organisms;
   and, in a further aspect,

A compound of formula I_{PRIO}, as defined above, wherein R^{p4} is (C₈₋₁₂)cycloalkyl and the other residues are as defined above, and the compounds selected from
2-{2-Hydroxy-3-[4-(3-trifluoromethyl-3H-diazirin-3-yl)-benzylamino]-propoxy}-N-(3-methyl-butyl)-5-prop-2-ynyloxy-benzamide (I-44),
N-(4-Fluoro-phenyl)-2-{2-hydroxy-3-[4-(3-trifluoromethyl-3H-diazirin-3-yl)-benzylamino]-propoxy}-5-prop-2-ynyloxy-benzamide (I-45),
N-(4-Fluoro-phenyl)-2-[2-hydroxy-3-(4-phenyl-piperidin-1-yl)-propoxy]-benzamide (I-46),
2-[3-(4-Benzyl-piperidin-1-yl)-2-hydroxy-propoxy]-N-(4-fluoro-phenyl)-benzamide (I-47),
2-[3-(4-Benzhydryl-piperazin-1-yl)-2-hydroxy-propoxy]-N-(4-fluoro-phenyl)-benzamide (I-48),
2-[3-(4-Benzoyl-benzylamino)-2-hydroxy-propoxy]-N-benzyl-benzamide (I-49),
N-Benzyl-2-[2-hydroxy-3-(3-phenyl-propylamino)-propoxy]-benzamide (I-50),
2-{3-[4-(2,3-Dimethyl-phenyl)-piperazin-1-yl]-2-hydroxy-propoxy}-N-(4-methoxy-benzyl)-benzamide (I-51),
2-(2-Hydroxy-3-propylamino-propoxy)-N-p-tolyl-benzamide (I-54),
2-(3-Butylamino-2-hydroxy-propoxy)-N-(3-methyl-butyl)-benzamide (I-56),
2-(3-Dibutylamino-2-hydroxy-propoxy)-N-(3-methyl-butyl)-benzamide (I-57),
2-[2-Hydroxy-3-(3-methyl-butylamino)-propoxy]-N-(3-methyl-butyl)-benzamide (I-58),
2-(3-Benzylamino-2-hydroxy-propoxy)-N-(3-methyl-butyl)-benzamide (I-59)
2-[2-Hydroxy-3-(4-trifluoromethyl-benzylamino)-propoxy]-N-(3-methyl-butyl)-benzamide (I-60),
2-(3-Dibutylamino-2-hydroxy-propoxy)-N-(4-fluoro-phenyl)-benzamide (I-61), and
N-Benzyl-2-{2-hydroxy-3-[(4-methoxy-3-prop-2-ynyloxy-benzyl)-propyl-amino]-propoxy}-benzamide (I-63),
   e.g. the compound of formula I-1 to I-13 and I-16 to I-64, optionally in the form of a salt.

In the following examples all temperatures are in degree Celsius (° C.) and are uncorrected.

NMR: If not otherwise indicated, all ¹H/¹³C-spectra were measured in CDCl₃ at 23° C. on a Brucker Avance 400 NMR-spectrometer. Chemical shifts (δ) are calibrated to residual signal of the solvent used. Analysis was performed with free software SpinWorks version 3.1.7. (Copyright © 2010, Kirk Marat University of Manitoba).

MS: MS-spectra were recorded on a AB Sciex QStar Elite and were processed with Analyst QS software.

The following abbreviations are used
br broad
calc. calculated,
d day(s)
DIPEA N,N-diisopropyl-ethylamine
DMF N,N-dimethylformamide
DMSO dimethylsulfoxide
dyn dynamic
EDC-HCl 1-ethyl-3-(3-dimethylaminopropyl)-carbodiimide in the form of a HCl salt
EtOAc ethyl acetate
EtOH ethanol
h hour(s)
HMBC Heteronuclear Multiple Bond Correlation
HOBt 1-hydroxy-benzotriazole
MeOH methanol
min minute(s)
MTBE tert-butyl-methylether
obs. observed
PE petrol ether
RT room temperature

### Example 1

### 2-{3-[(Adamantan-1-ylmethyl)-amino]-2-hydroxy-propoxy}-N-p-tolyl-benzamide (compound of formula I-40)

The compound was prepared according to REACTION SCHEME 1 above.

### Step a1) 2-Hydroxy-N-p-tolyl-benzamide

One drop of boron trifluoride diethyl etherate was added to a solution of 2-hydroxybenzoic acid phenyl ester (1 g, 46.7 mmol) and 4-toluidine (0.5 g, 46.7 mmol) in toluene (5 mL). The reaction mixture obtained was kept at RT overnight. A crystalline precipitate formed, the crystals were collected by filtration, washed 3 times with cold MTBE and dried in vacuo to afford 2-hydroxy-N-p-tolyl-benzamide in the form of colorless crystals (0.81 g, 76% of theory). Chemical characterization data correspond to data derived from the known compound (CAS 7164-80-9).

### Step b1) 2-Oxiranylmethoxy-N-p-tolyl-benzamide

Freshly powdered KOH (0.7 g, 13.2 mmol) was added to a solution of 2-hydroxy-N-p-tolyl-benzamide (3 g, 13.2 mmol) in MeOH (20 mL) and the mixture was kept at 60° C. on a rotary evaporator. A homogeneous solution formed from which the solvent was removed under reduced pressure. Racemic epichlorohydrin (10 mL) was added and the mixture obtained was heated to reflux for 5 min. The excess of epichlorohydrin was removed under reduced pressure and the crude oil obtained was diluted with EtOAc (50 mL), washed three times with brine, dried over Na₂SO₄ and concentrated under reduced pressure to yield a grey solid which was recrystallized from EtOAc to afford 2-oxiranylmethoxy-N-p-tolyl-benzamide in the form of colorless crystals (1.6 g, 43% of theory). Chemical characterization data correspond to data derived from the known compound (CAS 81500-04-01).

### Step c1) 2-{3-[(Adamantan-1-ylmethyl)-amino]-2-hydroxy-propoxy}-N-p-tolyl-benzamide

A mixture of 2-oxiranylmethoxy-N-p-tolyl-benzamide (0.1 g, 0.353 mmol) and C-adamantan-1-yl-methylamine (0.058 g, 0.353 mmol) in EtOH (4 mL) was prepared in a screw cap tube. The tube was sealed and kept in an oil bath at 135° C. for 7 h and the reaction mixture was cooled to RT. From the mixture obtained the solvent was removed under reduced pressure on a rotary evaporator, the resulting oily residue was purified by chromatography (silica gel, EtOAc) and 2-{3-[(adamantan-1-ylmethyl)-amino]-2-hydroxy-propoxy}-N-p-tolyl-benzamide (81 mg, 52% of theory) was obtained. Chemical characterization data are set out in TABLE 1 below (compound of formula I-40).

### Example 2

### 2-{2-Hydroxy-3-[4-(3-trifluoromethyl-3H-diazirin-3-yl)-benzylamino]propoxy}-N-(3-methyl-butyl)-5-prop-2-ynyloxy-benzamide (compound of formula I-44)

The compound was prepared according to REACTION SCHEME 4 below:

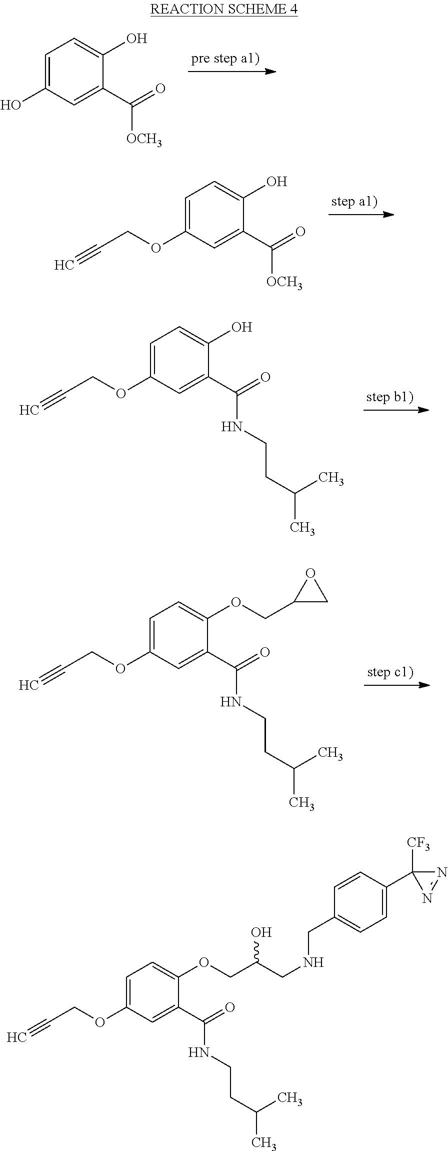

### Pre step a1) 2-Hydroxy-5-prop-2-ynyloxy-benzoic acid methyl ester

Propargylbromide (30.7 mL, 285 mmol) was added to a suspension of 2,5-dihydroxy-benzoic acid methyl ester (40 g, 237.9 mmol) and K₂CO₃ (40 g, 285 mmol) in acetone (250 mL) and the reaction mixture was kept at reflux for 20 h. The heterogeneous mixture obtained was filtered and the volatile materials were removed under reduced pressure. The residual oil was neutralized with 2N HCl. The mixture obtained was extracted with EtOAc, the organic phase was washed once with saturated NaHCO₃-solution and dried over MgSO₄. From the dried solution the solvent was removed under reduced pressure and the oily residue obtained was cooled in an ice-bath for 30 min Upon trituration in MTBE with a glass rod crystallization occurred and the crystals were collected and dried to afford 2-hydroxy-5-prop-2-ynyloxy-benzoic acid methyl ester afford (39 g, 80% of theory). Chemical characterization data correspond to data derived from the known compound (CAS 73922-94-8).

### Step a1) 2-Hydroxy-N-(3-methyl-butyl)-5-prop-2-ynyloxy-benzamide

A solution of 2-hydroxy-5-prop-2-ynyloxy-benzoic acid methyl ester (1.36 g, 6.6 mmol) and isoamylamine (1.15 g, 13.2 mmol) in toluene (5 mL) was kept at RT for 2 d. The mixture obtained was diluted with EtOAc (50 mL), washed three times with 2N HCl, dried over Na₂SO₄ and concentrated under reduced pressure. The resulting oil crystallized upon trituration with MTBE. The crystals were collected and washed once with cold MTBE to yield 2-hydroxy-N-(3-methyl-butyl)-5-prop-2-ynyloxy-benzamide (1.3 g, 75% of theory). ¹H NMR: δ=0.96 (6H), 1.48-1.55 (2H), 1.63-1.73 (1H), 2.52 (1H), 3.42-3.50 (2H), 4.64 (2H), 6.17 (1H), 6.93 (1H), 6.93 (1H), 7.08 (1H), 11.88 (1H); ¹³C NMR: δ=22.58, 26.10, 38.24, 38.42, 57.29, 75.97, 78.66, 112.24, 114.51, 119.44, 122.03, 149.68, 156.53, 169.58.

### Step b1) N-(3-Methyl-butyl)-2-oxiranylmethoxy-5-prop-2-ynyloxy-benzamide

To a solution of 2-hydroxy-N-(3-methyl-butyl)-5-prop-2-ynyloxy-benzamide (0.2 g, 0.765 mmol) in MeOH (20 mL) was added freshly powdered KOH (0.47 g, 0.819 mmol). The mixture obtained was kept at 60° C. on a rotary evaporator. A homogeneous solution formed from which the solvent was removed under reduced pressure. Racemic epichlorohydrin (5 mL) was added and the mixture was heated to reflux for 5 min From the mixture obtained the excess of epichlorohydrin was removed under reduced pressure, the crude oil obtained was diluted with EtOAc (50 mL), the dilution obtained was washed three times with brine, dried over Na₂SO₄, concentrated under reduced pressure to yield a grey solid. The solid obtained was recrystallized from EtOAc to afford N-(3-methyl-butyl)-2-oxiranylmethoxy-5-prop-2-ynyloxy-benzamide in the form of colorless crystals (0.19 g, 78% of theory). ¹H NMR: δ=0.93 (6H), 1.46-1.54 (2H), 1.63-1.73 (1H), 2.49 (1H), 2.75-2.77 (1H), 2.90-2.92 (1H), 3.18-3.23 (1H), 3.42-3.48 (2H), 3.99 (1H), 4.36 (1H), 4.66 (2H), 6.87 (1H), 7.00 (1H), 7.79 (1H), 7.92 (1H); ¹³C NMR: δ=22.54, 26.02, 38.29, 38.34, 44.45, 49.81, 56.55, 70.03, 75.72, 78.49, 114.83, 117.30, 120.09, 123.40, 151.12, 152.43, 164.52.

### Step c1) 2-{2-Hydroxy-3-[4-(3-trifluoromethyl-3H-diazirin-3-yl)-benzylamino]-propoxy}-N-(3-methyl-butyl)-5-prop-2-ynyloxy-benzamide

A solution of N-(3-methyl-butyl)-2-oxiranylmethoxy-5-prop-2-ynyloxy-benzamide (0,123 g, 0.387 mmol) and 4-(3-trifluoromethyl-3H-diazirin-3-yl)-benzylamine (0.084 g, 0.387 mmol) in EtOH (4 mL) was kept in a sealed tube at 135° C. for 4 h. The mixture obtained was cooled to RT and the solvent was removed at reduced pressure. The oily residue obtained was purified by chromatography on silica gel (EtOAc) to afford 2-{2-hydroxy-3-[4-(3-trifluoromethyl-3H-diazirin-3-yl)-benzylamino]-propoxy}-N-(3-methyl-butyl)-5-prop-2-ynyloxy-benzamide in the form of a yellowish oil (88 mg, 43% of theory). Chemical characterization data is set out in TABLE I below (compound of formula I-44).

### Example 3

### 2-[3-(Adamantan-2-yl-methyl-amino)-2-hydroxy-propoxy]-N-[4-(3-trifluoromethyl-3H-diazirin-3-yl)-benzyl]-benzamide (compound of formula I-43)

The compound was prepared according to REACTION SCHEME 2 above.

### Step a2) 2-Oxiranylmethoxy-benzoic acid methyl ester

Freshly powdered KOH (0.74 g, 13.1 mmol) was added to a solution of 2-hydroxybenzoic acid methyl ester (2 g, 13.1 mmol) in MeOH (15 mL) and the mixture was kept at 60° C. on a rotary evaporator. A homogeneous solution formed from which the solvent was removed under reduced pressure. Racemic epichlorohydrin (10 mL) was added and the mixture obtained was heated to reflux for 5 min. The excess of epichlorohydrin was removed under reduced pressure and the resulting oil was diluted with EtOAc (30 mL). The dilution obtained was washed three times with brine, dried over Na₂SO₄, concentrated under reduced pressure and purified by chromatography (MTBE/PE 3:2) to yield 2-oxiranylmethoxy-benzoic acid methyl ester (1.48 g, 54% of theory). Chemical characterization data correspond to data derived from the known compound (CAS 22589-46-4).

### Step b2) 2-[3-(Adamantan-2-yl-methylamino)-2-hydroxypropoxy]-benzoic acid methyl ester

A mixture of 2-oxiranylmethoxy-benzoic acid methyl ester (2.57 g, 12.34 mmol) and adamantan-2-yl-methylamine (2.04 g, 12.3 mmol) in EtOH (4 mL) was prepared in a screw cap tube. The tube was sealed and kept in an oil bath at 90° C. for 16 h. The mixture obtained was cooled to RT, the solvent was removed under reduced pressure on a rotary evaporator and the oily residue obtained was purified by chromatography on silica gel (EtOAc) to yield 2-[3-(adamantan-2-yl-methylamino)-2-hydroxypropoxy]-benzoic acid methyl ester in the form of a yellowish oil (4.06 g, 88% of theory). Chemical characterization data is set out in TABLE I below (compound of formula INT-14).

### Step c2) 2-[3-(Adamantan-2-yl-methyl-amino)-2-hydroxy-propoxy]-benzoic acid

2-[3-(Adamantan-2-yl-methylamino)-2-hydroxypropoxy]-benzoic acid methyl ester (3 g, 13.2 mmol) was saponified with a solution of KOH (4.47 g, 79.53 mmol) in MeOH (50 mL) over 24 h at RT. From the mixture obtained the solvent was removed under reduced pressure, the residue was neutralized with 2N HCl, the mixture obtained was heated in water (50 mL) and cooled to RT. Crystallization occurred and the crystals were collected by filtration, washed three times with warm water and dried in vacuo to afford 2-[3-(adamantan-2-yl-methyl-amino)-2-hydroxy-propoxy]-benzoic acid in the form of white crystals (2.9 g, 90% of theory). Chemical characterization data are set out in TABLE I below (compound of formula INT-15).

### Step d2) 2-[3-(Adamantan-2-yl-methyl-amino)-2-hydroxy-propoxy]-N-[4-(3-trifluoromethyl-3H-diazirin-3-yl)-benzyl]-benzamide

A mixture of 2-[3-(adamantan-2-yl-methyl-amino)-2-hydroxy-propoxy]-benzoic acid (1.0 g, 2.78 mmol), EDC-HCl (0.59 g, 3.07 mmol), HOBt (0.42 g, 3.07 mmol) and DIPEA (0.76 g, 5.84 mmol) in DMF (5 mL) was stirred at RT for 30 min. 4-(3-Trifluoromethyl-3H-diazirin-3-yl)-benzylamine (0.60 g, 2.79 mmol) was added and the mixture was stirred for an additional 2 h. The mixture obtained was diluted with EtOAc (50 mL), the dilution obtained was washed three times with water and dried over Na₂SO₄. From the solution obtained the solvent was removed under reduced pressure and the oily residue was purified by chromatography on silica gel (EtOAc) to afford 2-[3-(adamantan-2-yl-methyl-amino)-2-hydroxy-propoxy]-N-[4-(3-trifluoromethyl-3H-diazirin-3-yl)-benzyl]-benzamide in the form of a yellowish oil (1.04 g, 67% of theory) which was stored in the dark at +4° C. Chemical characterization data are set out in TABLE 1 below (compound of formula I-43).

Analogously to the methods as described in Examples 1 to 3, but using appropriate starting materials (intermediates) compounds of formula I as indicated in TABLE 1 below having CHARACTERIZATION DATA as defined in TABLE 1 below, were obtained.

TABLE 1

| | |
|---|---|
| 2-[3-(Adamantan-1-ylamino)-2-hydroxy-propoxy]-N- benzyl-benzamide (I-1) ¹³C NMR: δ = 29.55, 36.63, 42.33, 42.90, 44.03, 50.54, 68.31, 71.22, 113.01, 121.72, 122.28, 127.28, 128.01, 128.64, 132.34, 132.71, 139.03, 157.05, 165.40; MS: [M + H]⁺ calc. 435.2642, obs. 435.2546 | 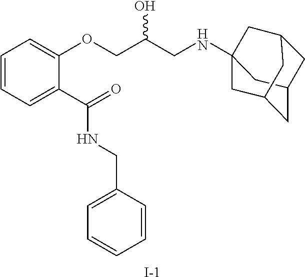 [SMILES: O=C(NCc1ccccc1)c1ccccc1OCC(O)CNC12CC3CC(CC(C3)C1)C2 ; [I-]] |
| 2-[3-(Adamantan-1-ylamino)-2-hydroxy-propoxy]-N- (2-methoxy-benzyl)-benzamide (I-2) ¹³C NMR: δ = 29.60, 36.68, 39.30, 42.41, 42.88, 50.77, 55.69, 68.39, 71.39, 110.58, 112.98, 120.83, 121.71, 122.71, 127.05, 128.67, 129.60, 132.29, 132.59, 156.99, 157.66, 165.44; MS: [M + H]⁺ calc. 465.27480, obs. 465.2548 | 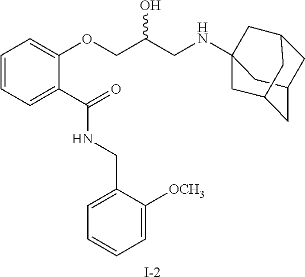 |
| 2-[3-(Adamantan-1-ylamino)-2-hydroxy-propoxy]-N- (4-fluoro-phenyl)-benzamide (I-3) ¹H NMR: δ = 1.46-1.59 (9H), 1.60-1.68 (3H), 2.02 (3H), 2.65 (1H), 2.85 (1H), 3.91-3.99 (1H), 4.02 (1H), 4.23 (1H), 6.93 (1H), 6.96-7.03 (2H), 7.08 (1H), 7.42 (1H), 7.73-7.80 (2H), 8.21 (1H), 10.16 (1H); ¹³C NMR: δ = 29.56, 36.62, 42.24, 43.02, 50.61, 68.44, 71.42, 112.68, 115.39, 121.85, 122.29, 122.35 (HMBC), 132.48, 133.16, 135.04, 156.69, 159.25, 163.52 | 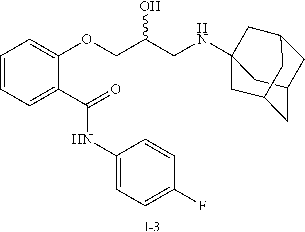 |
| 2-[3-(3,5-Dimethyl-adamantan-1-ylamino)-2-hydroxy- propoxy]-N-(4-fluoro-phenyl)-benzamide (I-4) ¹H NMR: δ = 0.79 (6H), 1.01-1.29 (10H), 1.33-1.48 (2H), 2.07-2.12 (1H), 2.70 (1H), 2.90 (1H), 3.97-4.08 (2H), 4.26 (1H), 6.95 (1H), 6.98-7.05 (2H), 7.12 (1H), 7.45 (1H), 7.74-7.80 (2H), 8.23 (1H), 10.11 (1H); ¹³C NMR: δ = 30.26, 30.29, 32.54, 41.41, 42.61, 42.90, 49.03, 50.85, 52.96, 68.20, 71.32, 112.66, 115.52, 122.00, 122.27, 122.50, 132.60, 133.19, 135.08, 156.67, 159.31, 163.52 | 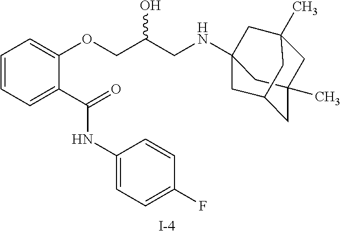 |
| 2-[3-(4-Adamantan-1-yl-piperazin-1-yl)-2-hydroxy- propoxy]-N-(4-fluoro-phenyl)-benzamide (I-5) ¹H NMR: δ = 1.56-1.74 (12H), 2.10 (3H), 2.35-2.44 (2H), 2.44 (1H), 2.58-2.76 (7H), 4.03 (1H), 4.15-4.22 (1H), 4.32 (1H), 6.96 (1H), 6.99-7.06 (2H), 7.12 (1H), 7.46 (1H), 7.72-7.81 (2H), 8.25 (1H), 10.13 (1H); ¹³C NMR: δ = 29.80, 37.01, 38.61, 38.79, 44.26, 54.26, 59.76, 65.07, 71.01, 112.72, 115.50, 122.05, 122.13, 122.48, 132.68, 133.21, 135.14, 156.62, 159.28, 163.45 | 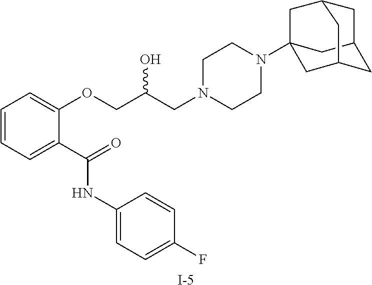 |
| 2-[3-(Adamantan-2-ylamino)-2-hydroxy-propoxy]-N- benzyl-benzamide (I-6) ¹³C NMR: δ = 27.55, 27.66, 31.33 (2x), 31.69, 32.70, 37.52, 37.57, 37.84, 44.11, 48.99, 62.01, 67.79, 71.06, 113.01, 121.82, 122.40, 127.33, 128.19, 128.65, 132.40, 132.73, 139.08, 157.02, 165.37; MS: [M + H]⁺ calc. 435.26420, obs. 435.2460 | 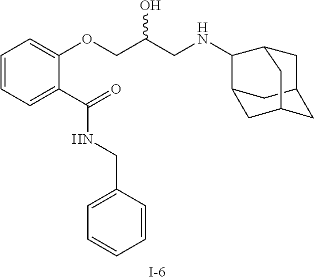 |
| 2-[3-(Adamantan-2-ylamino)-2-hydroxy-propoxy]-N- (2-methoxy-benzyl)-benzamide (I-7) ¹³C NMR: δ = 26.69, 26.95, 29.13, 29.68, 30.50, 30.55, 36.85, 36.90, 37.10, 39.39, 50.49, 55.73, 64.44, 65.35, 71.21, 110.65, 113.56, 120.68, 122.16, 123.45, 126.87, 128.64, 129.10, 131.85, 132.69, 156.54, 157.67, 165.80; MS: [M + H]⁺ calc. 465.27480, obs. 465.2602 | 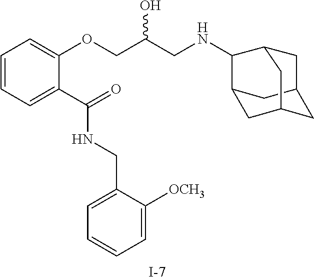 |
| 2-[3-(Adamantan-2-ylamino)-2-hydroxy-propoxy]-N- (4-methoxy-benzyl)-benzamide (I-8) ¹³C NMR: δ = 27.63, 27.72, 31.38, 31.90, 32.91, 37.58, 37.62, 37.90, 43.54, 48.88, 55.35, 61.88, 68.01, 71.04, 112.95, 114.00, 121.75, 122.41, 129.45, 131.13, 132.37, 132.67, 157.03, 158.96, 165.25; MS: [M + H]⁺ calc. 465.27480, obs. 465.2538 | 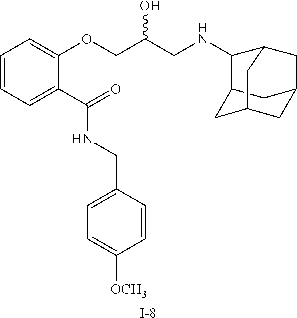 |
| 2-[3-(Adamantan-2-ylamino)-2-hydroxy-propoxy]-N- phenethyl-benzamide (I-9) ¹³C NMR: δ = 27.65, 27.75, 31.46 (2x), 32.04, 32.93, 35.86, 37.61, 37.66, 37.92, 41.28, 48.97, 62.05, 68.21, 71.49, 113.18, 121.78, 122.57, 126.34, 128.58, 129.01, 132.27, 132.58, 139.83, 157.03, 165.47; MS: [M + H]⁺ calc. 449.27990, obs. 449.2650 | 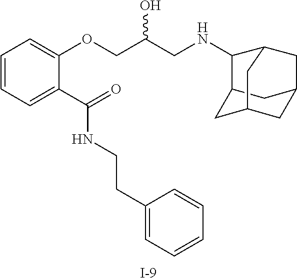 |
| 2-[3-(Adamantan-2-ylamino)-2-hydroxy-propoxy]-5- chloro-N-phenethyl-benzamide (I-10) ¹³C NMR: δ = 27.63, 27.72, 31.43, 31.45, 32.01, 32.91, 35.72, 37.58, 37.64, 37.89, 41.35, 48.86, 62.04, 68.12, 71.97, 114.76, 123.99, 126.38, 127.12, 128.58, 128.96, 131.94, 132.16, 139.62, 155.56, 164.14; [M + H]⁺ calc. 483.2410, obs. 483.2317 | 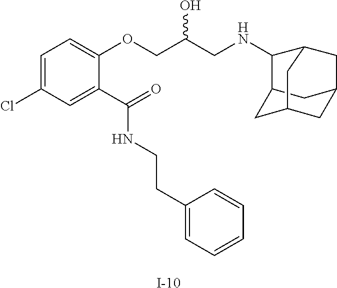 |
| 2-[3-(Adamantan-2-ylamino)-2-hydroxy-propoxy]-N- (3-phenyl-propyl)-benzamide (I-11) ¹³C NMR: δ = 27.44, 27.56, 31.23 (2x), 31.31, 31.43, 32.41, 33.55, 37.43, 37.49, 37.72, 39.75, 49.25, 62.43, 67.70, 71.30, 113.02, 121.79, 122.68, 125.92, 128.45, 128.50, 132.23, 132.52, 141.93, 156.85, 165.51; MS: [M + H]⁺ calc. 463.2955, obs. 463.2872 | 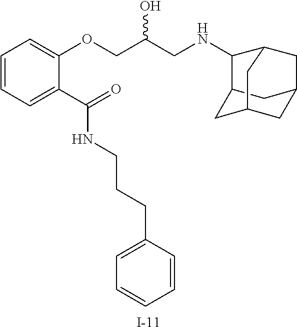 |
| 2-[3-(Adamantan-2-ylamino)-2-hydroxy-propoxy]-N- propyl-benzamide (I-12) ¹³C NMR: δ = 11.79, 22.98, 27.66, 27.75, 31.45, 31.49, 32.12, 32.96, 37.65, 37.68, 37.92, 41.83, 49.16, 62.21, 68.27, 71.36, 112.92, 121.74, 122.74, 132.32, 132.46, 156.93, 165.43; MS: [M + H]⁺ calc. 387.2642, obs. 387.2457 | 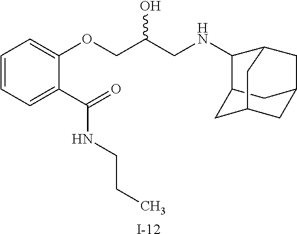 |
| 2-[3-(Adamantan-2-yl-methyl-amino)-2-hydroxy- propoxy]-N-benzyl-benzamide (I-13) ¹³C NMR: δ = 27.26, 27.43, 29.70, 30.05, 31.46, 31.66, 37.32, 37.60, 37.67, 38.79, 44.12, 56.19, 66.04, 67.99, 71.80, 113.09, 121.80, 122.37, 127.21, 128.08, 128.57, 132.47, 132.68, 139.05, 157.07, 165.31; MS: [M + H]⁺ calc. 449.2799, obs. 449.275 | 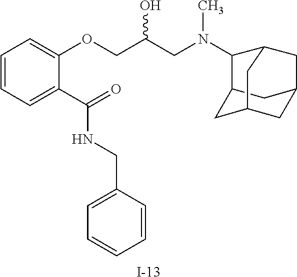 |
| 2-[3-(Adamantan-2-yl-methyl-amino)-2-hydroxy- propoxy]-benzoic acid methyl ester (INT-14) ¹³C NMR: δ = 27.40, 27.58, 29.81, 30.05, 31.57, 31.71, 37.50, 37.59, 37.84, 39.46, 52.12, 56.88, 67.06, 68.17, 72.32, 114.30, 120.47, 120.81, 131.87, 133.79, 158.93, 166.83; MS: [M + H]⁺: calc. 374.23315, obs. 374.2301 | 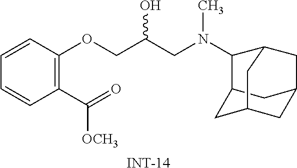 |
| 2-[3-(Adamantan-2-yl-methyl-amino)-2-hydroxy- propoxy]-benzoic acid (INT-15) ¹³C NMR (DMSO-d₆): δ = 26.60, 26.81, 28.99 (2x), 30.82, 30.85, 36.74, 36.82, 37.20, 39.78, 56.89, 66.85, 67.42, 71.81, 114.02, 120.29, 122.11, 130.75, 132.85, 157.62, 167.40; MS: [M + H]⁺: calc. 360.2169, obs. 360.2102 | 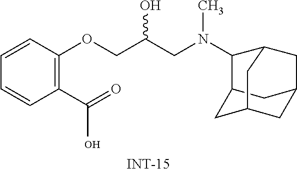 |
| 2-[3-(Adamantan-2-yl-methyl-amino)-2-hydroxy- propoxy]-N-(4-trifluoromethyl-benzyl)-benzamide (I-16) ¹³C NMR: δ = 27.24, 27.39, 29.73, 30.06, 31.43, 31.70, 37.30, 37.60, 37.63, 38.71, 43.53, 56.03, 65.95, 68.08, 71.89, 113.38, 122.01, 122.18, 124.37, 125.48, 129.40, 128.08, 132.54, 132.92, 143.36, 157.15, 165.57; MS: [M + H]⁺ calc. 517.2673, obs. 517.2543 | 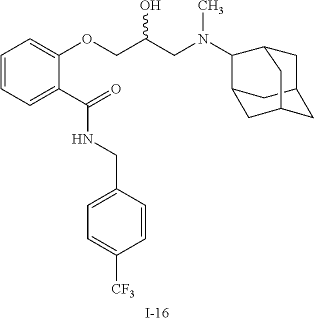 |
| 2-[3-(Adamantan-2-yl-propyl-amino)-2-hydroxy- propoxy]-N-benzyl-benzamide (I-17) ¹³C NMR: δ = 11.89, 16.81, 27.31, 27.44, 29.81, 30.35, 31.38, 31.71, 37.49, 37.60, 37.68, 44.12, 52.68, 52.85, 64.97, 66.80, 71.89, 113.15, 121.80, 122.40, 127.20, 128.07, 128.56, 132.49, 132.67, 139.06, 157.13, 165.33; MS: [M + H]⁺ calc. 477.3112, obs. 477.3120; | 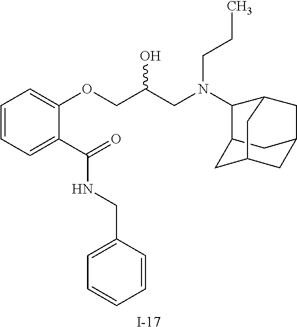 |
| 2-[3-(Adamantan-2-yl-propyl-amino)-2-hydroxy- propoxy]-N-(2-methoxy-benzyl)-benzamide (I-18) ¹³C NMR: δ = 11.88, 16.65, 27.33, 27.47, 29.85, 30.31, 31.41, 31.73, 37.50, 37.60, 37.70, 39.20, 52.56, 53.06, 55.62, 64.94, 66.97, 71.91, 110.45, 113.02, 120.72, 121.70, 122.67, 127.13, 128.46, 129.36, 132.48, 132.54, 157.09, 157.59, 165.38; MS: [M + H]⁺ calc. 507.3218, obs. 507.3194 | 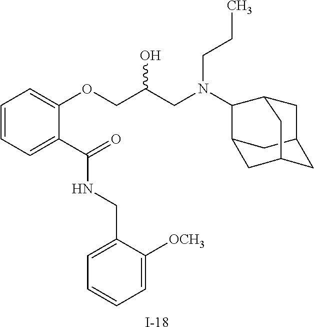 |
| 2-[3-(Adamantan-2-yl-pentyl-amino)-2-hydroxy- propoxy]-N-benzyl-benzamide (I-19) ¹³C NMR: δ = 14.27, 22.80, 23.10, 27.32, 27.45, 29.81, 29.93, 30.35, 31.41, 31.74, 37.50, 37.61, 37.69, 44.13, 50.50, 52.71, 64.79, 66.77, 71.91, 113.16, 121.83, 122.43, 127.20, 128.09, 128.58, 132.52, 132.68, 139.07, 157.14, 165.33; MS: [M + H]⁺ calc. 505.3425; obs. 505.3331 | 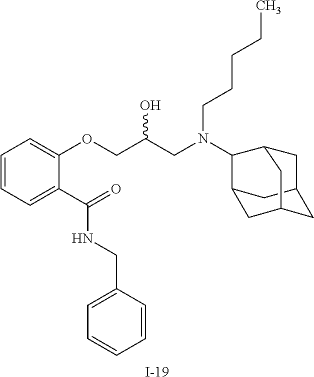 |
| 2-[3-(Adamantan-2-yl-cyclopropyl-amino)-2-hydroxy- propoxy]-N-benzyl-benzamide (I-20) ¹³C NMR: δ = 6.04, 8.17, 27.43, 27.54, 30.26, 30.98, 31.86, 32.03, 37.73, 37.82, 37.95, 38.95, 44.15, 57.58, 66.85, 71.32, 72.09, 113.13, 121.78, 122.37, 127.20, 128.13, 128.56, 132.45, 132.67, 139.00, 157.09, 165.33; MS: [M + H]⁺ calc. 475.2955, obs. 475.2864 | 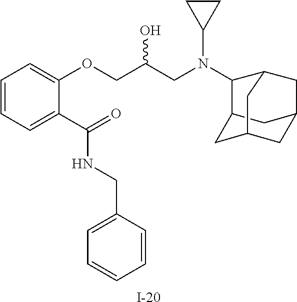 |
| 2-{3-[Adamantan-2-yl-(2-methoxy-benzyl)-amino]-2- hydroxy-propoxy}-N-benzyl-benzamide (I-21) ¹³C NMR: δ = 27.38, 27.55, 30.37, 30.66, 31.52, 31.75, 37.70, 37.72 (2x), 43.94, 50.17, 53.65, 55.25, 66.87, 67.35, 72.11, 110.72, 113.14, 120.49, 121.67, 122.29, 127.11, 127.17, 127.94, 128.60, 128.73, 131.13, 132.41, 132.62, 139.15, 157.11, 158.11, 165.38; MS: [M + H]⁺ calc. 555.3218, obs. 555.3172 | 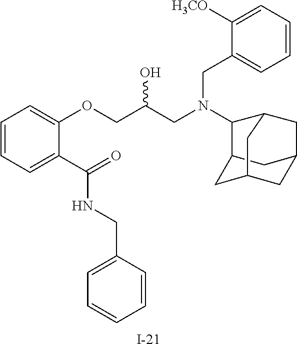 |
| 2-[3-(Adamantan-2-yl-phenethyl-amino)-2-hydroxy- propoxy]-N-benzyl-benzamide (I-22) ¹³C NMR: δ = 27.30, 27.43, 29.87, 29.95, 30.42, 31.38, 31.71, 37.49, 37.59, 37.65, 44.20, 52.48, 52.63, 64.57, 66.99, 71.73, 113.15, 121.89, 122.45, 126.30, 127.26, 128.13, 128.59, 128.69, 128.72, 132.54, 132.70, 138.99, 140.20, 157.09, 165.32; MS: [M + H]⁺ calc. 539.3268, obs. 539.3090 | 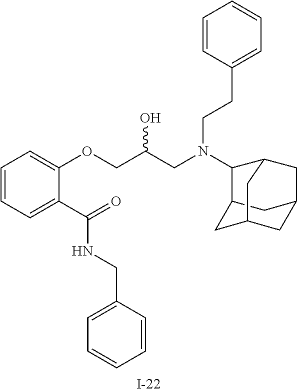 |
| 2-{3-[Adamantan-2-yl-(3-phenyl-propyl)-amino]-2- hydroxy-propoxy}-N-benzyl-benzamide (I-23) ¹³C NMR: δ = 25.24, 27.26, 27.40, 29.77, 30.29, 31.37, 31.68, 33.86, 37.43, 37.55, 37.62, 44.10, 49.83, 52.65, 64.74, 66.75, 71.78, 113.18, 121.80, 122.41, 126.09, 127.18, 128.06, 128.37, 128.52, 128.55, 132.47, 132.65, 139.04, 141.85, 157.10, 165.30; MS: [M + H]⁺ calc. 553.3425, obs. 553.3382 | 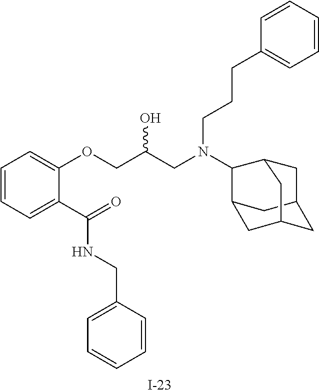 |
| 2-{3-[(Adamantan-1-ylmethyl)-amino]-2-hydroxy- propoxy}-N-(3-methyl-butyl)-benzamide (I-24) ¹³C NMR: δ = 22.71, 22.72, 26.11, 28.53, 33.66, 37.31, 38.36, 38.58, 40.95, 52.00, 62.41, 67.25, 71.24, 112.89, 121.74, 122.73, 132.33, 132.45, 156.94, 165.35; MS: [M + H]⁺ calc. 429.3112 obs. 429.3097 | 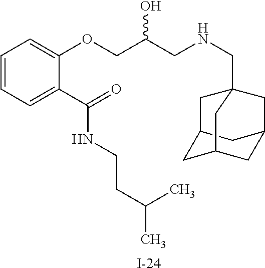 |
| 2-[3-(1-Adamantan-1-yl-ethylamino)-2-hydroxy- propoxy]-N-(3-methyl-butyl)-benzamide (I-25) ¹³C NMR: (2 diastereoisomers) δ = 13.67, 14.23, 22.63, 22.64, 26.05, 26.09, 28.61, 36.20, 36.51, 37.37, 38.33, 38.51, 38.53, 38.89, 49.33, 51.08, 61.61, 63.87, 67.25, 68.97, 71.18, 71.24, 112.84, 112.89, 121.62, 122.57, 122.62, 132.24, 132.41, 156.93, 156.97, 165.30, 165.33; [M + H]⁺ calc. 443.3268, obs. 443.3232 | 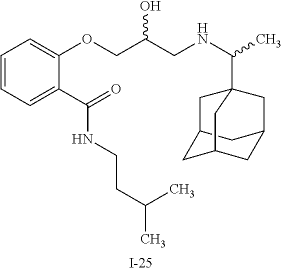 |
| 2-{3-[(Adamantan-1-ylmethyl)-amino]-2-hydroxy- propoxy}-N-(3-methyl-butyl)-5-prop-2-ynyloxy- benzamide (I-26) ¹³C NMR: δ = 22.66, 22.67, 26.06, 28.49, 33.61, 37.27, 38.39, 38.49, 40.90, 51.97, 56.63, 62.39, 67.29, 72.08, 75.65, 78.62, 114.76, 117.16, 120.11, 123.41, 151.81, 152.18, 164.79; MS: [M + H]⁺ calc. 483.3218, obs. 483.3223 | 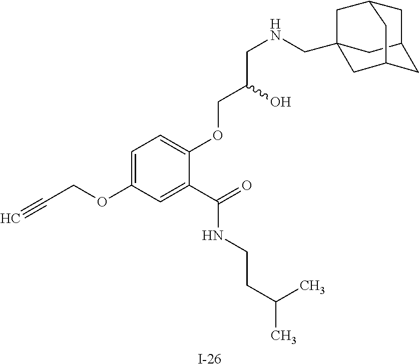 |
| 2-{3-[(Adamantan-1-ylmethyl)-amino]-2-hydroxy- propoxy}-N-benzyl-benzamide (I-27) ¹³C NMR: δ = 28.51, 33.53, 37.30, 40.86, 44.11, 51.94, 62.15, 67.03, 71.00, 112.93, 121.74, 122.31, 127.35, 128.22, 128.65, 132.39, 132.71, 139.05, 157.05, 165.35; MS: [M + H]⁺ calc. 449.2799, obs. 449.2597 | 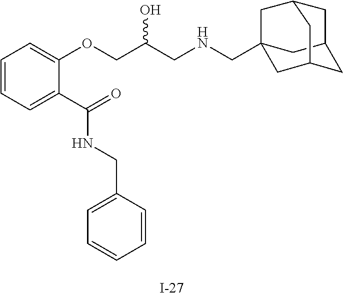 |
| 2-[3-(1-Adamantan-1-yl-ethylamino)-2-hydroxy- propoxy]-N-benzyl-benzamide (I-28) ¹³C NMR: (2 diastereoisomers) δ = 13.61, 14.28, 28.65, 36.12, 36.51, 37.43, 38.87, 44.10, 44.14, 49.12, 51.04, 61.50, 63.73, 67.07, 68.92, 70.81, 71.20, 112.91, 113.03, 121.77, 121.82, 122.39, 127.32, 127.38, 128.05, 128.29, 128.66, 128.68, 132.46, 132.49, 132.73, 139.06, 139.13, 157.08, 157.11, 165.33, 165.38; MS: [M + H]⁺ calc. 463.2955, obs. 463.2801 | 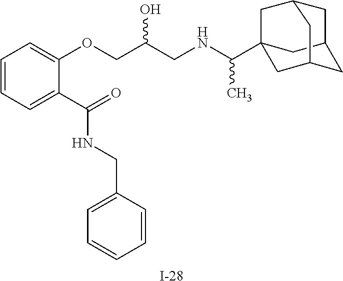 |
| 2-{3-[(Adamantan-1-ylmethyl)-amino]-2-hydroxy- propoxy}-N-(2-methoxy-benzyl)-benzamide (I-29) ¹³C NMR: δ = 28.40, 33.39, 37.12, 39.30, 40.64, 52.45, 55.69, 62.05, 66.90, 71.32, 110.60, 113.09, 120.77, 121.78, 122.85, 126.99, 128.70, 129.54, 132.16, 132.58, 156.88, 157.67, 165.52; MS: [M + H]⁺ calc. 479.2905, obs. 479.2721 | 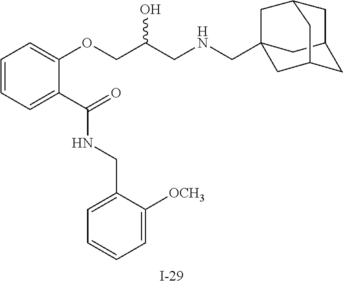 |
| 2-{3-[(Adamantan-1-ylmethyl)-amino]-2-hydroxy- propoxy}-N-(4-trifluoromethyl-benzyl)-benzamide (I-30) ¹H NMR: δ = 1.43-1.48 (6H), 1.59-1.65 (3H), 1.68-1.77 (3H), 1.96 (3H), 2.13-2.24 (2H), 2.58 (1H), 2.70 (1H), 3.88-3.95 (1H), 3.99 (1H), 4.18 (1H), 4.64-4.74 (2H), 6.94 (1H), 7.09 (1H), 7.42 (1H), 7.48-7.50 (2H), 7.56- 7.58 (2H), 8.18 (1H), 8.80 (1H); ¹³C NMR: δ = 28.47, 33.53, 37.23, 40.82, 43.42, 51.87, 62.31, 67.09, 71.34, 113.29, 121.96, 122.23, 124.33, 125.51, 128.06, 129.45, 132.43, 132.91, 143.43, 157.11, 165.64 | 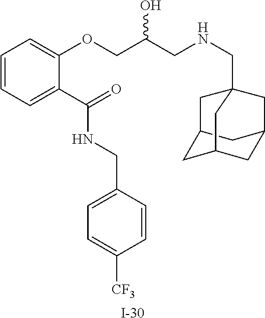 |
| 2-{3-[(Adamantan-1-ylmethyl)-amino]-2-hydroxy- propoxy}-N-phenethyl-benzamide (I-31) ¹³C NMR: δ = 28.54, 33.66, 35.87, 37.32, 40.96, 41.32, 52.06, 62.52, 67.35, 71.38, 113.17, 121.79, 122.57, 126.35, 128.59, 129.03, 132.29, 132.59, 139.85, 157.04, 165.47; MS: [M + H]⁺ calc. 463.2955, obs. 463.2877 | 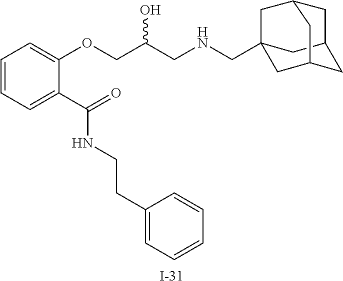 |
| 2-{3-[(Adamantan-1-ylmethyl)-amino]-2-hydroxy- propoxy}-N-(3-phenyl-propyl)-benzamide (I-32) ¹³C NMR: δ = 28.52, 31.34, 33.59, 33.61, 37.29, 39.73, 40.88, 52.04, 62.45, 67.31, 71.32, 112.98, 121.78, 122.70, 125.97, 128.51, 132.34, 132.52, 141.99, 156.96, 165.50; MS: [M + H]⁺ calc. 477.3112, obs. 477.2980 | 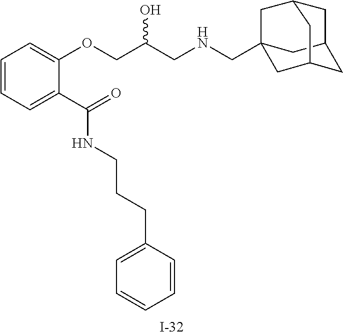 |
| 2-{3-[(Adamantan-1-ylmethyl)-amino]-2-hydroxy- propoxy}-5-chloro-N-phenethyl-benzamide (I-33) ¹³C NMR: δ = 28.33, 33.46, 35.55, 37.11, 40.75, 41.22, 51.80, 62.32, 67.01, 71.70, 114.59, 123.78, 126.21, 126.92, 128.41, 128.79, 131.74, 131.99, 139.44, 155.40, 163.97; MS: [M + H]⁺ calc. 497.2566, obs. 497.2529 | 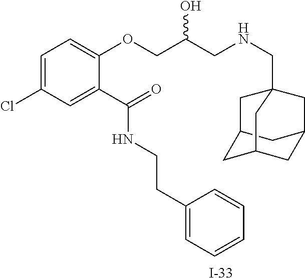 |
| 2-{3-[(Adamantan-1-ylmethyl)-amino]-2-hydroxy- propoxy}-5-chloro-N-naphthalen-1-ylmethyl- benzamide (I-34) ¹³C NMR: δ = 28.53, 33.48, 37.34, 40.82, 42.52, 51.64, 62.02, 66.73, 71.15, 114.34, 123.61, 124.13, 125.63, 126.13, 126.73, 127.17, 128.59, 128.78, 131.86, 132.19, 132.36, 133.91, 134.11, 155.60, 163.83 (C_{q}-5-Cl not recorded); MS: [M + H]⁺ calc. 533.2566, obs. 533.2559 | 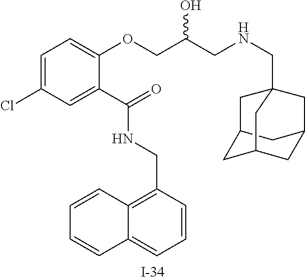 |
| 2-[3-(1-Adamantan-1-yl-ethylamino)-2-hydroxy- propoxy]-5-chloro-N-naphthalen-1-ylmethyl-benzamide (I-35) ¹³C NMR: (2 diastereoisomers) δ = 13.50, 14.20, 28.65, 28.62, 36.43, 36.01, 37.43, 37.40, 38.82, 38.79, 42.53, 42.38, 50.61, 48.92, 63.56, 61.48, 68.58, 66.85, 71.53, 70.83, 114.30, 114.43, 123.57, 123.72, 123.97, 124.18, 125.59, 125.65, 126.07, 126.13, 126.66, 126.70, 126.73, 127.25, 128.45, 128.62, 128.77, 128.82, 131.78, 131.89, 132.14, 132.22, 132.35 (2x), 133.95, 134.07, 134.15, 133.90, 155.61, 155.65, 163.82, 163.92 (C_{q}-5-Cl not recorded); MS: [M + H]⁺ calc. 547.2723, obs. 547.2747 | 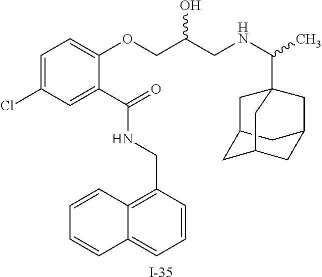 |
| 2-[3-(Adamantan-1-ylmethyl-methyl-amino)-2- hydroxy-propoxy]-N-benzyl-benzamide (I-36) ¹H NMR: δ = 1.45 (6H), 1.56-1.63 (3H), 1.66-1.73 (3H), 1.91-1.97 (3H), 1.99-2.07 (2H), 2.21 (3H), 2.33- 2.42 (2H), 3.83-3.89 (1H), 3.98 (1H), 4.09 (1H), 4.58 (1H), 4.72 (1H), 6.92 (1H), 7.08 (1H), 7.22-7.27 (1H), 7.29-7.35 (2H), 7.36-7.43 (3H), 8.22 (1H), 8.61 (1H). ¹³C NMR: δ = 28.53, 34.92, 37.23, 41.40, 44.16, 45.84, 63.36, 66.40, 71.42, 72.16, 113.04, 121.85, 122.39, 127.30, 128.11, 128.64, 132.53, 132.71, 139.06, 157.07, 165.33 | 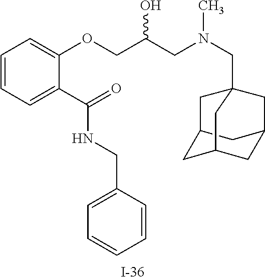 |
| 2-[3-(Adamantan-1-ylmethyl-methyl-amino)-2- hydroxy-propoxy]-N-(4-fluoro-phenyl)-benzamide (I-37) ¹HNMR: δ = 1.46-1.50 (6H), 1.54-1.62 (3H), 1.66-1.73 (3H), 1.89-1.95 (3H), 2.09-2.17 (2H), 2.21 (3H), 2.49- 2.58.58 (2H), 4.05 (1H), 4.10 (1H), 4.26 (1H), 6.97 (1H), 6.99-7.05 (2H), 7.13 (1H), 7.46 (1H), 7.76-7.82 (2H), 8.26 (1H), 10.19 (1H); ¹³C NMR: δ = 28.52, 34.91, 37.18, 41.49, 46.03, 63.09, 66.49, 71.24, 72.22, 112.71, 115.47, 122.05, 122.23, 122.47, 132.72, 133.18, 135.41, 156.69, 159.31, 163.44 | 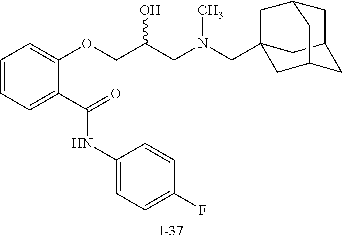 |
| 2-{3-[(Adamantan-1-ylmethyl)-amino]-2-hydroxy- propoxy}-N-(2-fluoro-phenyl)-benzamide (I-38) ¹³C NMR: δ = 28.56, 33.68, 37.34, 40.91, 52.51, 62.63, 67.50, 72.08, 112.98, 114.86, 121.95, 121.97, 122.78, 124.25, 124.78, 127.17, 132.75, 133.59, 153.08, 156.94, 163.55; MS: [M + H]⁺ calc. 453.2548, obs. 453.251 | 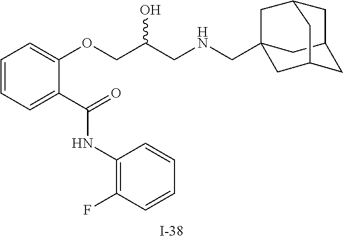 |
| 2-{3-[(Adamantan-1-ylmethyl)-amino]-2-hydroxy- propoxy}-N-(2,4-difluoro-phenyl)-benzamide (I-39) ¹H NMR: δ = 1.50-1.51 (6H), 1.60-1.67 (3H), 1.69-1.77 (3H), 1.98 (3H), 2.22-2.32 (2H), 2.72 (1H), 2.87 (1H), 4.09-4.16 (1H), 4.17-4.24 (2H), 6.84-6.93 (2H), 7.05 (1H), 7.13 (1H), 7.48 (1H), 8.26 (1H), 8.40-8.48 (1H), 10.05 (1H); ¹³C NMR: δ = 28.54), 33.67, 37.31, 40.91, 52.36, 62.60, 67.43, 72.02, 103.59, 111.28, 113.02, 121.76, 122.00, 123.42, 123.91, 132.72, 133.65, 154.42, 156.96, 158.66, 163.57 | 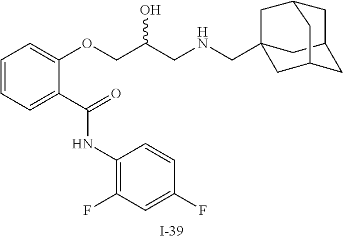 |
| 2-{3-[(Adamantan-1-ylmethyl)-amino]-2-hydroxy- propoxy}-N-p-tolyl-benzamide (I-40) ¹³C NMR: δ = 21.05, 28.54, 33.65, 37.31, 40.90, 51.86, 62.45, 67.37, 71.22, 112.69, 120.56, 121.92, 122.79, 129.50, 132.64, 132.99, 133.50, 136.50, 156.69, 163.37; MS: [M + H]⁺ calc. 449.2799, obs. 449.2647 | 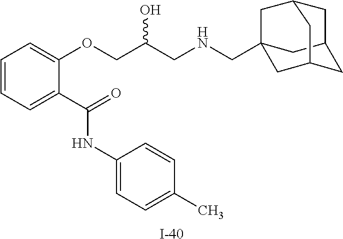 |
| 2-[3-(1-Adamantan-1-yl-ethylamino)-2-hydroxy- propoxy]-N-p-tolyl-benzamide (I-41) ¹³C NMR: (two diastereoisomers) δ = 13.73, 14.27, 21.05, 28.65, 28.68, 37.41, 36.25, 38.87, 38.93, 49.23, 50.88, 61.71, 63.88, 67.32, 68.98, 70.95, 71.37, 112.63, 112.68, 120.51, 120.70, 121.91, 122.82 (C_{q} HMBC), 129.47, 129.52, 132.65, 132.99, 133.70 (C_{q} HMBC), 136.43 (C_{q} HMBC), 156.84 (C_{q} HMBC), 163.21 (C_{q} HMBC); MS: [M + H]⁺ calc. 463.2955, obs. 463.2893 | 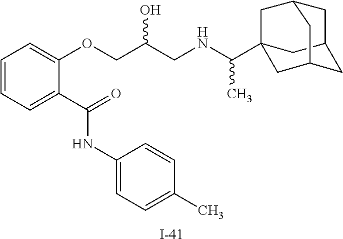 |
| 2-{3-[(Adamantan-1-ylmethyl)-amino]-2-hydroxy- propoxy}-N-(3-trifluoromethyl-phenyl)-benzamide (I-42) (I-42) ¹H NMR: δ = 1.48-1.53 (6H), 1.60-1.67 (3H), 1.68-1.77 (3H), 1.98 (3H), 2.23-2.33 (2H), 2.75 (1H), 2.84 (1H), 4.05 (1H), 4.09-4.16 (1H), 4.31 (1H), 6.99 (1H), 7.14 (1H), 7.34 (1H), 7.44 (1H), 7.48 (1H), 8.09 (1H), 8.14 (1H), 8.26 (1H), 10.39 (1H); ¹³C NMR: δ = 28.51, 33.60, 37.28, 40.83, 51.55, 62.23, 67.00, 71.34, 112.78, 117.35, 120.38, 122.10, 122.20, 123.56, 129.44, 131.29, 132.74, 133.48, 139.67, 156.77, 163.79, (C_{q} of CF₃ not recorded) | 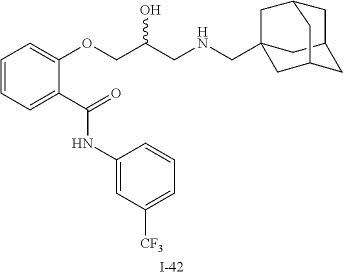 |
| 2-[3-(Adamantan-2-yl-methyl-amino)-2-hydroxy- propoxy]-N-[4-(3-trifluoromethyl-3H-diazirin-3-yl)- benzyl]-benzamide (I-43) ¹³C NMR: δ = 27.23, 27.39, 28.48, 29.72, 30.05, 31.44, 31.70, 37.29, 37.59, 37.63, 38.71, 43.40, 56.03, 65.95, 68.05, 71.83, 113.33, 121.96, 122.17, 122.28, 126.70, 127.84, 128.24, 132.50, 132.87, 141.15, 157.12, 165.5;. MS: [M + H]⁺ calc. 557.27396, obs. 557.2603 | 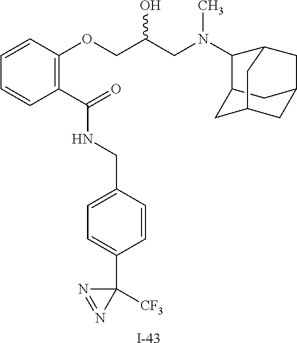 |
| 2-{2-Hydroxy-3-[4-(3-trifluoromethyl-3H-diazirin-3- yl)-benzylamino]-propoxy}-N-(3-methyl-butyl)-5-prop- 2-ynyloxy-benzamide (I-44) ¹³C NMR: δ = 22.63, 26.06, 28.37, 38.38, 38.53, 51.14, 53.23, 56.66, 68.18, 72.32, 75.74, 78.59, 115.00, 117.12, 120.04, 122.26, 123.73, 126.885, 128.36, 128.64, 141.36, 151.61, 152.31, 165.02; MS: [M + H]⁺ calc. 533.2370, obs. 533.2423 | 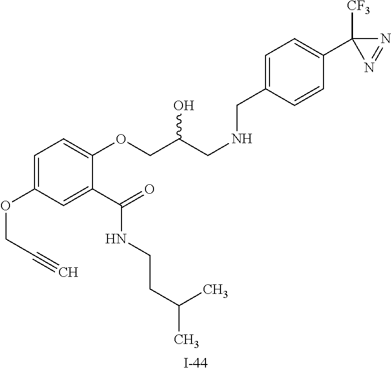 |
| N-(4-Fluoro-phenyl)-2-{2-hydroxy-3-[4-(3- trifluoromethyl-3H-diazirin-3-yl)-benzylamino]- propoxy}-5-prop-2-ynyloxy-benzamide (I-45) ¹H NMR: δ = 2.66 (1H), 2.75 (1H), 2.84 (1H), 3.81- 3.85 (2H), 3.97-4.10 (1H), 4.83 (2H), 6.98-7.07 (4H), 7.14-7.16 (2H), 7.35-7.37 (2H), 7.63-7.68 (2H), 7.80 (1H), 9.85 (1H); ¹³C NMR: δ = 28.31 (HMBC), 51.19, 53.24, 58.41, 68.53, 71.24, 77.21, 79.72, 115.34, 115.76, 116.93, 120.34, (C_{q} of CF₃ not recorded), 122.00, 123.47, 126.82, 128.16, 128.72, 134.54, 141.57, 149.93, 154.08, 159.50, 162.57 | 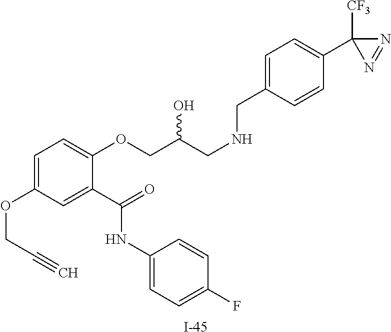 |
| N-(4-Fluoro-phenyl)-2-[2-hydroxy-3-(4-phenyl- piperidin-1-yl)-propoxy]-benzamide (I-46) ¹H NMR: δ = 1.77 (1H), 1.78 (1H), 1.82-1.83 (2H), 2.15 (1H), 2.45 (1H), 2.50 (1H), 2.54 (1H), 2.66 (1H), 2.79-2.86 (1H), 3.10-3.18 (1H), 4.05 (2H), 4.18-4.25 (1H), 4.34 (1H), 6.96 6.98 (1H), 7.00-7.06 (2H), 7.13 (1H), 7.20-7.26 (3H), 7.29-7-35 (2H), 7.46 (1H), 7.76- 7.82 (2H), 8.26 (1H), 10.15 (1H); ¹³C NMR: δ = 33.47, 33.82, 42.41, 52.89, 56.32, 60.08, 65.27, 71.05, 112.72, 115.49, 122.04, 122.13, 122.50, 126.48, 126.89, 128.65, 132.67, 133.21, 135.12, 145.89, 156.63, 159.28, 163.47 | 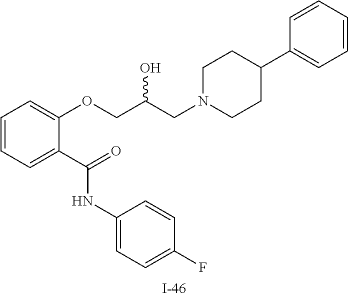 |
| 2-[3-(4-Benzyl-piperidin-1-yl)-2-hydroxy-propoxy]-N- (4-fluoro-phenyl)-benzamide (I-47) ¹H NMR: δ = 1.22-1.35 (2H), 1.49-1.60 (1H), 1.60-1.70 (2H), 1.94 (1H), 2.26 (1H), 2.41 (1H), 2.55 (2H), 2.59 (1H), 2.63-2.70 (1H), 2.96-3.02 (1H), 4.02 (1H), 4.13- 4.20 (1H), 4.30 (1H), 6.96 (1H), 6.99-7.07 (2H), 7.10- 7.16 (3H), 7.17-7.22 (1H), 7.24-7.32 (2H), 7.45 (1H), 7.73-7-80 (2H), 8.25 (1H), 10.12 (1H); ¹³C NMR: δ = 32.24, 32.59, 37.85, 43.20, 52.51, 55.82, 59.98, 65.16, 71.03, 112.70, 115.49, 122.04, 122.13, 122.51, 126.08, 128.39, 129.25, 132.68, 133.19, 135.12, 140.50, 156.64, 159.29, 163.46 | 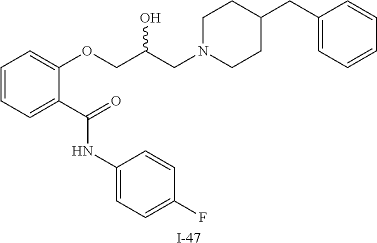 |
| 2-[3-(4-Benzhydryl-piperazin-1-yl)-2-hydroxy- propoxy]-N-(4-fluoro-phenyl)-benzamide (I-48) ¹H NMR: δ = 2.22-2.54 (dyn, 6H), 2.46 (1H), 2.63 (1H), 2.67-2.79 (dyn, 2H), 3.80-4.00 (br, 1H), 4.02 (1H), 4.13-4.20 (1H), 4.26 (1H), 4.30 (1H), 6.95 (1H), 6.97-7,03 (2H), 7.12 (1H), 7.16-7.22 (2H), 7.26-7.31 (4H), 7.38-7.42 (4H), 7.45 (1H), 7.71-7.77 (2H), 8.24 (1H), 10.11 (1H); ¹³C NMR: δ = 51.98, 53.57 (br), 59.73, 65.09, 70.97, 76.20, 112.70, 115.50, 122.04, 122.08, 122.48, 127.19, 128.05, 128.67, 132.67, 133.20, 135.08, 142.52, 142.59, 156.58, 159.24, 163.42 | 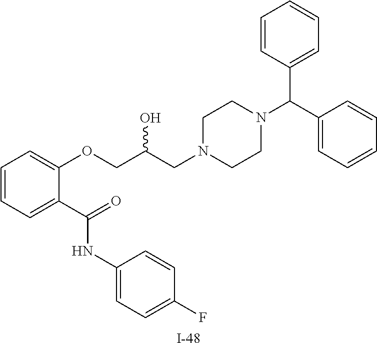 |
| 2-[3-(4-Benzoyl-benzylamino)-2-hydroxy-propoxy]-N- benzyl-benzamide (I-49) ¹³C NMR: δ = 44.20, 51.17, 53.33, 68.03, 71.13, 113.01, 121.92, 122.51, 127.49, 127.90, 128.30, 128.46, 128.76, 130.13, 130.57, 132.32, 132.58, 132.77, 136.78, 137.79, 139.03, 144.64, 156.91, 165.41, 196.45; MS: [M + H]⁺ calc. 495.2279, obs. 495.2353 | 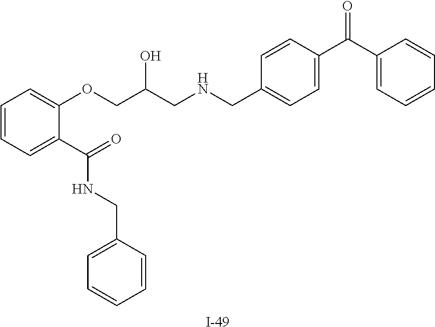 |
| N-Benzyl-2-[2-hydroxy-3-(3-phenyl-propylamino)- propoxy]-benzamide (I-50) ¹H NMR: δ = 1.50-1.93 (2H), 2.41-2.92 (6H), 3.78-3.55 (1H), 3.85-3.92 (1H), 3.97-4.03 (1H), 4.51-4.64 (2H), 6.87 (1H), 7.02-7.43 (11H), 7.39 (1H), 8.08-8.15 (1H), 8.25-8.36 (1H); ¹³C NMR: δ = 28.61, 33.49, 43.94, 55.15, 57.35, 67.27, 71.37, 113.26, 121.96, 122.62, 127.31, 127.33, 127.88, 128.35, 128.62, 128.68, 132.10, 132.77, 138.94, 141.55, 156.90, 165.60 | 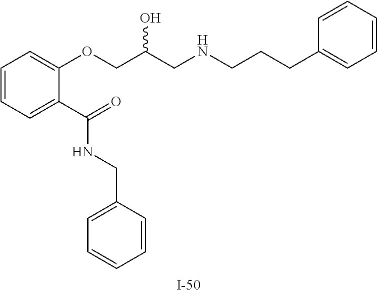 |
| 2-{3-[4-(2,3-Dimethyl-phenyl)-piperazin-1-yl]-2- hydroxy-propoxy}-N-(4-methoxy-benzyl)-benzamide (I-51) ¹³C NMR: δ = 14.02, 20.73, 43.60, 52.27, 53.69, 55.33, 60.21, 65.11, 71.04, 112.93, 113.99, 116.68, 121.79, 122.34, 125.25, 125.98, 129.41, 131.05, 131.33, 132.42, 132.68, 138.14, 151.40, 156.97, 158.95, 165.21; MS: [M + H]⁺ calc. 504.2784, obs. 504.2716 | 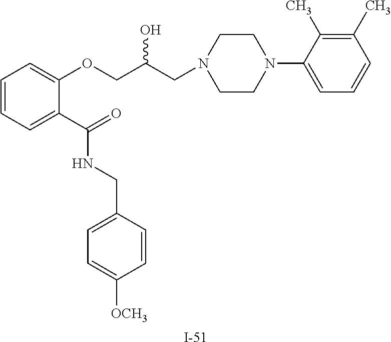 |
| N-Benzyl-2-(3-cyclododecylamino-2-hydroxy- propoxy)-benzamide (I-52) ¹³C NMR: δ = 21.02, 21.17, 23.14, 23.23, 23.32, 23.41, 24.40, 24.92, 29.67, 30.04, 44.13, 48.69, 54.74, 67.93, 71.16, 113.01, 121.80, 122.38, 127.36, 128.17, 128.67, 132.41, 132.72, 139.06, 157.05, 165.35; MS: [M + H]⁺ calc. 467.3268, obs. 467.3166 | 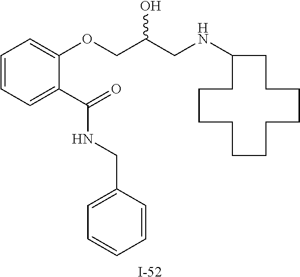 |
| N-Benzyl-2-(3-cyclooctylamino-2-hydroxy-propoxy)- benzamide (I-53) ¹³C NMR: δ = 23.83, 23.98, 25.71, 27.34, 27.41, 32.14, 32.69, 44.03, 48.97, 57.80, 67.85, 71.25, 113.00, 121.71, 122.32, 127.29, 128.06, 128.62, 132.23, 132.70, 138.98, 156.99, 165.42; MS: [M + H]⁺ calc. 411.26420, obs. 411.2567 | 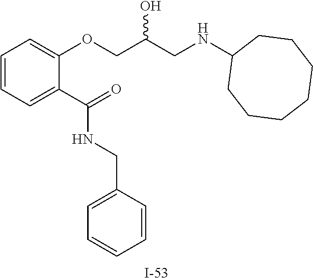 |
| 2-(2-Hydroxy-3-propylamino-propoxy)-N-p-tolyl- benzamide (I-54) ¹³C NMR: δ = 11.74, 21.03, 23.39, 51.31, 51.67, 67.75, 71.35, 112.70, 120.53, 121.93, 122.79, 129.48, 132.59, 133.00, 133.54, 136.45, 156.64, 163.39; MS: [M + H]⁺ calc. 343.2016, obs. 343.185 | 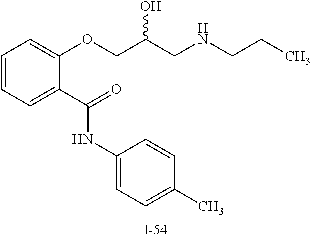 |
| 2-{3-[(Adamantan-1-ylmethyl)-amino]-2-hydroxy- propoxy}-N-(4-fluoro-phenyl)-benzamide (I-55) ¹³C NMR: δ = 28.52, 33.66, 37.29, 40.92, 51.76, 62.48, 67.31, 71.30, 112.76, 115.51, 122.03, 122.16, 122.51, 132.68, 133.19, 135.16, 156.71, 160.49, 163.46; MS: [M + H]⁺ calc. 453.25480, obs. 453.2427 | 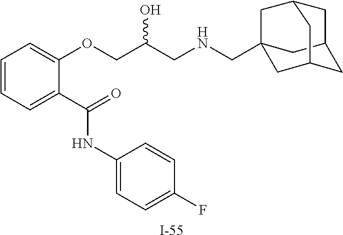 |
| 2-(3-Butylamino-2-hydroxy-propoxy)-N-(3-methyl- butyl)-benzamide (I-56) ¹³C NMR: δ = 14.03, 20.42, 22.62, 26.06, 32.09 38.33, 38.51, 49.50 51.65, 67.55, 71.44, 112.93, 121.68, 122.75, 132.05, 132.45, 156.85, 165.51; MS: [M + H]⁺ calc. 337.24860, obs. 337.2349 | 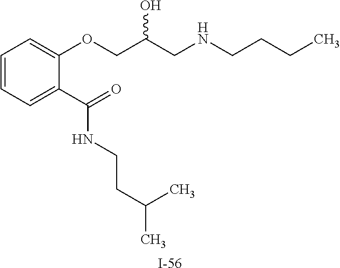 |
| 2-(3-Dibutylamino-2-hydroxy-propoxy)-N-(3-methyl- butyl)-benzamide (I-57) ¹³C NMR: δ = 14.16, 20.69, 22.66, 26.09, 29.47 38.37, 38.54, 54.09, 57.00, 65.78, 71.40, 112.84, 121.75, 122.66, 132.42, 132.43, 156.96, 165.28; MS: [M + H]⁺ calc. 393.31120, obs. 393.3067 | 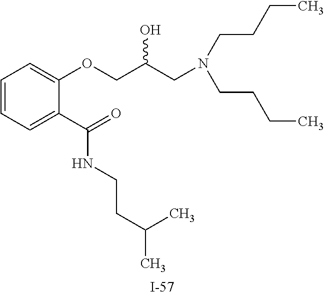 |
| 2-[2-Hydroxy-3-(3-methyl-butylamino)-propoxy]-N-(3- methyl-butyl)-benzamide (I-58) ¹³C NMR: δ = 22.63, 22.73, 22.77, 26.06, 26.16, 38.32, 38.53, 39.32, 48.04, 51.73, 67.70, 71.45, 112.93, 121.71, 122.74, 132.15, 132.45, 156.88, 165.45; MS: [M + H]⁺ calc. 351.26420, obs. 351.252 | 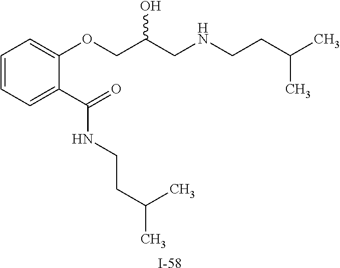 |
| 2-(3-Benzylamino-2-hydroxy-propoxy)-N-(3-methyl- butyl)-benzamide (I-59) ¹³C NMR: δ = 22.68, 26.09, 38.32, 38.58, 51.08, 53.90, 68.04, 71.41, 112.98, 121.80, 122.85, 127.50, 128.20, 128.75, 132.21, 132.45, 139.76, 156.84, 165.43; MS: [M + H]⁺ calc. 371.2329, obs. 371.2334 | 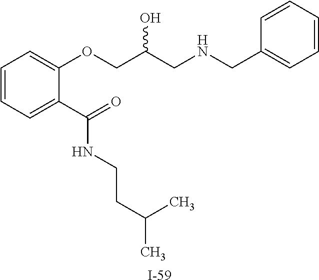 |
| 2-[2-Hydroxy-3-(4-trifluoromethyl-benzylamino)- propoxy]-N-(3-methyl-butyl)-benzamide (I-60) ¹³C NMR: δ = 22.62, 26.04, 38.29, 38.52, 51.33, 53.40, 68.29, 71.55, 113.09, 121.79, 122.89, 125.60, 128.37, 129.69, 131.97, 132.49, 143.92, 156.80, 165.59; MS: [M + H]⁺ calc. 439.2203, obs. 439.2243 | 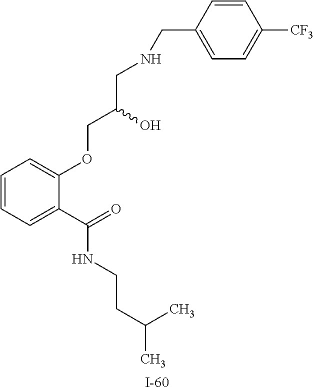 |
| 2-(3-Dibutylamino-2-hydroxy-propoxy)-N-(4-fluoro- phenyl)-benzamide (I-61) ¹H NMR: δ = 0.88 (6H), 1.18-1.33 (4H), 1.33-1.45 (4H), 2.38-2.46 (2H), 2.49-2.64 (4H), 4.03 (1H), 4.07- 4.14 (1H), 4.28 (1H), 6.96 (1H), 6.98-7.04 (2H), 7.12 (1H), 7.45 (1H), 7.75-7.81 (2H), 8.25 (1H), 10.18 (1H); ¹³C NMR: δ = 14.09, 20.63, 29.41, 54.09, 56.60, 65.80, 71.23, 112.67, 115.42, 121.98, 122.21, 122.46, 132.65, 133.17, 135.16, 156.71, 159.29, 163.47 | 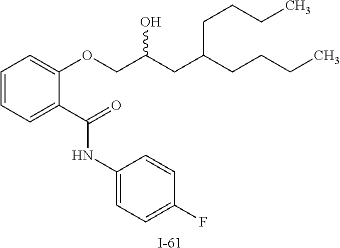 |
| 2-[3-(2-Adamantan-1-yl-ethylamino)-2-hydroxy- propoxy]-N-benzyl-benzamide (I-62) ¹³C NMR: δ = 17.13, 29.66, 36.71, 40.20, 42.88, 44.05, 50.00, 55.48, 66.15, 72.00, 113.21, 121.81, 122.41, 127.16, 127.96, 128.57, 132.53, 132.68, 139.11, 157.19, 165.36; MS: [M + H]⁺ calc. 463.2955, obs. 463.2854 | 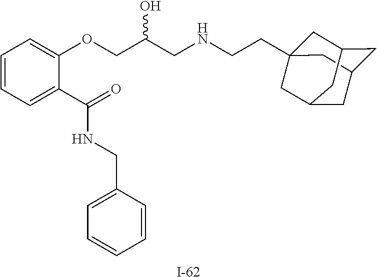 |
| N-Benzyl-2-{2-hydroxy-3-[(4-methoxy-3-prop-2- ynyloxy-benzyl)-propyl-amino]-propoxy}-benzamide (I-63) ¹³C NMR: δ = 11.85, 20.24, 29.84, 44.01, 56.02, 56.07, 56.42, 56.91, 58.32, 65.89, 71.38, 75.97, 78.76, 111.55, 112.93, 115.36, 121.83, 122.27, 122.64, 127.30, 127.96, 128.65, 130.93, 132.48, 132.73, 139.04, 146.76, 149.11, 156.96, 165.34; MS: [M + H]⁺ calc. 517.2697, obs. 517.2691 | 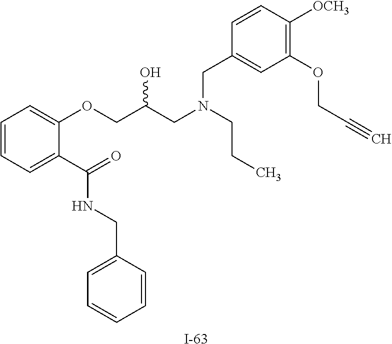 |
| 2-[3-(Adamantan-2-yl-methyl-amino)-2-hydroxy- propoxy]-N-(4-tert-butyl-benzyl)-benzamide (I-64) ¹H NMR: δ = 1.30 (9H), 1.39-2.35 (19H), 2.55-2.78 (1H), 3.95-4.15 (3H), 4.48-4.58 (1H), 4.64-4.74 (1H), 6.96 (1H), 7.07 (1H), 7.30-7.35 (4H), 7.40 (1H), 8.18 (1H), 8.56 (1H); MS: [M + H]⁺ calc. 505.3425, obs. 505.3249 | 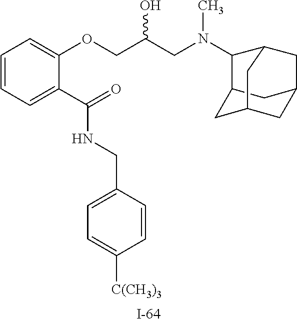 |
| 2-(3-Allylamino-2-hydroxy-propoxy)-N-(2-allyl- phenyl)-benzamide (I-65) ¹H NMR: δ = 2.39-2.64 (2H, br), 2.69 (1H), 2.85 (1H), 3.16-3.29 (2H), 3.47 (2H), 4.04-4.12 (1H), 4.14 (1H), 4.26 (1H), 4.98-5.18 (4H), 5.74-5.87 (1H), 5.93-6.05 (1H), 7.04 (1H), 7.12 (1H), 7.17 (1H), 7.21-7.30 (2H), 7.46 (1H), 7.80 (1H), 8.18 (1H), 9.42 (1H); ¹³C NMR: δ = 36.06, 50.84, 52.11, 67.77, 72.10, 113.38, 116.54, 116.81, 122.01, 123.05, 125.56, 125.89, 127.12, 129.84, 132.44, 132.93, 133.07, 136.05, 136.09, 136.39, 156.87, 164.25 | 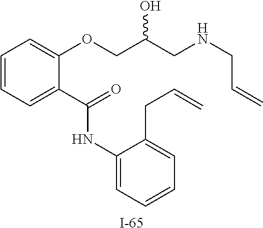 |
| 2-{3-[(Adamantan-1-ylmethyl)-amino]-2-hydroxy-pro- poxyl-5-chloro-N-(4-fluoro-phenyl)-benzamide (I-66) ¹H NMR: δ = 1.45-1.55 (6H), 1.60-1.67 (3H), 1.70-1.79 (3H), 1.99 (3H), 2.22-2.33 (2H), 2.73 (1H), 2.85 (1H), 4.03 (1H), 4.07-4.13 (1H), 4.27 (1H), 6.92 (1H), 6.99- 7.06 (2H), 7.40 (1H), 7.74-7.79 (2H), 8.22 (1H), 10.11 (1H); ¹³C NMR: δ = 28.49, 33.64, 37.26, 40.89, 51.63, 62.41, 67.13, 71.78, 114.32, 115.58, 122.24, 123.99, 127.47, 132.34, 132.76, 134.83, 155.20, 159.43, 162.11 | 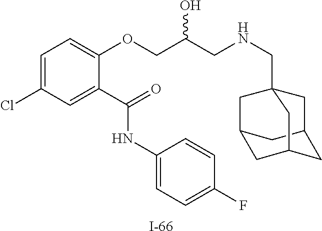 |
| 2-[2-Hydroxy-3-(4-phenyl-piperidin-1-yl)-propoxy]-N- (3-trifluoromethyl-phenyl)-benzamide (I-67) ¹H NMR: δ = 1.74-1.94 (4H), 2.18 (1H), 2.42-2.59 (3H), 2.69 (1H), 2.86-2.92 (1H), 3.15-3.22 (1H), 4.06 (1H), 4.24-4.35 (2H), 4.35-4.55 (1H), 6.97 (1H), 7.14 (1H), 7.20-7.25 (3H), 7.30-7.37 (3H), 7.42-7.50 (2H), 8.10 (1H), 8.16 (1H), 8.25 (1H), 10.37 (1H); ¹³C NMR: δ = 33.15, 33.43, 42.27, 52.88, 56.25, 60.07, 65.20, 71.27, 112.73, 117.25, 120.38, 122.08, 122.17, 123.47, 124.08, 126.48, 126.88, 128.63, 129.46, 131,23, 132.63, 133.50, 139.60, 145.74, 156.65, 163.82 | 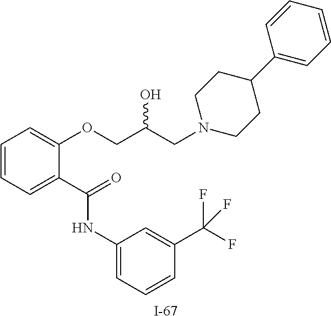 |
| 2-[3-(4-Benzyl-piperidin-1-yl)-2-hydroxy-propoxy]-N- (3-trifluoromethyl-phenyl)-benzamide (I-68) ¹H NMR: δ = 1.24-1.36 (2H), 1.50-1.59 (1H), 1.59-1.71 (2H), 1.97 (1H), 2.27 (1H), 2.43 (1H), 2.52 2.60 (3H), 2.67-2.74 (1H), 2.97-3.04 (1H), 3.99 (1H), 4.15-4.23 (1H), 4.27 (1H), 6.93 (1H), 7.11 (1H), 7.13-7.17 (2H), 7.18-7.24 (1H), 7.27-7.32 (2H), 7.33-7.37 (1H), 7.40- 7.47 (2H), 8.07 (br, 1H), 8.18 (br, 1H), 8.24 (1H), 10.39 (br, 1H); ¹³C NMR: δ = 32.04, 32.29, 37.69, 43.08, 52.47, 55.65, 59.85, 65.10, 71.26, 112.70, 117.20, 120.28, 121.94, 122.01, 123.40, 124.22, 125.98, 128.30, 129.16, 129.39, 131.15, 132.51, 133.44, 139.58, 140.46, 156.64, 163.76 | 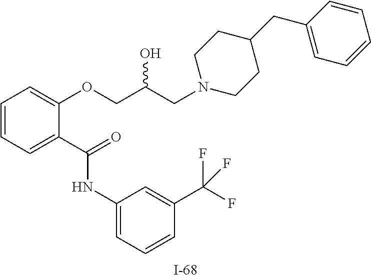 |
| 2-[3-(4-Benzhydryl-piperazin-1-yl)-2-hydroxy-prop- oxyl-N-(3-trifluoromethyl-phenyl)-benzamide (I-69) ¹H NMR: δ = 2.34-2.48 (6H), 2.49 (1H), 2.63 (1H), 2.69-2.79 (2H), 4.04 (1H), 4.18-4.25 (1H), 4.26 (1H), 4.33 (1H), 6.97 (1H), 7.14 (1H), 7.16-7.22 (2H), 7.24- 7.31 (4H), 7.33 (1H), 7.39-7.44 (5H), 7.48 (1H), 8.06 (1H), 8.09 (1H), 8.26 (1H), 10.32 (1H); ¹³C NMR: δ = 51.92, 53.58, 59.72, 65.05, 71.15, 76.19, 112.72, 117.25, 120.40, 122.16, 122.19, 123.43, CF₃ not recorded, 127.17, 128.05, 128.67, 129.47, C_{q}CF₃ not recorded, 132.74, 133.50, 139.57, 142.62, 142.66, 156.66, 163.76 | 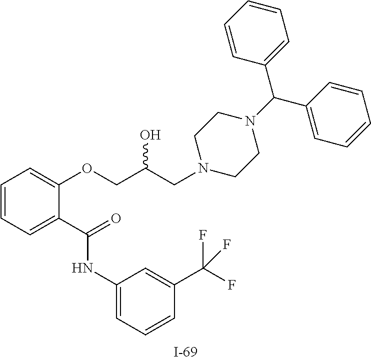 |
| 2-[2-Hydroxy-3-(3-hydroxy-adamantan-1-ylamino)- propoxy]-N-(3-trifluoromethyl-phenyl)-benzamide (I-70) ¹H NMR (DMSO-d₆): δ = 1.31-1.51 (12H), 2.05 (2H), 2.69 (1H), 2.74 (1H), 3.90-3.98 (1H), 4.14-4.25 (2H), 7.11 (1H), 7.22 (1H), 7.43 (1H), 7.51-7.61 (2H), 7.83 (1H), 7.96 (1H), 8.35 (1H), 10.63 (1H); ¹³C NMR (DMSO-d₆): δ = 30.07, 34.92, 40.22, 43.12, 44.29, 49.55, 53.56, 67.56, 68.35, 71.44, 113.52, 115.84, 119.85, 121.00, 123.17, 123.40, 124.14, 129.51, 129.89, 130.57, 133.02, 139.71, 156.28, 164.27 | 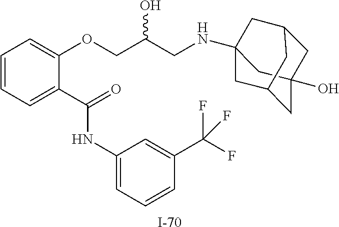 |
| 2-{3-[(Adamantan-1-ylmethyl)-amino]-2-hydroxy- propoxy}-N-naphthalen-2-yl-benzamide (I-71) ¹H NMR: δ = 1.45-1.50 (6H), 1.55-1.63 (3H), 1.66-1.74 (3H), 1.91-1.97 (3H), 2.19-2.34 (2H), 2.79 (1H), 2.86 (1H), 3.57-3.98 (br, 2H), 4.04 (1H), 4.18-4.28 (2H), 6.93 (1H), 7.12 (1H), 7.37 (1H), 7.40-7.46 (2H), 7.75 (1H), 7.76-7.79 (2H), 7.83 (1H), 8.27 (1H), 8.53 (1H), 10.26 (1H); ¹³C NMR: δ = 28.39, 33.43, 37.10, 40.65, 52.07, 62.16, 66.96, 71.09, 112.65, 117.14, 120.89, 121.96, 122.58, 124.83, 126.33, 127.58, 127.96, 128.60, 130.70, 132.57, 133.21, 134.17, 136.48, 156.62, 163.76 | 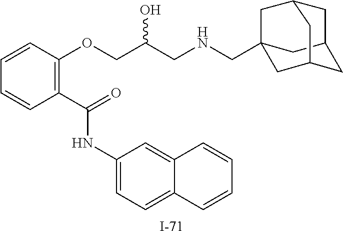 |
| 2-{3-[(Adamantan-1-ylmethyl)-amino]-2-hydroxy- propoxy}-5-chloro-N-(3-trifluoromethyl-phenyl)- benzamide (I-72) ¹H NMR: δ = 1.49-1.53 (6H), 1.60-1.67 (3H), 1.67-1.77 (3H), 1.98 (3H), 2.24-2.33 (2H), 2.73 (1H), 2.84 (1H), 4.01 (1H), 4.09-4.16 (1H), 4.28 (1H), 6.92 (1H), 7.33- 7.47 (3H), 8.07 (1H), 8.11 (1H), 8.22 (1H), 10.33 (1H); ¹³C NMR: δ = 28.49, 33.57, 37.25, 40.80, 51.46, 62.20, 66.86, 71.84, 114.36, 117.40, 120.66, 123.62, C_{q}-1 salicyl not recorded, CF₃ not recorded, 127.54, 129.50, 131.32, 132.39, 133.04, 139.35, 155.25, 162.44 | 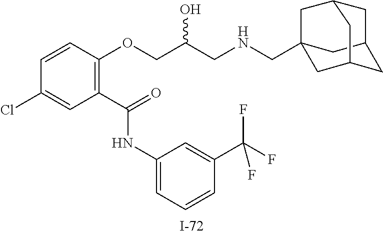 |
| 2-{3-[(Adamantan-1-ylmethyl)-amino]-2-hydroxy- propoxy}-N-(2-allyl-phenyl)-benzamide (I-73) ¹H NMR: δ = 1.40-1.48 (6H), 1.54-1.62 (3H), 1.65-1.73 (3H), 1.89-1.98 (3H), 2.20-2.43 (2H), 2.76-2.88 (1H), 2.92- 3.02 (1H), 3.40-3.52 (2H), 4.16 (1H), 4.22 (1H), 4.24-4.30 (1H), 4.99-5.10 (2H), 5.90-6.03 (1H), 7.02 (1H), 7.12 (1H), 7.09-7.19 (1H), 7.46 (1H), 7.78 (1H), 8.15 (1H), 9.37 (1H); ¹³C NMR: δ = 28.20, 33.09, 36.11, 36.83, 40.22, 52.45, 61.67, 66.02, 71.56, 113.34, 116.61, 122.09, 123.17, 125.49, 125.93, 127.11, 129.89, 132.37, 133.06, 133.09, 136.00, 136.32, 156.68, 164.30 | 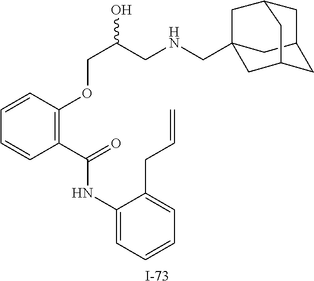 |
| 2-{3-[(Adamantan-1-ylmethyl)-amino]-2-hydroxy- propoxy}-N-(4-bromo-phenyl)-benzamide (I-74) ¹H NMR: δ = 1.51 (6H), 1.64 (3H), 1.74 (3H), 1.99 (3 H), 2.23 (1H), 2.34 (1H), 2.74 (1H), 2.86 (1H), 4.04 (1H), 4.15 (1H), 4.27 (1H), 6.95 (1H), 7.12 (1H), 7.43 (2H), 7.45 (1H), 7.74 (2H), 8.23 (1H), 10.17 (H); ¹³C NMR: δ = 28.47, 33.60, 37.21, 40.83, 51.93, 62.42, 67.17, 71.21, 112.73, 116.45, 122.04, 122.17, 122.38, 131.91, 132.65, 133.34, 138.18, 156.64, 163.59 | 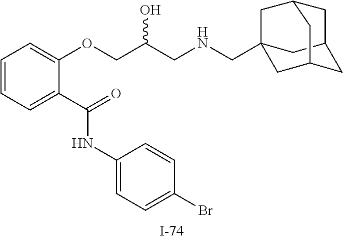 |
| 2-[3-(Adamantan-1-ylamino)-2-hydroxy-propoxy]-N- (4-bromo-phenyl)-benzamide (I-75) ¹H NMR: δ = 1.52-1.69 (12H), 2.04 (3H), 2.72 (1H), 2.90 (1H), 4.02-4.28 (3H), 6.95 (1H), 7.12 (1H), 7.40- 7.49 (3H), 7.71-7.76 (2H), 8.21 (1H), 10.13 (1H); ¹³C NMR: δ = 29.53, 36.49, 42.35, 42.42, 51.72, 67.89, 71.29, 112.67, 116.47, 122.02, 122.31, 122.43, 131.87, 132.60, 133.31, 138.13, 156.61, 163.64 | 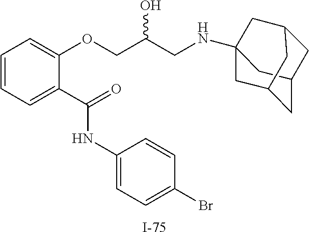 |
| N-(4-Bromo-phenyl)-2-[2-hydroxy-3-(3-hydroxy- adamantan-1-ylamino)-propoxy]-benzamide (I-76) ¹H NMR: δ = 1.33-1.49 (12H), 2.06 (2H), 2.61-2.70 (2H), 3.84-3.92 (1H), 4.12 (1H), 4.22 (1H), 7.10 (1H), 7.21 (1H), 7.48-7.56 (3H), 7.77 (2H), 7.83 (1H), 10.40; ¹³C NMR: δ = 30.20, 35.10, 40.83, 43.28, 44.42, 50.11, 53.07, 67.73, 68.94, 71.60, 113.56, 115.23, 121.04, 121.79, 123.21, 130.66, 131.56, 133.01, 138.34, 156.33, 163.84 | 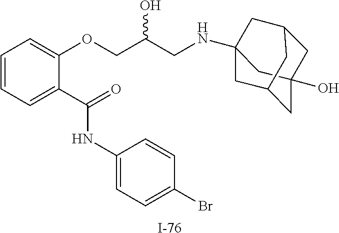 |
| N-(2-Fluoro-phenyl)-2-[2-hydroxy-3-(4-phenyl- piperidin-1-yl)-propoxy]-benzamide (I-77) ¹H NMR: δ = 1.72-1.92 (4H), 2.14 (1H), 2.43 (1H), 2.48-2.67 (3H), 2.85-2.92 (1H), 3.13-3.20 (1H), 4.22- 4.32 (3H), 7.03-7.25 (8H), 7.28-7.34 (2H), 7.50 (1H), 8.28 (1H), 8.53 (1H), 10.10 (1H); ¹³C NMR: δ = 33.45, 33.80, 42.48, 52.91, 56.17, 60.75, 65.21, 72.16, 113.00, 114.87, 121.99, 122.08, 122.90, 124.32, 124.74, 126.41, 126.93, 127.12, 128.61, 132.75, 133.58, 146.08, 153.14, 156.93, 163.60 | 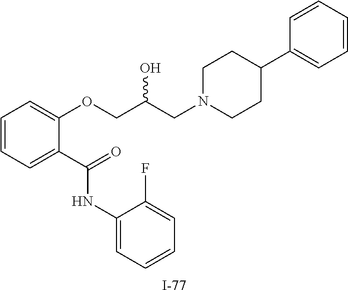 |
| 2-[3-(4-Benzyl-piperidin-1-yl)-2-hydroxy-propoxy]-N- (2-fluoro-phenyl)-benzamide (I-78) ¹H NMR: δ = 1.22-1.36 (2H), 1.48-1.59 (1H), 1.59-1.69 (2H), 1.93 (1H), 2.23 (1H), 2.47-2.57 (4H), 2.69-2.76 (1H), 2.96-3.03 (1H), 4.16-4.26 (3H), 7.02-7.22 (8H), 7.24-7.32 (2H), 7.48 (1H), 8.28 (1H), 8.51 (1H), 10.09 (1H); ¹³C NMR: δ = 32.30, 32.60, 37.88, 43.26, 52.52, 55.64, 60.66, 65.14, 72.22, 112.98, 114.87, 121.95, 122.04, 122.89, 124.29, 124.71, 126.01, 127.12, 128.35, 129.24, 132.75, 133.55, 140.66, 153.14, 156.95, 163.58 | 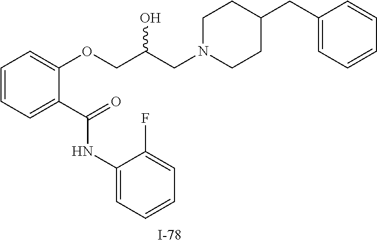 |
| 2-[3-(4-Benzhydryl-piperazin-1-yl)-2-hydroxy- propoxy]-N-(2-fluoro-phenyl)-benzamide (I-79) ¹H NMR: δ = 2.34-2.50 (6H), 2.51-2.63 (2H), 2.65- 2.78 (2H), 4.17-4.27 (4H), 7.00-7.22 (7H), 7.23-7.32 (4H), 7.37-7.43 (4H), 7.48 (1H), 8.27 (1H), 8.51 (1H), 10.08 (1H); ¹³C NMR: δ = 52.06, 53.50, 60.35, 65.11/65.12, 72.15, 76.31, 112.97, 114.87, 121.99, 122.04, 122.87, 124.29, 124.72, C_{q}-1′ in 2′-F-aniline not recorded, 127.13, 128.05, 128.65, 132.76, 133.56, 142.73, 153.10, 156.91, 163.56 | 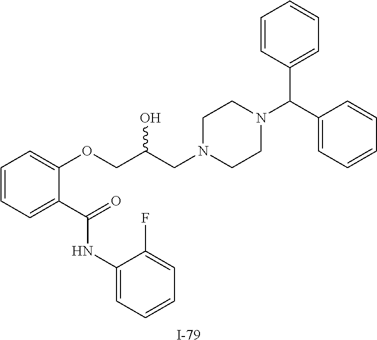 |
| 2-[2-Hydroxy-3-(4-phenyl-piperidin-1-yl)-propoxy]-N- p-tolyl-benzamide (I-80) ¹H NMR: δ = 1.71-193 (4H), 2.14 (1H), 2.32 (3H), 2.44 (1H), 2.48-2.58 (2H), 2.70 (1H), 2.79-2.86 (1H), 3.12-3.19 (1H), 4.08 (1H), 4.19-4.26 (1H), 4.33 (1H), 6.98 (1H), 7.10-7.18 (3H), 7.20-7.25 (3H), 7.29-7.36 (2H), 7.46 (1H), 7.66-7.72 (2H), 8.26 (1H), 10.02 (1H); ¹³C NMR: δ = 21.04, 33.45, 33.80, 42.43, 52.85, 56.30, 60.17, 65.25, 70.94, 112.63, 120.55, 121.97, 122.84, 126.47, 126.92, 128.65, 129.48, 132.67, 133.02, 133.57, 136.47, 145.97, 156.62, 163.39 | 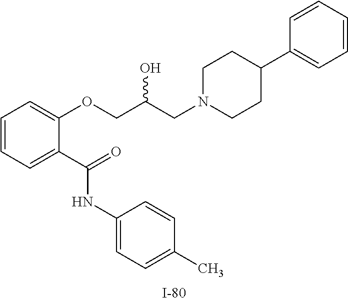 |
| 2-[3-(4-Benzyl-piperidin-1-yl)-2-hydroxy-propoxy]-N- p-tolyl-benzamide (I-81) ¹H NMR: δ = 1.25-1.37 (2H), 1.49-1.70 (3H), 1.96 (1H), 2.24 (1H), 2.33 (3H), 2.43 (1H), 2.55 (2H), 2.62 (1H), 2.66-2.72 (1H), 2.96-3.04 (1H), 4.03 (1H), 4.14- 4.22 (1H), 4.28 (1H), 6.95 (1H), 7.08-7.17 (5H), 7.18- 7.23 (1H), 7.27-7.32 (2H), 7.43 (1H), 7.64-7.70 (2H), 8.24 (1H), 10.00 (1H); ¹³C NMR: δ = 21.03, 32.09, 32.39, 37.74, 43.15, 52.53, 55.69, 60.12, 65.14, 70.96, 112.61, 120.51, 121.90, 122.79, 126.06, 128.36, 129.21, 129.45, 132.59, 132.98, 133.49, 136.44, 140.49, 156.58, 163.37 | 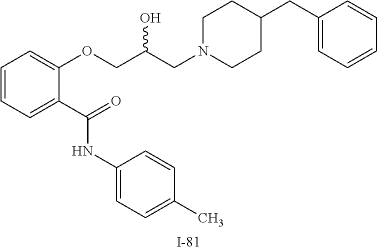 |
| 2-[3-(4-Benzhydryl-piperazin-1-yl)-2-hydroxy- propoxy]-N-p-tolyl-benzamide (I-82) 00¹H NMR: δ = 2.29 (3H), 2.34-2.53 (6H), 2.46 (1H), 2.61-2.76 (2H), 2.65 (1H), 4.03 (1H), 4.12-4.19 (1H), 4.25 (1H), 4.29 (1H), 6.95 (1H), 7.07-7.14 (3H), 7.16- 7.22 (2H), 7.25-7.31 (4H), 7.38-7.47 (5H), 7.62-7.67 (2H), 8.24 (1H), 9.99 (1H); ¹³C NMR: δ = 20.85, 51.86, 53.40, 59.63, 64.96, 70.73, 76.09, 112.46, 120.33, 121.79, 122.64, 127.01, 127.89, 128.50, 129.30, 132.48, 132.83, 133.33, 136.26, 142.46, 142.52, 156.42, 163.18 | 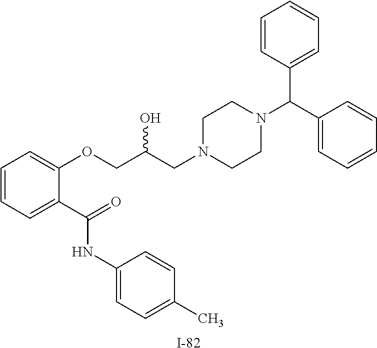 |
| 5-Chloro-2-[2-hydroxy-3-(4-phenyl-piperidin-1-yl)- propoxy]-N-(3-trifluoromethyl-phenyl)-benzamide (I-83) ¹H NMR (DMSO-d₆): δ = 1.55-1.72 (4H), 1.96-2.09 (2H), 2.35-2.56 (2H), 2.83-2.91 (1H), 2.92-3.01 (1H), 4.03-4.11 (1H), 4.12-4.27 (2H), 5.19 (1H). 7.14-7.23 (3H), 7.24-7.34 (3H), 7.46 (1H), 7.56 7.64 (2H), 7.79 (1H), 7.99 (1H), 8.29 (1H), 10.62 (1H); ¹³C NMR (DMSO-d₆): δ = 33.08, 41.67, 54.46, 61.13, 66.38, 72.32, 115.81, 115.90, 120.13, 123.41, 124.58, 124.82, 125.10, 125.93, 126.59, 128.28, 129.51, 129.61, 129.99, 132.34, 139.45, 146.24, 155.18, 162.93 | 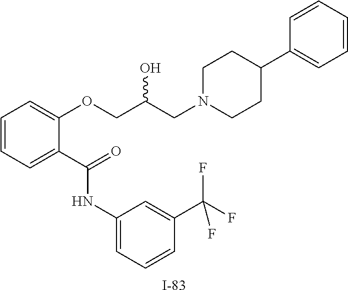 |
| 2-[3-(4-Benzyl-piperidin-1-yl)-2-hydroxy-propoxy]-5- chloro-N-(3-trifluoromethyl-phenyl)-benzamide (I-84) ¹H NMR: δ = 1.24-1.37 (2H), 1.49-1.72 (3H), 1.97 (1H), 2.29 (1H), 2.43 (1H), 2.52-2.60 (3H), 2.70 (1H), 3.01 (1H), 4.00 (1H), 4.16-4.24 (1H), 4.29 (1H), 6.91 (1H), 7.12-7.16 (2H), 7.17-7.22 (1H), 7.24-7.32 (2H), 7.37 (1H), 7.42 (1H), 7.46 (1H), 8.08 (2H), 8.22 (1H), 10.28 (1H); ¹³C NMR: δ = 32.13, 32.40, 37.80, 43.15, 52.49, 55.82, 59.78, 65.03, 71.80, 114.32, 117.35, 120.68, 123.55, 123.69, 124.21, 126.07, 127.58, 128.39, 129.23, 129.55, 131.31, 132.39, 133.06, 139.31, 140.51, 155.19, 162.43 | 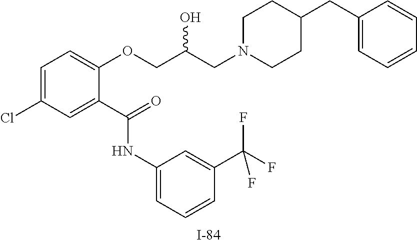 |
| 2-[3-(4-Benzhydryl-piperazin-1-yl)-2-hydroxy- propoxy]-5-chloro-N-(3-trifluoromethyl-phenyl)- benzamide (I-85) ¹H NMR: δ = 2.37-2.52 (7H), 2.59 (1H), 2.68-2.78 (2H), 3.96-4.02 (1H), 4.15-4.22 (1H), 4.26 (1H), 4.29 (1H), 6.90 (1H), 7.16-7.22 (2H), 7.24-7.31 (5H), 7.32- 7.36 (1H), 7.39-7.44 (5H), 8.01-8.07 (2H), 8.21 (1H), 10.26 (1H); ¹³C NMR: δ = 51.93, 53.62 (br), 59.56, 64.96, 71.69, 76.18, 114.30, 117.29, 120,66, 123.48, 123.62, 124.15, 127.17, 127.59, 128.04, 128.66, 129.52, 131.29, 132.39, 133.06, 139.27), 142.64, 155.14, 162.37 | 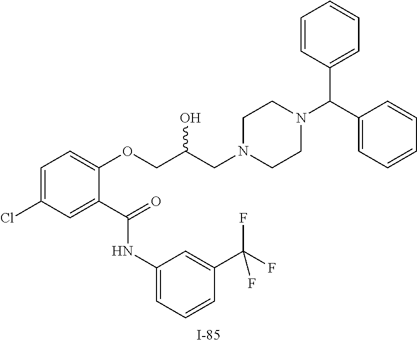 |
| 3-{3-[(Adamantan-1-ylmethyl)-amino]-2-hydroxy- propoxy}-naphthalene-2-carboxylic acid (4-fluoro- phenyl)-amide (I-86) ¹H NMR: δ = 1.51 (6H), 1.60-1.67 (3H), 1.70-1.77 (3H), 1.98 (3H), 2.26 (1H), 2.32 (1H), 2.79 (1H), 2.86 (1H), 4.07-4.13 (1H), 4.14-4.21 (2H), 7.06 (2H), 7.59 (2H), 7.73 (1H), 7.84 (2H), 7.89 (1H), 8.20 (1H), 8.24 (1H), 10.16 (1H); ¹³C NMR: δ = 28.51, 33.63, 37.28, 40.90, 51.64, 62.45, 68.06, 78.53, 115.54, 122.39, 122.60, 122.99, 125.07, 126.89, 127.08, 127.60, 128.23, 128.46, 134.83, 136.97, 153.90, 159.49, 163.51 | 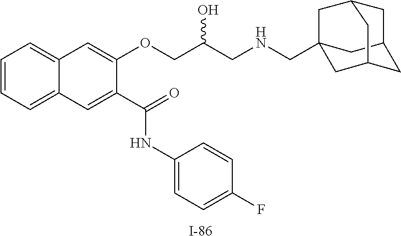 |
| 3-[3-(Adamantan-1-ylamino)-2-hydroxy-propoxy]- naphthalene-2-carboxylic acid (4-fluoro-phenyl)-amide (I-87) ¹H NMR: δ = 1.55-1.81 (12H), 2.10 (3H), 2.89 (1H), 3.02 (1H), 4.12 (1H), 4.17 (1H), 4.35 (1H), 7.00-7.07 (2H), 7.51-7.59 (2H), 7.71 (1H), 7.78-7.88 (3H), 8.14- 8.20 (2H), 9.99 (1H); ¹³C NMR: δ = 29.41, 36.24, 41.42, 42.69, 53.52, 68.11, 78.21, 115.58, 122.53, 122.72, 122.89, 125.13, 126.89, 126.99, 127.49, 128.24, 128.45, 134.69, 136.88, 153.63, 159.53, 163.67 | 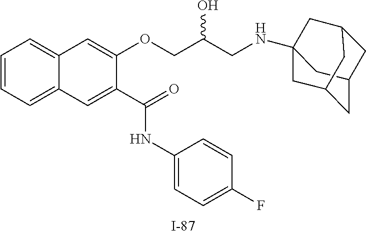 |
| 5-Bromo-N-(4-fluoro-phenyl)-2-[2-hydroxy-3-(4- phenyl-piperidin-1-yl)-propoxy]-benzamide (I-88) ¹H NMR: δ = 1.71-1.94 (4H), 2.16 (1H), 2.42-2.59 (3H), 2.84 (1H), 3.14 (1H), 4.02 (1H), 4.18-4.25 (1H), 4.31 (1H), 6.86 (1H), 7.00-7.07 (2H), 7.20-7.25 (3H), 7.30-7.36 (2H), 7.54 (1H), 7.73-7.79 (2H), 8.36 (1H), 10.07 (1H); ¹³C NMR: δ = 33.40, 33.74, 42.35, 52.94, 56.31, 59.99, 65.16, 71.51, 114.65, 114.68, 115.57, 122.20, 124.29, 126.51, 126.88, 128.66, 134.79, 135.24, 135.73, 145.79, 155.62, 159.41, 162.01 | 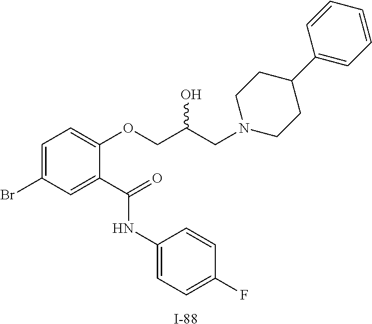 |
| 2-[3-(4-Benzyl-piperidin-1-yl)-2-hydroxy-propoxy]-5- bromo-N-(4-fluoro-phenyl)-benzamide (I-89) ¹H NMR: δ = 1.23-1.37 (2H), 1.50-1.60 (1H), 1.61-1.71 (2H), 1.97 (1H), 2.27 (1H), 2.42 (1H), 2.53 2.61 (3H), 2.70 (1H), 2.99 (1H), 3.97 (1H), 4.13-4.20 (1H), 4.26 (1H), 6.83 (1H), 6.99 7.06 (2H), 7.11-7.16 (2H), 7.18- 7.23 (1H), 7.25-7.32 (2H), 7.52 (1H), 7.71-7.77 (2H), 8.34 (1H), 10.04 (1H); ¹³C NMR: δ = 32.10, 32.43, 37.76, 43.13, 52.57, 55.78, 59.89, 65.04, 71.49, 114.63, 114.65, 115.56, 122.19, 124.29, 126.09, 128.39, 129.23, 134.78, 135.23, 135.70, 140.42, 155.61, 159.41, 162.00 | 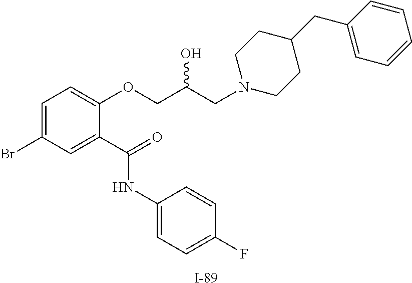 |
| 2-[3-(4-Benzhydryl-piperazin-1-yl)-2-hydroxy- propoxy]-5-bromo-N-(4-fluoro-phenyl)-benzamide (I-90) ¹H NMR: δ = 2.35-2.52 (7H), 2.61 (1H), 2.67-2.78 (2H), 3.98 (1H), 4.12-4.20 (1H), 4.24-4.30 (2H), 6.84 (1H), 6.96-7.03 (2H), 7.16-7.22 (2H), 7.25-7.31 (4H), 7.38-7.44 (4H), 7.53 (1H), 7.68-7.74 (2H), 8.35 (1H), 10.02 (1H); ¹³C NMR: δ = 51.92, 53.55 (dyn), 59.63, 64.99, 71.43, 76.18, 114.65, 114.69, 115.57, 122.14, 124.28, 127.20, 128.05, 128.68, 134.75, 135.27, 135.72, 142.47, 142.54, 155.59, 159.38, 161.97 | 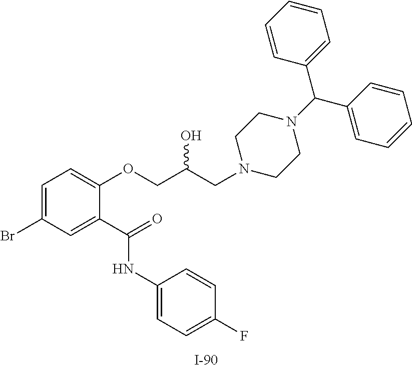 |
| 2-{3-[(Adamantan-1-ylmethyl)-amino]-2-hydroxy- propoxy}-5-bromo-N-(4-fluoro-phenyl)-benzamide (I-91) ¹H NMR: δ = 1.48-1.54 (6H), 1.60-1.67 (3H), 1.70- 1.77 (3H), 1.98 (3H), 2.22-2.32 (2H), 2.71 (1H), 2.83 (1H), 4.00 (1H), 4.05-4.11 (1H), 4.24 (1H), 6.84 (1H), 6.97-7.04 (2H), 7.51 (1H), 7.72-7.78 (2H), 8.34 (1H), 10.09 (1H); ¹³C NMR: δ = 28.49, 33.64, 37.26, 40.89, 51.64, 62.44, 67.18, 71.73, 114.58, 114.70, 115.54, 122.21, 124.22, 134.83, 135.19, 135.70, 155.70, 159.40, 162.02 | 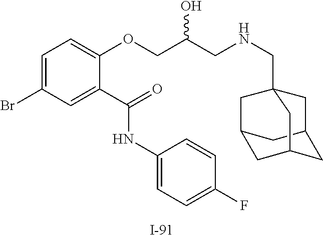 |
| 2-[3-(Adamantan-1-ylamino)-2-hydroxy-propoxy]-5- bromo-N-(4-fluoro-phenyl)-benzamide (I-92) ¹H NMR (DMSO-d6): δ = 1.41-1.52 (9H), 1.54-1.62 (3H), 1.94 (3H), 2.59-2.70 (2H), 3.83-3.89 (1H), 4.12 (1H), 4.21 (1H), 7.14-7.23 (3H), 7.68 (1H), 7.76-7.82 (2H), 7.89 (1H), 10.34 (1H); ¹³C NMR (DMSO-d6): δ = 28.88, 36.21, 41.97, 42.79, 49.79, 68.86, 69.77, 71.94, 112.31, 115.26, 116.07, 121.61, 125.59, 132.50, 134.99, 135.07, 155.53, 158.31, 162.14 | 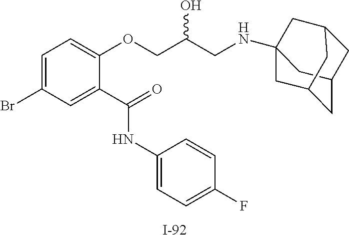 |

The compound of formula I-46 in TABLE 1 was synthesized in the form of the highly enriched (R)- and in the form of the highly enriched (S)-enantiomer, respectively.

The compounds of formula INT-14 and INT-15 in TABLE 1, both are not compounds of formula I, but are novel intermediates in the preparation of a compound of formula I and also form part of the present invention.

Further chemical characterization data of novel intermediates

### 5-Chloro-2-hydroxy-N-naphthalen-1-ylmethyl-benzamide

¹H NMR: δ=5.08 (2H), 6.38 (1H), 6.95 (1H), 7.20 (1H), 7.31 (1H), 7.45-7.60 4H), 7.88 (1H), 7.92 (1H), 8.02 (1H), 12.25 (1H); ¹³C NMR: δ=42.26, 115.11, 120.36, 123.27, 123.49, 125.10, 125.61, 126.43, 127.23, 127.44, 129.17, 129.43, 131.54, 132.32, 134.16, 134.38, 160.40, 168.67

### N-(4-Fluoro-phenyl)-2-hydroxy-5-prop-2-ynyloxy-benzamide

¹H NMR (DMSO-d₆): δ=3.59 (1H), 4.83 (2H), 6.88 (1H), 7.06-7.10 (2H), 7.14-7.21 (2H), 7.69-7.77 (2H), 9.36 (1H), 10.14 (1H); ¹³C NMR: δ=57.41, 78.52, 79.21, 115.24, 115.80, 115.96, 118.18, 121.39, 126.08, 135.31, 147.37, 151.79, 158.15, 163.78

### N-(2-Methoxy-benzyl)-2-oxiranylmethoxy-benzamide

Useful for the preparation of compounds of formula I-2, I-7, I-18, and I-29 according to REACTION SCHEME 1. ¹H NMR: δ=2.64 (1H), 2.77 (1H), 3.22-3.26 (1H), 3.87 (3H), 4.07 (1H), 4.31 (1H), 4.68 (2H), 6.88 (1H), 6.91 (1H), 6.92 (1H), 7.09 (1H), 7.25 (1H), 7.38 (1H, 7.40 (1H), 8.15 (1H), 8.21 (1H); ¹³C NMR: δ=39.46, 44.43, 49.69, 55.51, 69.61, 110.45, 112.86, 120.75, 122.10, 122.69, 126.86, 128.77, 129.83, 132.64 (2×), 156.42, 157.76, 165.00

### N-(4-Methoxy-benzyl)-2-oxiranylmethoxy-benzamide

Useful for the preparation of compounds of formula I-8 and I-51 according to REACTION SCHEME 1.

¹H NMR: δ=2.62 (1H), 2.75 (1H), 3.22-3.26 (1H), 3.79 (3H), 4.10 (1H), 4.31 (1H), 4.54-4.65 (2H), 6.86, 6.88 (2H), 6.91 (1H), 7.11 (1H), 7.32, 7.34 (2H), 7.41 (1H), 8.11 (1H), 8.22 (1H); ¹³C NMR: δ=43.59, 44.50, 49.69, 55.46, 69.04, 112.87, 114.15, 122.22, 122.47, 129.45, 131.02, 132.65, 132.78, 156.37, (C_{q}-4 in 4-methoxybenzyl not recorded), 164.98

### 2-Oxiranylmethoxy-N-phenethyl-benzamide

Useful for the preparation of compounds of formula I-9 and I-10 and I-31 according to REACTION SCHEME 1. ¹H NMR: δ=2.66-2.68 (1H), 2.83-2.86 (1H), 2.99-3.03 (2H), 3.12-3.15 (1H), 3.75-3.87 (2H), 4.03 (1H), 4.31 (1H), 6.96 (1H), 7.14 (1H), 7.23-7.38 (5H), 7.44 (1H), 7.98 (1H), 8.25 (1H); ¹³C NMR: δ=35.64, 41.09, 44.43, 49.66, 69.21, 113.06, 122.13, 122.39, 126.40, 128.61, 128.97, 132.46, 132.69, 139.52, 156.40, 165.16

### 5-Chloro-2-oxiranylmethoxy-N-phenethyl-benzamide

Useful for the preparation of a compound of formula I-33 according to REACTION SCHEME 1. ¹H NMR: δ=2.60-2.62 (1H), 2.80-2.83 (1H), 2.95-2.98 (2H), 3.07-3.10 (1H), 3.70-3.82 (2H), 3.95 (1H), 4.27 (1H), 6.87 (1H), 7.20-7.33 (5H), 7.35 (1H), 7.87 (1H), 8.17 (1H); ¹³C NMR: δ=35.54, 41.18, 44.39, 49.57, 69.73, 114.71, 123.90, 126.50, 127.64, 128.67, 128.98, 132.21, 132.30, 139.36, 154.93, 163.87

### 2-(Oxiran-2-ylmethoxy)-N-(3-phenylpropyl)benzamide

Useful for the preparation of compounds of formula I-11 and I-32 according to REACTION SCHEME 1. ¹H NMR: δ=1.94-2.01 (2H), 2.71-2.75 (2H), 2.80-2.82 (1H), 2.92-2.94 (1H), 3.37-3.41 (1H), 3.46-3.58 (2H), 4.08 (1H), 4.45 (1H), 6.94 (1H), 7.10 (1H), 7.15-7.30 (5H), 7.42 (1H), 7.91 (1H), 8.20 (1H); ¹³C NMR: δ=31.28, 33.54, 39.67, 44.56, 49.85, 69.19, 112.89, 122.21, 122.61, 126.00, 128.52, 128.56, 132.58, 132.66, 141.83, 156.32, 165.23

### N-(3-Methyl-butyl)-2-oxiranylmethoxy-benzamide

Useful for the preparation of compounds of formula I-24, I-25, I-56, I-57, I-58, I-59 and I-60 according to REACTION SCHEME 1. ¹H NMR: δ=0.94 (6H), 1.49-1.55 (2H), 1.65-1.75 (1H), 2.79-2.81 (1H), 2.92-2.95 (1H), 3.35-3.39 (1H), 3.41-3.54 (2H), 4.06 (1H), 4.41 (1H), 6.92 (1H), 7.08 (1H), 7.39 (1H), 7.81 (1H), 8.18 (1H); ¹³C NMR: δ=22.59, 26.07, 38.26, 38.42, 44.52, 49.79, 69.19, 112.79, 122.12, 122.61, 132.49, 132.55, 156.29, 165.07

### 2-Oxiranylmethoxy-N-(4-trifluoromethyl-benzyl)-benzamide

Useful for the preparation of compounds of formula I-16 and I-30 according to REACTION SCHEME 1. ¹H NMR: δ=2.66-2.68 (1H), 2.79-2.81 (1H), 3.26-3.29 (1H), 4.09 (1H), 4.42 (1H), 4.65-4.80 (2H), 6.94 (1H), 7.12 (1H), 7.44 (1H), 7.50, 7.52 (2H), 7.58-7.60 (2H), 8.21 (1H), 8.32 (1H); ¹³C NMR: δ=43.55, 44.39, 49.74, 68.92, 113.05, 122.11, 122.36, 123.5 (HMBC), 125.68, 128.20, 132.70, 133.07, 143.08, 156.41, 165.34

### 5-Chloro-N-naphthalen-1-ylmethyl-2-oxiranylmethoxy-benzamide

Useful for the preparation of compounds of formula I-34 and I-35 according to REACTION SCHEME 1. ¹H NMR: δ=2.25-2.27 (1H), 2.35-2.37 (1H), 2.81-2.84 (1H), 3.96 (1H), 4.07 (1H), 5.04-5.17 (2H), 6.79 (1H), 7.33 (1H), 7.43-7.58 (4H), 7.82 (1H), 7.88 (1H), 8.09 (1H), 8.12 (1H), 8.22 (1H); ¹³C NMR: δ=42.34, 44.19, 49.14, 69.33, 114.35, 123.67, 123.86, 125.65, 126.07, 126.70, 127.12, 127.64, 128.67, 128.89, 131.69, 132.36, 132.44, 133.84, 134.07, 154.85, 163.54

### N-(4-Fluoro-phenyl)-2-oxiranylmethoxy-benzamide

Useful for the preparation of compounds of formula I-3, I-4, I-5, I-37, I-46, I-47, I-48, I-55 and I-61 according to REACTION SCHEME 1. ¹H NMR: δ=2.88-2.90 (1H), 2.98-3.01 (1H), 3.47-3.51 (1H), 4.13 (1H), 4.59 (1H), 6.98 (1H), 7.01-7.08 (2H), 7.15 (1H), 7.47 (1H), 7.75-7.80 (2H), 8.27 (1H), 9.81 (1H); ¹³C NMR: δ=44.66, 49.96, 68.91, 112.78, 115.68, 121.94, 122.47, 122.50, 132.86, 133.33, 134.87, 156.02, 159.36, 163.12

### N-(2-Fluoro-phenyl)-2-oxiranylmethoxy-benzamide

Useful for the preparation of a compound of formula I-38 according to REACTION SCHEME 1. ¹H NMR: δ=2.76-2.78 (1H), 2.99-3.01 (1H), 3.51-3.55 (1H), 4.18 (1H), 4.40 (1H), 7.02 (1H), 7.04-7.20 (4H), 7.49 (1H), 8.31 (1H), 8.60 (1H), 10.21 (1H); ¹³C NMR: δ=45.07, 49.53, 71.11, 112.69, 114.79, 121.97, 122.14, 122.34, 124.08, 124.80, 127.29, 132.92, 133.65, 152.89, 156.43, 163.17

### N-(2,4-Difluoro-phenyl)-2-oxiranylmethoxy-benzamide

Useful for the preparation of a compound of formula I-39 according to REACTION SCHEME 1. ¹H NMR: δ=2.76-2.28 (1H), 2.99-3.01 (1H), 3.49-3.53 (1H), 4.15 (1H), 4.43 (1H), 6.86-6.94 (2H), 7.01 (1H), 7.16 (1H), 7.50 (1H), 8.30 (1H), 8.52-8.58 (1H), 10.11 (1H); ¹³C NMR: δ=44.96, 49.52, 71.04, 103.57, 111.31, 112.70, 121.72, 122.38, 123.04, 123.62, 132.89, 133.74, 152.84, 156.41, 158.47, 163.15

### 2-Oxiranylmethoxy-N-(3-trifluoromethyl-phenyl)-benzamide

Useful for the preparation of a compound of formula I-42 according to REACTION SCHEME 1. ¹H NMR: δ=2.90-2.92 (1H), 3.00-3.03 (1H), 3.49-3.53 (1H), 4.16 (1H), 4.61 (1H), 7.00 (1H), 7.17 (1H), 7.36 (1H), 7.46 (1H), 7.49 (1H), 7.95 (1H), 8.22 (1H), 8.27 (1H), 10.00 (1H); ¹³C NMR: δ=44.69, 49.94, 68.76, 112.85, 117.17, 120.64, 122.18, 122.59, 123.31, 123.86, 129.57, 131.50, 132.93, 133.64, 139.34, 156.06, 163.46

### N-(4-Fluoro-phenyl)-2-oxiranylmethoxy-5-prop-2-ynyloxy-benzamide

Useful for the preparation of a compound of formula I-45 according to REACTION SCHEME 1. ¹H NMR: δ=2.66 (1H), 2.74-2.77 (1H), 2.89-2.92 (1H), 3.33-3.38 (1H), 3.96 (1H), 4.31 (1H), 4.84 (2H), 6.99-7.07 (3H), 7.09 (1H), 7.63-7.69 (2H), 7.82 (1H), 9.85 (1H); ¹³C NMR: δ=44.69, 50.22, 58.41, 69.55, 77.22, 77.35, 115.39, 115.74, 116.68, 120.80, 122.04, 123.42, 134.56, 150.03, 153.98, 159.48, 162.55

### N-(2-Allyl-phenyl)-2-oxiranylmethoxy-benzamide

Useful for the preparation of compounds of formula I-65 according to REACTION SCHEME 1. ¹H NMR: δ=2.75 (1H), 2.92 (1H), 3.37-3.42 (1H), 3.48 (2H), 4.13 (1H), 4.54 (1H), 5.02 (1H), 5.10 (1H), 5.95-6.09 (1H), 7.06 (1H), 7.12-7.19 (2H), 7.23 (1H), 7.27-7.32 (1H), 7.44-7.52 (1H), 8.00 (1H), 8.28 (1H), 9.41 (1H); ¹³C NMR: δ=36.00, 44.81, 49.81, 70.46, 113.04, 116.60, 122.36, 122.76, 124.67, 125.50, 127.24, 129.89, 131.57, 132.97, 133.23, 136.01, 136.14, 156.33, 163.66

### 5-Chloro-N-(4-fluoro-phenyl)-2-oxiranylmethoxy-benzamide

Useful for the preparation of compounds of formula I-66 according to REACTION SCHEME 1. ¹H NMR: δ=2.88 (1H), 3.00 (1H), 3.46-3.51 (1H), 4.08 (1H), 4.58 (1H), 6.92 (1H), 7.00-7.08 (2H), 7.40 (1H), 7.70-7.78 (2H), 8.22 (1H), 9.74 (1H); ¹³C NMR: δ=44.67, 49.80, 69.39, 114.34, 115.75, 122.00, 123.86, 127.96, 132.48, 132.90, 134.52, 154.49, 159.50, 161.75

### 5-Chloro-2-oxiranylmethoxy-N-(3-trifluoromethyl-phenyl)-benzamide

Useful for the preparation of compounds of formula I-83, I-84, I-85 according to REACTION SCHEME 1. ¹H NMR: δ=2.90 (1H), 3.02 (1H), 3.47-3.53 (1H), 4.12 (1H), 4.60 (1H), 6.94 (1H), 7.37 (1H), 7.42 (1H), 7.46 (1H), 7.92 (1H), 8.19 (1H), 8.22 (1H), 9.91 (1H); ¹³C NMR: δ=44.69, 49.78, 69.25, 114.42, 117.18, 120.92, 123.36, 124.12, 123.55, 128.05, 129.63, 131.50, 132.54, 133.20, 139.00, 154.53, 162.08

### N-Naphthalen-2-yl-2-oxiranylmethoxy-benzamide

Useful for the preparation of compounds of formula I-71 according to REACTION SCHEME 1. ¹H NMR: δ=2.92 (1H), 3.02 (1H), 3.51-3.56 (1H), 4.17 (1H), 4.61 (1H), 7.01 (1H), 7.15-7.21 (1H), 7.37-7.42 (1H), 7.43-7.48 (1H), 7.48-7.52 (1H), 7.69 (1H), 7.78 (1H), 7.83 (1H), 7.85 (1H), 8.33 (1H), 8.59 (1H), 9.99 (1H); ¹³C NMR: δ=44.71, 49.99, 69.09, 112.75, 117.07, 120.57, 122.51, 122.67, 124.93, 126.46, 127.64, 128.04, 128.80, 130.79, 132.91, 133.33, 134.24, 136.21, 156.08, 163.38

### N-(2-Allyl-phenyl)-2-hydroxy-benzamide

¹H NMR: δ=3.47 (2H), 5.15 (1H), 5.29 (1H), 6.01-6.13 (1H), 6.88-6.93 (1H), 7.04 (1H), 7.18-7.28 (2H), 7.32-7.37 (1H), 7.41-7.48 (2H), 7.86 (1H), 8.10 (1H), 12.11 (1H); ¹³C NMR: δ=37.23, 114.68, 117.16, 119.07, 119.12, 124.74, 125.41, 126.41, 127.76, 130.75, 131.30, 134.79, 135.12, 136.47, 162.17

### REFERENCES

1) WHO Malaria Report 2011: www.who.int/malaria/world_malaria_report_2011
2) Noedl H, Se Y, Schaechner K, Smith B L, Socheat D, Fukuda M. Evidence of artemisinin-resistant malaria in western Cambodia. N. Engl. J. Med. 2008; 359, 2619-20.
3) Cheeseman I A, et. al. A Major Genome Region Underlying Artemisinin Resistance in Malaria. Science 2012; 336, 79-82.
4) Phyo A P, et. al. Emergence of artemisinin-resistant malaria on the western border of

Thailand: a longitudinal study. The Lancet 5 Apr. 2012 (in press DOI: 10.1016/50140-6736(12)60484-X).
5) Hüttinger F, Satimai W, Wernsdorfer G, Wiedermann U, Congpuong K, Wernsdorfer W H. Sensitivity to artemisinin, mefloquine and quinine of Plasmodium falciparum in northwestern Thailand. Wien. Klin Wochenschr. 2010; 122 [S3], 52-56.
6) Weisman J L, Liou A P, Shelat A A, Cohen F E, Kiplin G R, and DeRisi J L. Searching for New Antimalarial Therapeutics amongst Known Drugs. Chem. Biol. Drug Des. 2006; 67, 409-16.
7) Tasanor O, Ernst M, Thriemer K, Ecker G F, Noedl H, Chiba P. Characterization of a novel class of antimalarials and its applicability to plasmodial target identification. Wien. Klin. Wochenschr. 2007; 119(S3), 83-87.
8) Lowes D J, et. al. Optimization of Propafenone Analogues as Antimalarial Leads. J. Med. Chem. 2011; 54 (21), 7477-7485.
9) Brana M F, Lopez Rodriguez M L, Moran M. Synthesis of aza analogs of propafenone. An. Quim. 1990; 86(5), 565-8.
10) Trager W, Jensen J B. Human malaria parasites in continuous culture. Science 1976; 193, 673-675.
11) Noedl H, Attlmayr B, Wernsdorfer W H, Kollaritsch H, Miller R S. A histidine-rich protein 2-based malaria drug sensitivity assay for field use. Am. J. Trop. Med. Hyg. 2004; 71, 711-714.
12) Noedl H, Wernsdorfer, W H, Miller R S, Wongsrichanalai C. Histidine-rich protein II: a novel approach to malaria drug sensitivity testing. Antimicrob. Agents Chemother. 2002; 46, 1658-1664.
13) Noedl H, Wongsrichanalai C, Miller R S, Myint K S, Looareesuwan S, Sukthana Y, Wongchotigul V, Kollaritsch H, Wiedermann G, Wernsdorfer W H. Plasmodium falciparum: effect of anti-malarial drugs on the production and secretion characteristics of histidine-rich protein II. Exp. Parasitol. 2002; 102, 157-163.
14) Fidock A, Rosenthal P J, Croft S L, Brun R, Nwaka S. Antimalarial drug discovery: efficacy models for compound screening. Nature Reviews Drug Discovery (2004), 3(6), 509-520. Supplemental file http://www.mmv.org/FilesUpld/164.pdf.
15) Tornoe C W, Christensen C, Meldal M. Peptidotriazoles on solid phase: [1,2,3]-triazoles by regiospecific copper(I)-catalyzed 1,3-dipolar cycloadditions of terminal alkynes to azides. J. Org. Chem. 2002; 67(9), 3057-64.

1. A compound for use in the treatment of disorders mediated by protozoan organisms, the compound being of formula I 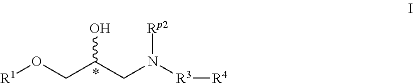
wherein,
R¹ is a group of formula II 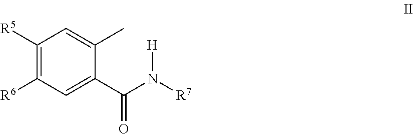
R² is H, (C₁₋₈) alkyl, or (C₃₋₆) cycloalkyl, wherein alkyl or cycloalkyl optionally are substituted by
(C₁₋₄) alkyl,
(C₁₋₄) alkoxy, or
phenyl, wherein phenyl optionally is substituted by one or more (C₁₋₆) alkoxy groups,
R³ is not present, or R³ is (C₁₋₈) alkylene, wherein alkylene is unsubstituted or substituted by (C₁₋₈) alkyl; or
R² and R³ together with the nitrogen atom to which they are attached form a heterocyclic ring, optionally comprising a further heteroatom,
R⁴ is
H, if R³ is present,
(C₅₋₁₂) cycloalkyl, wherein cycloalkyl optionally is substituted by (C₁₋₄) alkyl or hydroxy;
(C₁₋₄) alkyl, optionally substituted by phenyl,
if R³ is present, R⁴ is (C₆₋₁₂) aryl, wherein aryl optionally is substituted by (C₁₋₆) alkyl, (C₂₋₆) alkenyl, (C₂₋₆) alkynyl, (C₁₋₆) alkyloxy, (C₂₋₆) alkenyl-(C₁₋₄) alkylenoxy, HC≡C—(C₁₋₆) alkylenoxy, halogen, halogenated (C₁₋₄) alkyl, phenylcarbonyl, or 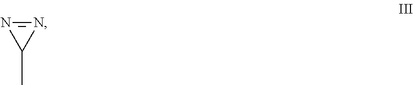
diazirinyl of formula III
R⁵ and R⁶ independently of each other are H, halogen, (C₁₋₄) alkyl, (C₂₋₄) alkenyl, (C₂₋₄) alkynyl, (C₁₋₄) alkoxy, (C₂₋₆) alkenyl-(C₁₋₄) alkylenoxy, or HC≡C—(C₁₋₆) alkylenoxy,
or
R⁵ and R⁶ together with the phenyl to which they are attached form an aromatic ring system; and
R⁷ is
(C₁₋₈) alkyl or (C₆₋₁₂) aryl, wherein alkyl is unsubstituted or substituted and aryl is substituted by
halogen,
(C₁₋₄) alkyl, (C₂₋₄) alkenyl, (C₂₋₄) alkynyl,
halogenated (C₁₋₄) alkyl,
(C₁₋₄) alkoxy, (C₂₋₆) alkenyl-(C₁₋₄) alkylenoxy, HC≡C—(C₁₋₆) alkylenoxy, or
(C₆₋₁₂) aryl, wherein aryl is substituted by halogen, (C₁₋₄) alkyl, (C₂₋₄) alkenyl, (C₂₋₄) alkynyl, halogenated (C₁₋₄) alkyl, e.g. CF₃, (C₁₋₄) alkoxy, (C₂₋₆) alkenyl-(C₁₋₄) alkylenoxy, HC≡C—(C₁₋₆)alkylenoxy, phenylcarbonyl, or diazirinyl of formula III

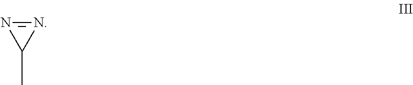

2. The compound of claim 1, wherein in the compound of formula I:
R¹ is as defined in claim 1,
R² is H or (C₁₋₈) alkyl, wherein alkyl optionally is substituted by (C₁₋₄) alkoxyphenyl or (C₃₋₆) cycloalkyl,
R³ is not present, or R³ is (C₁₋₈) alkylene, or
R² and R³ together with the nitrogen atom to which they are attached form piperidinyl or piperazinyl;
R⁴ is
hydrogen,
(C₈₋₁₂) cycloalkyl, wherein cycloalkyl optionally is substituted by (C₁₋₄) alkyl,
(C₁₋₄) alkyl, substituted by phenyl,
unsubstituted phenyl, or phenyl substituted by (C₁₋₆)alkyl, phenylcarbonyl, diazirinyl of formula III, halogenated (C₁₋₄) alkyl, (C₁₋₄) alkoxy or HC≡C—(C₁₋₆) alkylenoxy,
R⁵ and R⁶ independently of each other are H, halogen or HC≡C—(C₁₋₆) alkylenoxy, and
R⁷ is
(C₁₋₈)alkyl, wherein alkyl optionally is substituted by (C₁₋₄) alkoxyphenyl, halogenated phenyl, phenyl substituted by halogenated (C₁₋₄) alkyl, diazirinyl of formula III or (C₁₋₆) alkylphenyl, or
(C₆₋₁₂) aryl, including naphthalinyl and phenyl, wherein phenyl is substituted by halogen, (C₁₋₆) alkyl, or halogenated (C₁₋₄) alkyl.

3. The compound of claim 1, wherein in the compound of formula I:
R⁴ is (C₈₋₁₂) cycloalkyl and
R⁷ is (C₁₋₈) alkyl or (C₆₋₁₂) aryl,
wherein R⁷ as alkyl is unsubstituted or substituted by (C₆₋₁₂) aryl, and
wherein R⁷ as aryl is substituted by
halogen,
(C₁₋₄) alkyl, (C₂₋₄) alkenyl, (C₂₋₄) alkynyl,
halogenated (C₁₋₄) alkyl,
(C₁₋₄) alkoxy, (C₂₋₆) alkenyl-(C₁₋₄) alkylenoxy, HC≡C—(C₁₋₆) alkylenoxy, or
(C₆₋₁₂) aryl, which aryl is substituted by halogen, (C₁₋₄) alkyl, (C₂₋₄) alkenyl, (C₂₋₄) alkynyl, halogenated (C₁₋₄) alkyl, e.g. CF₃, (C₁₋₄) alkoxy, (C₂₋₆) alkenyl-(C₁₋₄) alkylenoxy, HC≡C—(C₁₋₆)alkylenoxy, phenylcarbonyl, or diazirinyl of formula

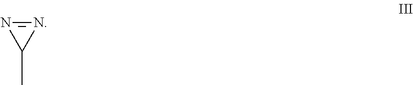

4. The compound of claim 1, wherein the compound is of formula Ip: 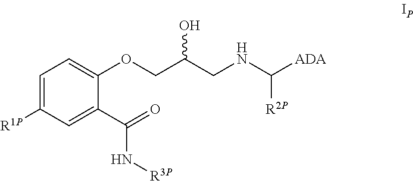
wherein,
ADA is adamantyl, which adamantyl optionally is substituted by (C₁₋₄) alkyl, or hydroxy,
R^{1P} is hydrogen or halogen,
R^{2P} is
hydrogen
(C₁₋₈) alkyl, including unsubstituted alkyl, or alkyl substituted by phenyl, or
(C₃₋₆) cycloalkyl, and
R^{3P} is
(C₆₋₁₂) aryl, which aryl is unsubstituted or substituted, including aryl substituted by one or more of groups
halogen,
(C₁₋₄) alkyl,
(C₂₋₄) alkenyl,
halo(C₁₋₄) alkyl,
(C₁₋₄) alkoxy, or
(C₁₋₁₂) alkyl, which alkyl is unsubstituted or substituted by (C₆₋₁₂) aryl, including aryl substituted by a group of formula 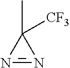
or aryl substituted as in the meaning of R^{3P}.

5. The compound of claim 1, wherein the compound is selected from:
N-Benzyl-2-(3-cyclododecylamino-2-hydroxy-propoxy)-benzamide of formula I-52 or N-Benzyl-2-(3-cyclooctylamino-2-hydroxy-propoxy)-benzamide of formula I-53.

6. The compound of claim 1, wherein the compound is a compound of formula VIII: 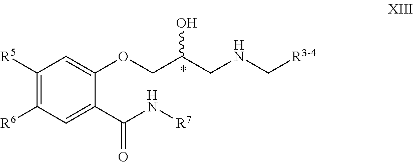
wherein
R³⁻⁴ is 4-benzoylphenyl or a diazirinylphenyl of formula 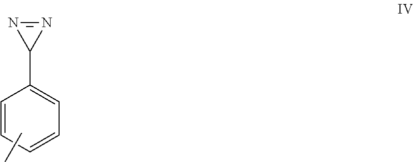
and R⁵, R⁶ and R⁷ are as defined in claim 1.

7. The compound of claim 1, wherein the compound is a compound of formula XIV: 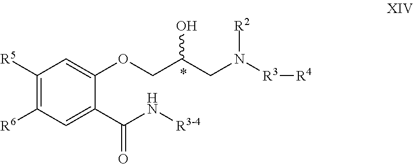
wherein
R³⁻⁴ is 4-benzoylphenyl or methyl, substituted by a diazirinylphenyl of formula IV: 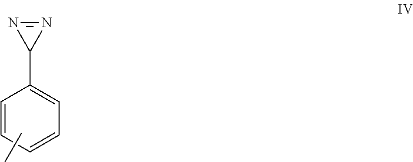
and R², R³, R⁴, R⁵ and R⁶ are as defined in claim 1.

8. The compound of claim 1, which is in the form of a pharmaceutically acceptable salt.

9. A method of using the compound of claim 1, comprising administering a pharmaceutically acceptable quantity of the compound to a patient in need thereof.

10. A pharmaceutical composition comprising the compound of claim 1 in association with at least one pharmaceutical excipient.

11. A method of treating disorders mediated by protozoan organisms, which treatment comprises administering to a subject in need of such treatment an effective amount of the compound of claim 1.

12. The compound of claim 1, wherein the compound comprises a photoaffinity label configured for the identification of the molecular target(s) of arylamino alcohol containing drugs.

13. A compound for use in the treatment of disorders mediated by protozoan organisms, wherein the compound is selected from the group consisting of:
5-Chloro-2-hydroxy-N-naphthalen-1-ylmethyl-benzamide,
N-(4-Fluoro-phenyl)-2-hydroxy-5-prop-2-ynyloxy-benzamide,
N-(2-Methoxy-benzyl)-2-oxiranylmethoxy-benzamide,
N-(4-Methoxy-benzyl)-2-oxiranylmethoxy-benzamide,
2-Oxiranylmethoxy-N-phenethyl-benzamide,
5-Chloro-2-oxiranylmethoxy-N-phenethyl-benzamide,
2-(Oxiran-2-ylmethoxy)-N-(3-phenylpropyl)benzamide,
N-(3-Methyl-butyl)-2-oxiranylmethoxy-benzamide,
2-Oxiranylmethoxy-N-(4-trifluoromethyl-benzyl)-benzamide,
5-Chloro-N-naphthalen-1-ylmethyl-2-oxiranylmethoxy-benzamide,
N-(4-Fluoro-phenyl)-2-oxiranylmethoxy-benzamide,
N-(2-Fluoro-phenyl)-2-oxiranylmethoxy-benzamide,
N-(2,4-Difluoro-phenyl)-2-oxiranylmethoxy-benzamide,
2-Oxiranylmethoxy-N-(3-trifluoromethyl-phenyl)-benzamide,
N-(4-Fluoro-phenyl)-2-oxiranylmethoxy-5-prop-2-ynyloxy-benzamide,
N-(2-Allyl-phenyl)-2-oxiranylmethoxy-benzamide,
5-Chloro-N-(4-fluoro-phenyl)-2-oxiranylmethoxy-benzamide,
5-Chloro-2-oxiranylmethoxy-N-(3-trifluoromethyl-phenyl)-benzamide,
N-Naphthalen-2-yl-2-oxiranylmethoxy-benzamide,
N-(2-Allyl-phenyl)-2-hydroxy-benzamide,
2-[3-(Adamantan-2-yl-methyl-amino)-2-hydroxy-propoxy]-benzoic acid methyl ester (INT-14), and
2-[3-(Adamantan-2-yl-methyl-amino)-2-hydroxy-propoxy]-benzoic acid (INT-15);
particularly
2-[3-(Adamantan-2-yl-methyl-amino)-2-hydroxy-propoxy]-benzoic acid methyl ester (INT-14), and
2-[3-(Adamantan-2-yl-methyl-amino)-2-hydroxy-propoxy]-benzoic acid (INT-15).

14. A compound of formula XIII: 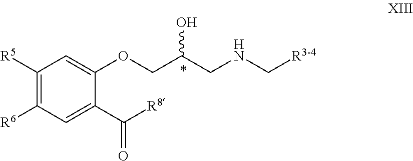
wherein,
R³⁻⁴ is a diazirinylphenyl of formula IV as defined in claim 6 or benzoylphenyl, and
R8′ is methoxy or ethoxy.

15. A compound of formula I_{PRIO}: 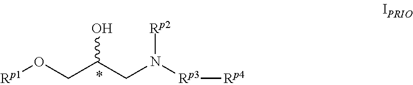
wherein,
R^{p1} is a group of formula II_{PRIO}: 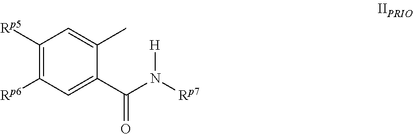
wherein,
R^{p2} is H, (C₁₋₈) alkyl, or (C₃₋₆) cycloalkyl, wherein alkyl or cycloalkyl optionally are substituted by
(C₁₋₄) alkyl,
(C₁₋₄) alkoxy, or
phenyl, wherein phenyl optionally is substituted by one or more (C₁₋₆) alkoxy groups,
R^{p3} is not present or is (C₁₋₈) alkylene, wherein alkylene is unsubstituted or substituted by (C₁₋₈) alkyl; or
R^{p2} and R^{p3} together with the nitrogen atom to which they are attached form a heterocyclic ring, optionally comprising a further heteroatom,
R^{p4} is
(C₅₋₁₂) cycloalkyl, wherein cycloalkyl optionally is substituted by (C₁₋₄) alkyl;
R^{p5} and R^{p6} independently of each other are H, halogen, (C₁₋₄) alkyl, (C₂₋₄) alkenyl, (C₂₋₄) alkynyl, (C₁₋₄) alkoxy, (C₂₋₆) alkenyl-(C₁₋₄) alkylenoxy, or HC≡C—(C₁₋₆) alkylenoxy, and
R^{p7} is
(C₁₋₈) alkyl or (C₆₋₁₂) aryl, wherein alkyl or aryl is unsubstituted or substituted by
halogen,
(C₁₋₄) alkyl, (C₂₋₄) alkenyl, (C₂₋₄) alkynyl,
halogenated (C₁₋₄) alkyl,
(C₁₋₄) alkoxy, (C₂₋₆) alkenyl-(C₁₋₄) alkylenoxy, HC≡C—(C₁₋₆) alkylenoxy, or
(C₆₋₁₂) aryl, wherein aryl optionally is substituted by halogen, (C₁₋₄) alkyl, (C₂₋₄) alkenyl, (C₂₋₄) alkynyl, halogenated (C₁₋₄) alkyl, (C₁₋₄) alkoxy, (C₂₋₆) alkenyl-(C₁₋₄) alkylenoxy, HC≡C—(C₁₋₆) alkylenoxy, phenylcarbonyl, or diazirinyl of formula III.

16. The compound of claim 1, wherein the compound is selected from the group consisting of:
2-[3-(Adamantan-1-ylamino)-2-hydroxy-propoxy]-N-benzyl-benzamide of formula I-1,
2-[3-(Adamantan-1-ylamino)-2-hydroxy-propoxy]-N-(2-methoxy-benzyl)-benzamide of formula I-2,
2-[3-(Adamantan-1-ylamino)-2-hydroxy-propoxy]-N-(4-fluoro-phenyl)-benzamide of formula I-3,
2-[3-(3,5-Dimethyl-adamantan-1-ylamino)-2-hydroxy-propoxy]-N-(4-fluoro-phenyl)-benzamide of formula I-4,
2-[3-(4-Adamantan-1-yl-piperazin-1-yl)-2-hydroxy-propoxy]-N-(4-fluoro-phenyl)-benzamide of formula I-5,
2-[3-(Adamantan-2-ylamino)-2-hydroxy-propoxy]-N-benzyl-benzamide of formula I-6,
2-[3-(Adamantan-2-ylamino)-2-hydroxy-propoxy]-N-(2-methoxy-benzyl)-benzamide of formula I-7,
2-[3-(Adamantan-2-ylamino)-2-hydroxy-propoxy]-N-(4-methoxy-benzyl)-benzamide of formula I-8,
2-[3-(Adamantan-2-ylamino)-2-hydroxy-propoxy]-N-phenethyl-benzamide of formula I-9,
2-[3-(Adamantan-2-ylamino)-2-hydroxy-propoxy]-5-chloro-N-phenethyl-benzamide of formula I-10,
2-[3-(Adamantan-2-ylamino)-2-hydroxy-propoxy]-N-(3-phenyl-propyl)-benzamide of formula I-11,
2-[3-(Adamantan-2-ylamino)-2-hydroxy-propoxy]-N-propyl-benzamide of formula I-12,
2-[3-(Adamantan-2-yl-methyl-amino)-2-hydroxy-propoxy]-N-benzyl-benzamide of formula I-13,
2-[3-(Adamantan-2-yl-methyl-amino)-2-hydroxy-propoxy]-N-(4-trifluoromethyl-benzyl)-benzamide of formula I-16,
2-[3-(Adamantan-2-yl-propyl-amino)-2-hydroxy-propoxy]-N-benzyl-benzamide of formula I-17,
2-[3-(Adamantan-2-yl-propyl-amino)-2-hydroxy-propoxy]-N-(2-methoxy-benzyl)-benzamide of formula I-18,
2-[3-(Adamantan-2-yl-pentyl-amino)-2-hydroxy-propoxy]-N-benzyl-benzamide of formula I-19,
2-[3-(Adamantan-2-yl-cyclopropyl-amino)-2-hydroxy-propoxy]-N-benzyl-benzamide of formula I-20,
2-{3-[Adamantan-2-yl-(2-methoxy-benzyl)-amino]-2-hydroxy-propoxy}-N-benzyl-benzamide of formula I-21,
2-[3-(Adamantan-2-yl-phenethyl-amino)-2-hydroxy-propoxy]-N-benzyl-benzamide of formula I-22,
2-{3-[Adamantan-2-yl-(3-phenyl-propyl)-amino]-2-hydroxy-propoxy}-N-benzyl-benzamide of formula I-23,
2-{3-[(Adamantan-1-ylmethyl)-amino]-2-hydroxy-propoxy}-N-(3-methyl-butyl)-benzamide of formula I-24,
2-{3-(1-Adamantan-1-yl-ethylamino)-2-hydroxy-propoxy}-N-(3-methyl-butyl)-benzamide of formula I-25,
2-{3-[(Adamantan-1-ylmethyl)-amino]-2-hydroxy-propoxy}-N-(3-methyl-butyl)-5-prop-2-ynyloxy-benzamide of formula I-26,
2-{3-[(Adamantan-1-ylmethyl)-amino]-2-hydroxy-propoxy}-N-benzyl-benzamide of formula I-27,
2-[3-(1-Adamantan-1-yl-ethylamino)-2-hydroxy-propoxy]-N-benzyl-benzamide of formula I-28,
2-{3-[(Adamantan-1-ylmethyl)-amino]-2-hydroxy-propoxy}-N-(2-methoxy-benzyl)-benzamide of formula I-29,
2-{3-[(Adamantan-1-ylmethyl)-amino]-2-hydroxy-propoxy}-N-(4-trifluoromethyl-benzyl)-benzamide of formula I-30,
2-{3-[(Adamantan-1-ylmethyl)-amino]-2-hydroxy-propoxy}-N-phenethyl-benzamide of formula I-31,
2-{3-[(Adamantan-1-ylmethyl)-amino]-2-hydroxy-propoxy}-N-(3-phenyl-propyl)-benzamide of formula I-32,
2-{3-[(Adamantan-1-ylmethyl)-amino]-2-hydroxy-propoxy}-5-chloro-N-phenethyl-benzamide of formula I-33,
2-{3-[(Adamantan-1-ylmethyl)-amino]-2-hydroxy-propoxy}-5-chloro-N-naphthalen-1-ylmethyl-benzamide of formula I-34,
2-[3-(1-Adamantan-1-yl-ethylamino)-2-hydroxy-propoxy]-5-chloro-N-naphthalen-1-ylmethyl-benzamide of formula I-35,
2-[3-(Adamantan-1-ylmethyl-methyl-amino)-2-hydroxy-propoxy]-N-benzyl-benzamide of formula I-36,
2-[3-(Adamantan-1-ylmethyl-methyl-amino)-2-hydroxy-propoxy]-N-(4-fluoro-phenyl)-benzamide of formula I-37,
2-{3-[(Adamantan-1-ylmethyl)-amino]-2-hydroxy-propoxy}-N-(2-fluoro-phenyl)-benzamide of formula I-38,
2-{3-[(Adamantan-1-ylmethyl)-amino]-2-hydroxy-propoxy}-N-(2,4-difluoro-phenyl)-benzamide of formula I-39,
2-{3-[(Adamantan-1-ylmethyl)-amino]-2-hydroxy-propoxy}-N-p-tolyl-benzamide of formula I-40,
2-[3-(1-Adamantan-1-yl-ethylamino)-2-hydroxy-propoxy]-N-p-tolyl-benzamide of formula I-41,
2-{3-[(Adamantan-1-ylmethyl)-amino]-2-hydroxy-propoxy}-N-(3-trifluoromethyl-phenyl)-benzamide of formula I-42,
2-[3-(Adamantan-2-yl-methyl-amino)-2-hydroxy-propoxy]-N-[4-(3-trifluoromethyl-3H-diazirin-3-yl)-benzyl]-benzamide of formula I-43,
2-{2-Hydroxy-3-[4-(3-trifluoromethyl-3H-diazirin-3-yl)-benzylamino]-propoxy}-N-(3-methyl-butyl)-5-prop-2-ynyloxy-benzamide of formula I-44,
N-(4-Fluoro-phenyl)-2-{2-hydroxy-3-[4-(3-trifluoromethyl-3H-diazirin-3-yl)-benzylamino]-propoxy}-5-prop-2-ynyloxy-benzamide of formula I-45,
N-(4-Fluoro-phenyl)-2-[2-hydroxy-3-(4-phenyl-piperidin-1-yl)-propoxy]-benzamide of formula I-46,
2-[3-(4-Benzhydryl-piperazin-1-yl)-2-hydroxy-propoxy]-N-(4-fluoro-phenyl)-benzamide of formula I-48,
2-{3-[4-(2,3-Dimethyl-phenyl)-piperazin-1-yl]-2-hydroxy-propoxy}-N-(4-methoxy-benzyl)-benzamide of formula I-51,
2-{3-[(Adamantan-1-ylmethyl)-amino]-2-hydroxy-propoxy}-N-(4-fluoro-phenyl)-benzamide of formula I-55,
2-[3-(2-Adamantan-1-yl-ethylamino)-2-hydroxy-propoxy]-N-benzyl-benzamide of formula I-62,
2-[3-(Adamantan-2-yl-methyl-amino)-2-hydroxy-propoxy]-N-(4-tert-butyl-benzyl)-benzamide of formula I-64,
2-{3-[(Adamantan-1-ylmethyl)-amino]-2-hydroxy-pro-poxyl}-5-chloro-N-(4-fluoro-phenyl)-benzamide of formula I-66,
2-[2-Hydroxy-3-(4-phenyl-piperidin-1-yl)-propoxy]-N-(3-trifluoromethyl-phenyl)-benzamide of formula I-67,
2-[3-(4-Benzyl-piperidin-1-yl)-2-hydroxy-propoxy]-N-(3-trifluoromethyl-phenyl)-benzamide of formula I-68,
2-[3-(4-Benzhydryl-piperazin-1-yl)-2-hydroxy-prop-oxy]-N-(3-trifluoromethyl-phenyl)-benzamide of formula I-69,
2-[2-Hydroxy-3-(3-hydroxy-adamantan-1-ylamino)-propoxy]-N-(3-trifluoromethyl-phenyl)-benzamide of formula I-70,
2-{3-[(Adamantan-1-ylmethyl)-amino]-2-hydroxy-propoxy}-N-naphthalen-2-yl-benzamide of formula I-71,
2-{3-[(Adamantan-1-ylmethyl)-amino]-2-hydroxy-propoxy}-5-chloro-N-(3-trifluoromethyl-phenyl)-benzamide of formula I-72,
2-{3-[(Adamantan-1-ylmethyl)-amino]-2-hydroxy-propoxy}-N-(2-allyl-phenyl)-benzamide of formula I-73,
2-{3-[(Adamantan-1-ylmethyl)-amino]-2-hydroxy-propoxy}-N-(4-bromo-phenyl)-benzamide of formula I-74,
2-[3-(Adamantan-1-ylamino)-2-hydroxy-propoxy]-N-(4-bromo-phenyl)-benzamide of formula I-75,
N-(4-Bromo-phenyl)-2-[2-hydroxy-3-(3-hydroxy-adamantan-1-ylamino)-propoxy]-benzamide of formula I-76,
N-(2-Fluoro-phenyl)-2-[2-hydroxy-3-(4-phenyl-piperidin-1-yl)-propoxy]-benzamide of formula I-77,
2-[3-(4-Benzyl-piperidin-1-yl)-2-hydroxy-propoxy]-N-(2-fluoro-phenyl)-benzamide of formula I-78,
2-[3-(4-Benzhydryl-piperazin-1-yl)-2-hydroxy-propoxy]-N-(2-fluoro-phenyl)-benzamide of formula I-79,
2-[2-Hydroxy-3-(4-phenyl-piperidin-1-yl)-propoxy]-N-p-tolyl-benzamide of formula I-80,
2-[3-(4-Benzyl-piperidin-1-yl)-2-hydroxy-propoxy]-N-p-tolyl-benzamide of formula I-81,
2-[3-(4-Benzhydryl-piperazin-1-yl)-2-hydroxy-propoxy]-N-p-tolyl-benzamide of formula I-82,
5-Chloro-2-{2-hydroxy-3-(4-phenyl-piperidin-1-yl)-propoxy}-N-(3-trifluoromethyl-phenyl)-benzamide of formula I-83,
2-[3-(4-Benzyl-piperidin-1-yl)-2-hydroxy-propoxy]-5-chloro-N-(3-trifluoromethyl-phenyl)-benzamide of formula I-84,
2-[3-(4-Benzhydryl-piperazin-1-yl)-2-hydroxy-propoxy]-5-chloro-N-(3-trifluoromethyl-phenyl)-benzamide of formula I-85,
3-{3-[(Adamantan-1-ylmethyl)-amino]-2-hydroxy-propoxy}-naphthalene-2-carboxylic acid (4-fluoro-phenyl)-amide of formula I-86,
3-[3-(Adamantan-1-ylamino)-2-hydroxy-propoxy]-naphthalene-2-carboxylic acid (4-fluoro-phenyl)-amide of formula I-87,
5-Bromo-N-(4-fluoro-phenyl)-2-{2-hydroxy-3-(4-phenyl-piperidin-1-yl)-propoxy}-benzamide of formula I-88,
2-[3-(4-Benzyl-piperidin-1-yl)-2-hydroxy-propoxy]-5-bromo-N-(4-fluoro-phenyl)-benzamide of formula I-89,
2-[3-(4-Benzhydryl-piperazin-1-yl)-2-hydroxy-propoxy]-5-bromo-N-(4-fluoro-phenyl)-benzamide of formula I-90, 2-{3-[(Adamantan-1-ylmethyl)-amino]-2-hydroxy-propoxy}-5-bromo-N-(4-fluoro-phenyl)-benzamide of formula I-91, and
2-[3-(Adamantan-1-ylamino)-2-hydroxy-propoxy]-5-bromo-N-(4-fluoro-phenyl)-benzamide of formula I-92.

17. The compound of claim 16, which is in the form of a pharmaceutically acceptable salt.

18. A compound for use in the treatment of disorders mediated by protozoan organisms, the compound being of formula I: 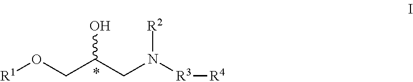
wherein,
R¹ is a group of formula II: 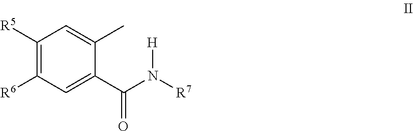
R² is H, (C₁₋₈) alkyl, or (C₃₋₆) cycloalkyl, wherein alkyl or cycloalkyl optionally are substituted by
(C₁₋₄) alkyl,
(C₁₋₄) alkoxy, or
phenyl, wherein phenyl optionally is substituted by one or more (C₁₋₆) alkoxy groups,
R³ is not present, or R³ is (C₁₋₈) alkylene, wherein alkylene is unsubstituted or substituted by (C₁₋₈) alkyl; or
R² and R³ together with the nitrogen atom to which they are attached form a heterocyclic ring, optionally comprising a further heteroatom,
R⁴ is
(C₈₋₁₂) cycloalkyl, wherein cycloalkyl optionally is substituted by (C₁₋₄) alkyl or hydroxy;
R⁵ and R⁶ independently of each other are H, halogen, (C₁₋₄) alkyl, (C₂₋₄) alkenyl, (C₂₋₄) alkynyl, (C₁₋₄) alkoxy, (C₂₋₆) alkenyl-(C₁₋₄) alkylenoxy, or HC≡C—(C₁₋₆) alkylenoxy,
or
R⁵ and R⁶ together with the phenyl to which they are attached form an aromatic ring system; and
R⁷ is
(C₁₋₈) alkyl or (C₆₋₁₂) aryl, wherein alkyl is unsubstituted or substituted and aryl is substituted by
halogen,
(C₁₋₄) alkyl, (C₂₋₄) alkenyl, (C₂₋₄) alkynyl,
halogenated (C₁₋₄) alkyl,
(C₁₋₄) alkoxy, (C₂₋₆) alkenyl-(C₁₋₄) alkylenoxy, HC≡C—(C₁₋₆) alkylenoxy, or
(C₆₋₁₂) aryl, wherein aryl is substituted by halogen, (C₁₋₄) alkyl, (C₂₋₄) alkenyl, (C₂₋₄) alkynyl, halogenated (C₁₋₄) alkyl, e.g. CF₃, (C₁₋₄) alkoxy, (C₂₋₆) alkenyl-(C₁₋₄) alkylenoxy, HC≡C—(C₁₋₆)alkylenoxy, phenylcarbonyl, or diazirinyl of formula III [chemical structure]
